(19) United States
(12) Patent Application Publication
(10) Pub. No.: US 2023/0364013 A1
(43) Pub. Date: Nov. 16, 2023

(54) STABLE ORAL DISPERSIBLE FORMULATION FOR EPINEPHRINE

(71) Applicant: Catalent U.K. Swindon Zydis Limited, Glasgow (GB)

(72) Inventors: Leon Paul GROTHER, Swindon (GB); Nicholas Walker BARRAS, Swindon (GB); Philip AXE, Magor, Caldicot (GB); Michael John HUTCHINSON, Lisburn (GB)

(73) Assignee: Catalent U.K. Swindon Zydis Limited, Glasgow (GB)

(21) Appl. No.: 18/030,541

(22) PCT Filed: Oct. 7, 2021
(86) PCT No.: PCT/EP2021/077711

§ 371 Date: Apr. 6, 2023

Related U.S. Application Data
(60) Provisional application No. 63/089,309, filed on Oct. 8, 2020.

(51) Int. Cl. A61K 9/00; A61K 31/137; A61K 9/20

(52) U.S. Cl. CPC A61K 9/0056; A61K 31/137; A61K 9/2063; A61K 9/2009; A61K 9/2018; A61K 9/2095

(57) ABSTRACT

The present disclosure is directed to oral dosage forms and processes for producing the oral dosage forms. The dosage forms include an Epinephrine hormone or pharmaceutically acceptable salt or solvate thereof, an antioxidant, and a chelating agent. The antioxidant can be sodium metabisulfate and the chelating agent can be edetate disodium (“EDTA”).

### CROSS-REFERENCE TO RELATED APPLICATIONS

This application is a national stage application under 35 U.S.C. 371 of International Application No. PCT/EP2021/077711, filed Oct. 7, 2021, which claims priority to and the benefit of U.S. Provisional Application No. 63/089,309, filed on Oct. 8, 2020, the entire contents of each priority application is incorporated herein by reference in their entireties.

### FIELD

This disclosure relates to a formulation for oral delivery of Epinephrine hormone and derivatives or salts or solvates thereof. More specifically, this relates to a chemically stable pharmaceutical formulation intended for oral delivery of an Epinephrine hormone and derivatives or pharmaceutically acceptable salt or solvate thereof in a freeze-dried dosage form.

### BACKGROUND

Epinephrine is a non-selective α- and β-adrenergic receptor agonist used for emergency treatment of anaphylaxis. Epinephrine (i.e., adrenaline) is known to be sensitive to light, oxygen, humidity, and temperature. When epinephrine is in its raw form of a solid crystalline state and in a sealed container, it is considered stable under standard temperature and pressure conditions. The most common use of epinephrine is in an EpiPen. EpiPen is an injection containing epinephrine that is mostly used to narrow blood vessels and open airways in lungs for people suffering from severe allergies.

An EpiPen injector contains the active ingredient adrenaline, which is a hormone produced naturally by the body to deal with life-threatening situations. An injection of adrenaline can control the symptoms of active allergic reactions. Unfortunately, this delivery device is an injection so it must break the skin of a person's body in order for the active pharmaceutical ingredient (“API”) to reach the blood stream. Such parenteral administration may not be ideal for certain situations especially those in which the patient or person to be treated has a phobia for needles or injections.

### SUMMARY

Applicants have discovered formulations for orally administering Epinephrine or salts or solvates thereof. Specifically, Applicants discovered a freeze-dried, suspension-based epinephrine formulation that has suitable stability such that it could compete with other established pharmaceutical formulations/products for the delivery of Epinephrine (such as the EpiPen product). As with the EpiPen product, sodium metabisulfite (i.e., antioxidant) was included in the formulation at a range of concentrations over various formulation development studies. In order to solubilize the API, the pH was adjusted to less than 6.

After numerous attempts to create solution-based formulations, a freeze-dried, suspension-based formulation was developed and was considered preferable with regards to physical and chemical stability, thus ensuring theoretical safety and efficacy on administration to humans. It was believed that the freeze-dried finished product would have sufficient stability to ensure suitable shelf life without the requirement for refrigerated storage. To compare, the EpiPen product has a shelf life of 18 months.

Analytical studies conducted on a freeze-dried, solution-based formulations, as well as observations during manufacture indicated a lack of adequate stability both in-process (during manufacture, solution holding period) and in the finished product. The levels of sodium metabisulfite and formulation pH levels were ranged over numerous studies; however, all attempts to achieve stability in freeze-dried, solution-based Epinephrine product failed. Applicants suspected this to be a result of the instability of the API when freeze-dried in solution form. This applied both to the manufacturing process in which the drug was in solution, and in the finished dosage form in which it is known that moisture remains at low levels. The inherent porous nature of the dosage forms prepared by the freeze-drying process disclosed herein is also suspected to be a contributing factor.

Other tests were performed on the EpiPen product in order to continue to develop a freeze-dried, solution-based epinephrine product. For example, it was noted that the EpiPen product was able to be degassed, sealed in air-tight packaging, and formulated at a low pH (˜3.5). Such conditions and processes provided enhance stability to the EpiPen product, but were not feasible for the dosage forms prepared by the freeze-drying process disclosed herein.

Following the inability to achieve suitable stability for a freeze-dried, solution-based epinephrine product, the formulation strategy was altered to target a freeze-dried, suspension-based epinephrine product. As used herein, a “solution-based” formulation is a formulation in which the majority of the epinephrine is dissolved within the formulation following mixing and prior to freezing and subsequent processing steps. As used herein, a “suspension-based” formulation is a formulation in which the majority of the epinephrine is present within the formulation as undissolved, solid particles following mixing and prior to freezing and subsequent processing. This strategy was selected because the epinephrine API is known to be stable when in its solid (crystalline) state.

In some embodiments, an oral solid dosage form includes an Epinephrine hormone or pharmaceutically acceptable salt or solvate thereof; 10-40 wt. % of a matrix former; 10-40 wt. % of a structure former; an antioxidant; and a pH modifier. In some embodiments, the antioxidant comprises sodium metabisulfite. In some embodiments, the dosage form comprises 0.1-5 wt. % of the antioxidant. In some embodiments, the dosage form further includes a chelating agent. In some embodiments, the chelating agent comprises edetate disodium (“EDTA”). In some embodiments, the dosage form comprises 0.1-1 wt. % the chelating agent. In some embodiments, the matrix former comprises gelatin, pullulan, starch, or combinations thereof. In some embodiments, the gelatin comprises fish gelatin, bovine gelatin, porcine gelatin, or combination thereof. In some embodiments, the gelatin is fish gelatin and the fish gelatin is high molecular weight fish gelatin. In some embodiments, the structure former comprises mannitol. In some embodiments, the Epinephrine hormone or pharmaceutically acceptable salt or solvate thereof comprises Epinephrine maleate, Epinephrine tartrate, Epinephrine Bitartrate, or Epinephrine hydrochloride. In some embodiments, the dosage form comprises a pharmaceutically effective amount of the Epinephrine hormone or pharmaceutically acceptable salt or solvate thereof. In some embodiments, the dosage form comprises 15-70 wt. % of the Epinephrine hormone or pharmaceutically acceptable salt or solvate thereof. In some embodiments, the pH modifier is sodium hydroxide. In some embodiments, the dosage form comprises 0.1-2 wt. % pH modifier. In some embodiments, the dosage form includes a sweetener. In some embodiments, the sweetener is sucralose. In some embodiments, the dosage form comprises 0.1-5 wt. % sweetener.

In some embodiments, a method of treating a patient includes placing any of the dosage forms described in the previous paragraph in an oral cavity of a person in need of the treatment. In some embodiments, the placement in the oral cavity is placement on or under the tongue or in the buccal or pharyngeal region.

In some embodiments, a method of forming an oral solid dosage form includes dosing a pharmaceutical formulation into a preformed mold, wherein the pharmaceutical formulation has a pH of 7.5-9.5 and the pharmaceutical formulation comprises: an Epinephrine hormone or pharmaceutically acceptable salt or solvate thereof; 1-10 wt. % matrix former; 1-10 wt. % of a structure former; 0.1-1 wt. % antioxidant; and a pH modifier;

freezing the dosed pharmaceutical formulation; and freeze-drying the annealed pharmaceutical formulation to form the dosage form. In some embodiments, the dosed pharmaceutical formulation is frozen at a temperature of −40° C. to −120° C. for a duration of about 1-5 minutes. In some embodiments, the pharmaceutical formulation comprises a 0.01-0.1 wt. % chelating agent. In some embodiments, the pH modifier is sodium hydroxide. In some embodiments, the pharmaceutical formulation has a pH of 8.4-8.6. In some embodiments, the matrix former comprises gelatin. In some embodiments, the gelatin comprises fish gelatin. In some embodiments, the fish gelatin is high molecular weight fish gelatin. In some embodiments, the structure former comprises mannitol. In some embodiments, the antioxidant is sodium metabisulfite. In some embodiments, the chelating agent is EDTA. In some embodiments, the Epinephrine hormone or pharmaceutically acceptable salt or solvate thereof comprises Epinephrine maleate, Epinephrine tartrate, Epinephrine Bitartrate, or Epinephrine hydrochloride. In some embodiments, the pharmaceutical formulation comprises a sweetener. In some embodiments, the sweetener is sucralose. In some embodiments, the pharmaceutical formulation comprises a pharmaceutically effective amount of the Epinephrine hormone or pharmaceutically acceptable salt or solvate thereof. In some embodiments, the pharmaceutical formulation comprises 1-30 wt. % of the Epinephrine hormone or pharmaceutically acceptable salt or solvate thereof.

In some embodiments, a method of forming a pharmaceutical formulation includes mixing a solvent, a matrix former, and a structure former to form a pre-mix; adding an antioxidant to the pre-mix; adding a first pH modifier to the pre-mix such that the pH of the pre-mix is 7.15-9.5; wetting an Epinephrine hormone or pharmaceutically acceptable salt or solvate thereof with the pre-mix to form a suspension; adding a second pH modifier to the suspension to form the pharmaceutical formulation such that the pH of the pharmaceutical formulation is 7.15-9.5, wherein the amount of the Epinephrine hormone or pharmaceutically acceptable salt or solvate thereof dissolved in the pharmaceutical formulation is less than about 3.5 wt. % as a proportion of epinephrine content. In some embodiments, a chelating agent is also added to the pre-mix with the antioxidant. In some embodiments, the method further includes heating the pre-mix to 50-70° C. and cooling the pre-mix to 15-30° C. after mixing the solvent, the matrix former, and the structure former. In some embodiments, the pH of the pharmaceutical formulation is 8-9. In some embodiments, the amount of the Epinephrine hormone or pharmaceutically acceptable salt or solvate thereof dissolved in the pharmaceutical formulation is less than about 2 wt. % as a proportion of epinephrine content.

Additional advantages will be readily apparent to those skilled in the art from the following detailed description. The examples and descriptions herein are to be regarded as illustrative in nature and not restrictive.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

### BRIEF DESCRIPTION OF THE DRAWINGS

Exemplary embodiments are described with reference to the accompanying figures, in which:

FIG. 1 illustrates a flow chart for producing a dosage form containing epinephrine disclosed herein.

FIG. 2 illustrates images of the macroscopic appearance of a stable suspension at 0-hour (left) and 24-hour (right), having been left stirring for these respective durations (hereafter referred to as suspension hold time).

FIG. 3A illustrates images of stable suspension based freeze dried finished dosage form appearance dosed at 0-hour suspension hold time.

FIG. 3B illustrates images of stable suspension based freeze dried finished dosage form appearance dosed at 24-hour suspension hold time.

FIG. 4 illustrates images of the macroscopic appearance of an unstable suspension with no sodium metabisulfite or EDTA at 0-hour (left) and 24-hour (right) suspension hold.

FIG. 5A illustrates images of unstable suspension based freeze dried finished dosage form appearance dosed at 0-hour suspension hold time formulated with no sodium metabisulfite or EDTA.

FIG. 5B illustrates images of unstable suspension based freeze dried finished dosage form appearance dosed at 24-hour suspension hold time formulated with no sodium metabisulfite or EDTA.

FIG. 6 illustrates images of the macroscopic appearance of an unstable solution formulated with sodium metabisulfite 0-hour (left) and 24-hour (right) solution hold.

FIG. 7 illustrates images of unstable freeze dried, solution based finished dosage form appearance dosed at 0-hour solution hold time formulated with sodium metabisulfite and a pH of 6.

FIG. 8 is a table of showing the formulation details for the batches in Solution-Based Formulation Study 1 disclosed herein.

FIG. 9 is a flowchart for the production of Batches 1-2 of the Solution-Based Formulation Study 1 disclosed herein.

FIG. 10 is a flowchart for the production of Batches 3-5 of the Solution-Based Formulation Study 1 disclosed herein.

FIGS. 11A-B provide the preparation of the mixes for a Solution-Based Formulation Study disclosed herein.

FIG. 12 provides a table summarizing the dosing points, wet fill weights, and number of blisters dosed for a Study disclosed herein.

FIG. 13 provides a range of frozen hold times for a Study disclosed herein.

FIG. 14 provides the parameters used for sealing for a Study disclosed herein.

FIG. 15 provides the pH measurements that were taken for each batch at different stages of mix preparation for a Study disclosed herein.

FIG. 16 provides the appearance observations for a Study disclosed herein.

FIGS. 17A-C provide images of the macroscopic appearance of formulations of a Study disclosed herein.

FIG. 18 provides a reference picture for a “glassy” dosage form.

FIGS. 19A-C provides the appearance of dosage forms of a Study disclosed herein.

FIG. 20 provides the appearance of dosage forms of a Study disclosed herein.

FIGS. 21A-B provide the terms used to describe the unit/dosage form appearances.

FIG. 22 provides the dispersion results for a Study disclosed herein.

FIG. 23 provides the inspection categories and examples of each category.

FIGS. 24-26 shows the appearance of dosage forms following heat stress of Study disclosed herein.

FIGS. 27A-B provide a detailed breakdown of the formulation compositions of a Study disclosed herein.

FIGS. 28-29 provide flowcharts for creating dosage forms of a Study disclosed herein.

FIG. 30 provides the preparation of the mixes for a Study disclosed herein.

FIG. 31 provides a table summarizing the dosing points, wet fill weights, and number of blisters dosed for a Study disclosed herein.

FIG. 32 provides the frozen hold times of a Study disclosed herein.

FIG. 33 provides the pH measurements that were taken for each batch at different stages of mix preparation for a Study disclosed herein.

FIG. 34 provides the appearance observations for a Study disclosed herein.

FIGS. 35A-C provide images of the macroscopic appearance of formulations of a Study disclosed herein.

FIGS. 36A-C provide the appearance of dosage forms of a Study disclosed herein.

FIG. 37 provides the dispersion results for a Study disclosed herein.

FIG. 38 shows the appearance of dosage forms following heat stress of Study disclosed herein.

FIGS. 39A-B show the assay data and appearance of dosage forms following heat stress of a Study disclosed herein.

FIG. 40 shows the appearance of dosage forms following heat stress of Study disclosed herein.

FIGS. 41A-B show the assay data and appearance of dosage forms following heat stress of a Study disclosed herein.

FIGS. 42A-B show the assay data and appearance of dosage forms following heat stress of a Study disclosed herein.

FIG. 43 provides a detailed breakdown of the formulation compositions of a Study disclosed herein.

FIG. 44 provides a flowchart for creating dosage forms of a Study disclosed herein.

FIG. 45 provides the preparation of the mixes for a Study disclosed herein.

FIG. 46 provides the pH measurements that were taken for each batch at different stages of mix preparation for a Study disclosed herein.

FIGS. 47A-B provide the appearance observations during mixing for a Study disclosed herein.

FIGS. 48A-B provide the appearance of dosage forms of a Study disclosed herein.

FIG. 49 provides the dispersion results for a Study disclosed herein.

FIGS. 50A-B show the appearance of dosage forms following heat stress of Study disclosed herein.

FIGS. 51A-B provide a summary of results of assay testing for a Study disclosed herein.

FIGS. 52A-B provide a summary of results of assay testing for a Study disclosed herein.

FIGS. 53A-D provide appearance of various batches of a Study disclosed herein.

FIGS. 54A-B provide a detailed breakdown of the formulation compositions of a Study disclosed herein.

FIG. 55 provides a flowchart for creating dosage forms of a Study disclosed herein.

FIG. 56 provides the preparation of the mixes for a Study disclosed herein.

FIGS. 57A-B provide the pH measurements that were taken for each batch at different stages of mix preparation for a Study disclosed herein.

FIGS. 58A-B provide the pH measurements that were taken for each batch at different stages of mix preparation for a Study disclosed herein.

FIGS. 59A-B provide the appearance observations during mixing for a Study disclosed herein.

FIGS. 60A-B show the appearance during mixing for a Study disclosed herein.

FIGS. 61A-D provide the appearance of dosage forms of a Study disclosed herein.

FIGS. 62A-D provide the appearance of dosage forms of a Study disclosed herein.

FIGS. 63A-B provide the dispersion results for a Study disclosed herein.

FIGS. 64A-G show the appearance of dosage forms following heat stress of Study disclosed herein.

FIG. 65 provides a detailed breakdown of the formulation compositions of a Study disclosed herein.

FIG. 66 provides a flowchart for creating dosage forms of a Study disclosed herein.

FIG. 67 provides the preparation of the mixes for a Study disclosed herein.

FIG. 68 provides the pH measurements that were taken for each batch at different stages of mix preparation for a Study disclosed herein.

FIG. 69 provides the pH monitored at various hold points for a Study disclosed herein.

FIG. 70 provides the appearance observations during mixing for a Study disclosed herein.

FIGS. 71A-F show the appearance during mixing for a Study disclosed herein.

FIG. 72 provides the microscopic appearance observations for Study disclosed herein.

FIGS. 73A-F show the appearance during mixing for a Study disclosed herein.

FIG. 74 shows the comparison of microscopy images prior and post homogenization for a Study disclosed herein.

FIG. 75 are the viscosity results for a Study disclosed herein.

FIG. 76 are the particle size results for a Study disclosed herein.

FIGS. 77A-C provide the appearance of dosage forms of a Study disclosed herein.

FIG. 78 provides the dispersion results for a Study disclosed herein.

FIG. 79 provides the results of the minimum stoppage/content uniformity of a Study disclosed herein.

### DETAILED DESCRIPTION

Disclosed herein are pharmaceutical compositions/formulations that include an Epinephrine hormone or salt or solvate thereof and methods of preparing these pharmaceutical compositions. Specifically, the present disclosure relates to freeze dried oral dispersible or disintegrating dosage forms that can preserve the stability of the Epinephrine hormone. The dosage forms can also be suitable for sublingual delivery.

As stated above, Applicants were able to develop a suspension-based epinephrine formulation capable of being used to form a freeze-dried dosage form. Specifically, Applicants determined that the inclusion of sodium metabisulfite and edetate disodium (“EDTA”) (this salt form of ethylenediaminetetracetic acid can provide enhanced stability) can protect the Epinephrine hormone or salt or solvate thereof from oxidative degeneration. This oxidative degeneration is explained in “Long-Term Stability Study of L-Adrenaline Injections: Kinetics of Sulfonation and Racemization Pathways of Drug Degradation” by Stepensky et al. in the Journal of Pharmaceutical Sciences, Vol. 93, No. 4, April 2004, which is hereby incorporated by reference in its entirety. In addition, Applicants discovered a formulating pH at which the vast majority or all of the API (i.e., the Epinephrine hormone or salt or solvate thereof) can be present in its solid (crystalline) form, in which it is considerably more stable both during the liquid/suspension formulation processing stages as well as the final dosage form. Lastly, Applicants discovered that using the lowest possible dosage form size to minimize the proportion of API in solution as a proportion of the finished product unit dose can minimize the surface area and potential for oxidative degeneration.

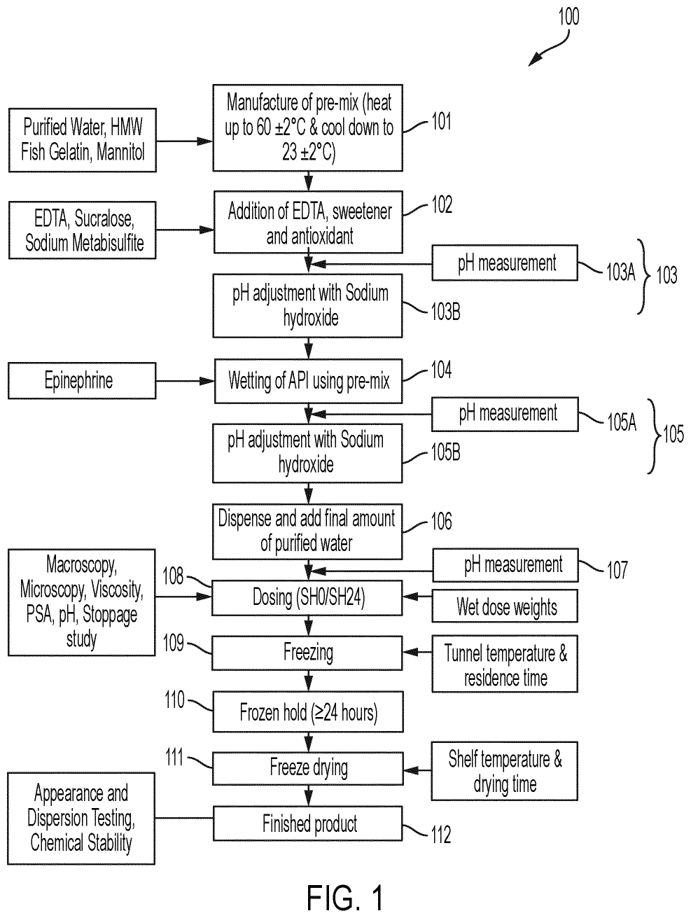

FIG. 1 illustrates a flow chart for method 100 of producing a dosage form that includes an Epinephrine hormone or salt or solvate thereof. At step 101, a pre-mix formulation can be formed.

The pre-mix formulation can be what helps provide the structure of the final dosage form. As such, the pre-mix matrix formulation can include a matrix former. The matrix former can provide the network structure of the dosage form that imparts strength and resilience during handling. Suitable matrix formers can include, without limitation, gelatin, pullulan, starch, or combinations thereof. Additional matrix formers can be found in EP 2624815 B1, which is herein incorporated by reference in its entirety. The gelatin can be fish gelatin, bovine gelatin, porcine gelatin, or combination thereof. Each of the gelatins can have different gelling characteristics. The extent a gelatin solution forms a gel can dependent on the concentration of the gelatin and the temperature of the gelatin solution. A solution of bovine gelatin tends to gel at temperatures of less than 18° C. and thus can be considered a gelling gelatin. In contrast, fish gelatin can remain in solution at temperatures as low as 5° C. and thus can be considered a non-gelling gelatin. In some embodiments, the gelatin can be a low endotoxin gelatin such as one sourced or one produced according to the process disclosed in U.S. application Ser. No. 16/295,223, which is hereby incorporated by reference in its entirety.

The temperature at which the pharmaceutical formulation is dosed can be as low as 5-18° C. As such, a formulation using bovine gelatin alone may not be dosed at these low temperatures. However, a combination of bovine gelatin and another type of gelatin (e.g., fish gelatin) can be used. Applicants discovered that fish gelatin can provide a freeze-dried tablet with robust matrix structure and a disintegration time of ≤10 seconds that is desirable to ensure dissolution of the API in saliva, such to allow sublingual absorption. In addition, the fish gelatin can provide freeze dried dosage forms of good physical attributes for formulation compositions that contain a high loading of soluble component like buffer salts, such as the amounts disclosed herein.

In some embodiments, the fish gelatin can be high molecular weight fish gelatin, standard molecular weight fish gelatin, or combinations thereof. High molecular weight fish gelatin is defined as a fish gelatin in which more than 50% of the molecular weight distribution is greater than 30,000 Daltons. Standard molecular weight fish gelatin is defined as fish gelatin in which more than 50% of the molecular weight distribution is below 30,000 Daltons.

The pre-mix formulation can also include a structure former. Suitable structure formers can include sugars including, but not limited to, mannitol, dextrose, lactose, galactose, cyclodextrin, or combinations thereof. The structure former can be used in freeze drying as a bulking agent as it crystalizes to provide structural robustness to the freeze-dried product.

The pre-mix formulation may also contain additional pharmaceutically acceptable agents or excipients. Such additional pharmaceutically acceptable agents or excipients include, without limitation, sugars, such as mannitol, dextrose, and lactose, inorganic salts, such as sodium chloride and aluminum silicates, gelatins of mammalian origin, fish gelatin, modified starches, preservatives, antioxidants, surfactants, viscosity enhancers, coloring agents, flavoring agents, pH modifiers, sweeteners, taste-masking agents, and combinations thereof. Suitable coloring agents can include red, black and yellow iron oxides and FD & C dyes such as FD & C Blue No. 2 and FD & C Red No. 40, and combinations thereof. Suitable flavoring agents can include mint, raspberry, licorice, orange, lemon, grapefruit, caramel, vanilla, cherry and grape flavors and combinations of these. Suitable pH modifiers can include citric acid, tartaric acid, phosphoric acid, hydrochloric acid, maleic acid, sodium hydroxide (e.g., 3% w/w sodium hydroxide solution), and combinations thereof. In some embodiments, the pre-mix formulation has an amount of a pH modifier to maintain a target pH of about 7-10, about 7.15-9.5, about 8-9, about 8.3-8.7, about 8.4-8.6, or about 8.5.

Suitable sweeteners can include, sucralose aspartame, acesulfame K and thaumatin, and combinations thereof. Suitable taste-masking agents can include a range of flavorings and combinations thereof. One of ordinary skill in the art can readily determine suitable amounts of these various additional excipients if desired.

The pre-mix formulation can also include a solvent. In some embodiments, the solvent can be water (e.g., purified water).

As stated above, a pre-mix formulation can be initially prepared by combining at least a matrix former, a structure former, and a solvent. In some embodiments, the pre-mix formulation can be prepared by dissolving a matrix former and a structure former in a solvent to form a premix. The premix can be heated to about 40-80° C., about 50-70° C., about 55-65° C., about 58-62° C., or about 60° C. The heat can be maintained for at least about 5 minutes, at least about 10 minutes, or at least about 15 minutes. In some embodiments, the heat is maintained for about 1-75 minutes, about 5-65 minutes, or about 10-60 minutes. The premix can then be cooled to about 10-30° C., about 15-30° C., about 20-26° C., about 21-25° C., or about 23° C.

Next, at step 102, a chelating agent, an antioxidant, and other excipients such as sweeteners can be added to the pre-mix formulation. In some embodiments, the chelating agent can be edetate disodium (“EDTA”). In some embodiments, the antioxidant is sodium metabisulfite. Other examples of antioxidants include butylated hydroxyanisole (BHA), butylated hydroxytoluene (BHT), propyl gallate (PG), Cysteine, and vitamin C, among others. In some embodiments, the sweetener is sucralose.

Due to observations drawn from evaluation of the previously discussed solution-based stability data, the suspension-based formulation included the highest level of antioxidant (e.g., sodium metabisulfite) assessed during development. In addition, research into components of other epinephrine injectable products such as Emerade pen identified edetate disodium as a potential means of enhancing stability. The EDTA is a chelating agent capable of acting synergistically with sodium metabisulfite.

At step 103, the pH of the pre-mix can be measured (103A) and adjusted (103B) to a target range of about 7-10, about 7.5-9.5, about 8-9, about 8.3-8.7, about. 8.4-8.6, or about 8.5. In some embodiments, the pH modifier to adjust the pH of the pre-mix can be sodium hydroxide, alkali metal hydroxides, alkaline earth metal hydroxides, citric acid, maleic acid, tartaric acid, and/or hydrochloric acid. Examples of alkali metal hydroxides can include sodium hydroxide, potassium hydroxide and mixtures thereof. One example of an alkaline earth metal hydroxide is magnesium hydroxide.

At step 104, the pre-mix can be added to the API or the API can be added to the pre-mix. In some embodiments, the API can be dispensed into a vessel (e.g., a stainless steel vessel) that can contain the final pharmaceutical formulation. In some embodiments, the pre-mix formulation can be used fraction by fraction to wet the API until all of the pre-mix is added to the vessel. In some embodiments, the API is an Epinephrine hormone. The API can include salts, esters, hydrates, solvates and derivatives of any of the foregoing active ingredients. Suitable derivatives are those that are known to skilled persons to possess the same activity as the active ingredient though the activity level may be lower or higher.

In some embodiments, the API is the Epinephrine hormone, derivatives or pharmaceutically acceptable salts or solvates thereof. In some embodiments, the API can be epinephrine base, or pharmaceutically acceptable salts or solvates thereof (e.g. Epinephrine maleate, Epinephrine tartrate, Epinephrine Bitartrate, Epinephrine hydrochloride, etc.).

In some embodiments, the formulation of pre-mix plus the API can be maintained at a temperature of about 5-25° C., 10-20° C., 13-17° C., or about 15° C. In some embodiments, the formulation of pre-mix plus the API can be maintained with constant stirring. In addition, after the API has been added to the mixture, the mixture can be protected from light for the remainder of the manufacture as the API may be sensitive to light.

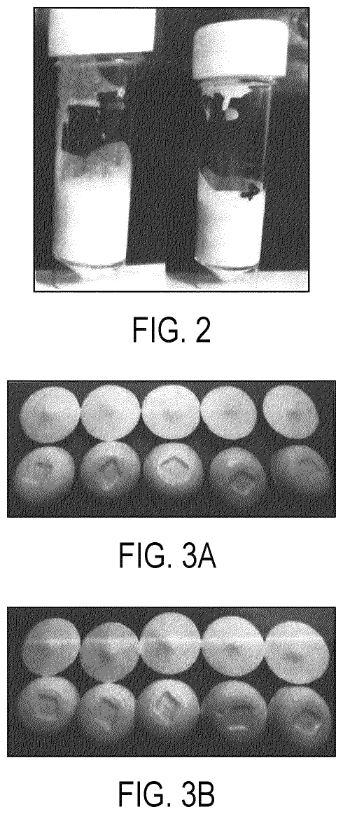

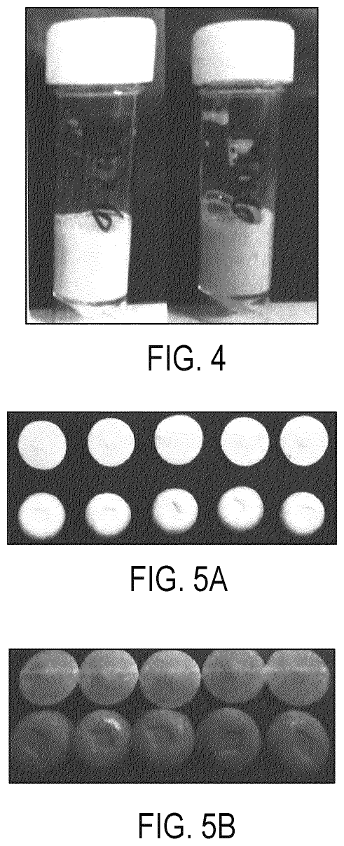

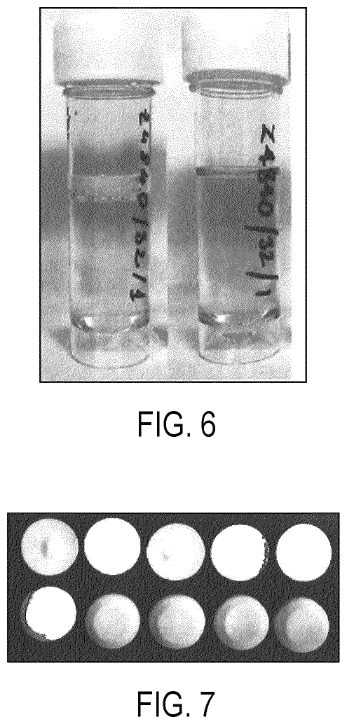

At step 105, the pH of the mixed formulation (i.e., pre-mix plus API) can be measured (105A) and adjusted (105B) to a target range of about 7-10, about 8-9, about 8.3-8.7, about 8.4-8.6, or about 8.5. In some embodiments, the pH modifier to adjust the pH of the mixed formulation can be sodium hydroxide, alkali metal hydroxides, alkaline earth metal hydroxides, citric acid, maleic acid, tartaric acid, and/or hydrochloric acid. In some embodiments, the pH modifier used to adjust the pH of the pre-mix (103B) can be used to adjust the pH of the mixed formulation (105). Applicants researched the material solubility at various pH levels and determined that at a pH of about 8.5, the majority or all of the API can be in its solid crystalline state and therefore expected to be stable. As such, the pH of the pre-mix formulation can be adjusted prior to the API addition to ensure minimal solubility and can be adjusted again post API addition, as required. FIG. 2 illustrates an example of the macroscopic appearance of a stable pharmaceutical suspension at 0-hour (left) and 24-hour (right) suspension hold. As you can see from the images, no discoloration was observed. FIG. 4 illustrates images of macroscopic appearance of unstable pharmaceutical suspension at 0-hour (left) and 24-hour (right) suspension hold. The unstable pharmaceutical suspensions of FIG. 4 were formulated with no sodium metabisulfite or EDTA and had a pH of 8.5. Clear discoloration was observed. FIG. 6 illustrates images of macroscopic appearance of unstable pharmaceutical solution formulated with sodium metabisulfite and a pH of 6 at 0-hour (left) and 24-hour (right) solution hold. Slight darkening and discoloration was visible.

At step 106, the final amount of solvent (e.g., purified water) required to obtain 100% batch size can be calculated, dispensed, and added to the mixed formulation to form the final pharmaceutical formulation. The pH of the final pharmaceutical formulation can then be measured as shown in step 107. The pharmaceutical formulation can be maintained at a temperature of about 5-25° C., 10-20° C., 13-17° C., or about 15° C. In some embodiments, the pharmaceutical formulation can be maintained with constant stirring.

The amount of components added at each step is specifically calculated to form the final pharmaceutical formulation. As used below, the pharmaceutical formulation refers to the pharmaceutical formulation prior to dosing and lyophilization. The pharmaceutical formulation can be a suspension. In some embodiments, the pharmaceutical formulation includes about 1-10 wt. %, about 2-8 wt. %, about 2-6 wt. %, about 3-5 wt. %, about 4-5 wt. %, or about 4.3-4.8 wt. % matrix former. In some embodiments, the pharmaceutical formulation can include at least about 1 wt. %, at least about 2 wt. %, at least about 3 wt. %, at least about 4 wt. %, at least about 4.3 wt. %, or at least about 4.8 wt. % matrix former. In some embodiments, the pharmaceutical formulation can include at most about 10 wt. %, at most about 8 wt. %, at most about 6 wt. %, at most about 5 wt. %, at most about 4.8 wt. %, or at most about 4.3 wt. % matrix former.

In some embodiments, the pharmaceutical formulation includes about 1-10 wt. %, about 1-7 wt. %, about 2-5 wt. %, about 2-4 wt. %, about 3-4 wt. %, or about 3.44-3.84 wt. % structure former. In some embodiments, the pharmaceutical formulation can include at least about 1 wt. %, at least about 2 wt. %, at least about 3 wt. %, at least about 3.44 or at least about 3.84 wt. % structure former. In some embodiments, the pharmaceutical formulation can include at most about 10 wt. %, at most about 7 wt. %, at most about 5 wt. %, at most about 4 wt. %, at most about 3.84 wt. %, or at most about 3.44 wt. % structure former.

In some embodiments, the pharmaceutical formulation includes about 0.1-1 wt. %, about 0.1-0.5 wt. %, about 0.2-0.4 wt. %, about 0.25-0.35 wt. %, or about 0.3 wt. % antioxidant. In some embodiments, the pharmaceutical formulation can include at least about 0.1 wt. %, at least about 0.2 wt. %, at least about 0.3 wt. %, at least about 0.4 wt. %, or at least about 0.5 wt. % antioxidant. In some embodiments, the pharmaceutical formulation can include at most about 1 wt. %, at most about 0.8 wt. %, at most about 0.6 wt. %, at most about 0.5 wt. %, at most about 0.4 wt. %, or at most about 0.3 wt. % antioxidant (sodium metabisulfite preferred).

In some embodiments, the pharmaceutical formulation includes about 0.01-0.1 wt. %, about 0.02-0.09 wt. %, about 0.03-0.08 wt. %, about 0.04-0.06 wt. %, about 0.045-0.055 wt. %, or about 0.05 wt. % chelating agent (e.g., EDTA). In some embodiments, the pharmaceutical formulation can include at least about 0.01 wt. %, at least about 0.02 wt. %, at least about 0.03 wt. %, at least about 0.04 wt. %, or at least about 0.05 wt. % chelating agent. In some embodiments, the pharmaceutical formulation can include at most about 0.1 wt. %, at most about 0.09 wt. %, at most about 0.08 wt. %, at most about 0.07 wt. %, at most about 0.06 wt. %, or at most about 0.05 wt. %.

In some embodiments, the pharmaceutical formulation includes about 0.1-1 wt. %, about 0.1-0.5 wt. %, about 0.2-0.4 wt. %, about 0.3-0.4 wt. %, or about 0.35 wt. % sweetener. In some embodiments, the pharmaceutical formulation can include at least about 0.1 wt. %, at least about 0.2 wt. %, at least about 0.3 wt. %, or at least about 0.35 wt. % sweetener. In some embodiments, the pharmaceutical formulation can include at most about 1 wt. %, at most about 0.8 wt. %, at most about 0.6 wt. %, at most about 0.5 wt. %, at most about 0.4 wt. %, or at most about 0.35 wt. % sweetener.

In some embodiments, the pharmaceutical formulation includes an amount of pH modifier (q.s.) such that the pH of the pharmaceutical formulation is about 740, about 8-9, about 8.3-8.7, about 8.4-8.6, or about. 8.5. In some embodiments, the pharmaceutical formulation includes about 1-10 wt. %, about 1-5 wt. %, about 1-3 wt. %, about 2-3 wt. %, or about 2.51-2.57 wt. % pH modifier. In some embodiments, the pH modifier can be a pH modifier solution. For example, in some embodiments, the pH modifier in the pharmaceutical formulation can be a 5% sodium hydroxide solution. In this example, 95% of this solution (i.e., the water) is removed during freeze drying. Accordingly, the pH modifier in the dosage form would be the pH modifier itself even if the pH modifier started as a pH modifier solution.

In some embodiments, the pharmaceutical formulation includes an amount of solvent such that the pharmaceutical formulation is q.s. 100%.

In some embodiments, the pharmaceutical formulation can include about 1-30 wt. %, about 1-25 wt. %, about 1-20 wt. %, about 1-15 wt. %, 3-14 wt. %, or about 3.33-13.33 wt. % API (e.g., solid (crystalline) epinephrine). In some embodiments, the pharmaceutical formulation includes at least 1 wt. %, at least 2 wt. %, at least 3 wt. %, at least 3.33 wt. %, at least 13.33 wt. %, at least 15 wt. %, or at least 20 wt. % API. In some embodiments, the pharmaceutical formulation includes at most about 30 wt. %, at most about 25 wt. %, at most about 20 wt. %, at most about 15 wt. %, or at most about 13.33 wt. % API.

In some embodiments, the viscosity of the pharmaceutical formulations can be about 1-30 mPas, about 5-20 mPas, or about 9-17 mPas. The viscosity can be measured with a Haake VT550 viscometer with readings provided at a shear rate of 500 s⁻¹. In some embodiments, the particle size of the pharmaceutical formulation can have a D10 of about 1-20 microns, a D50 of about 10-40 microns, and a D90 of about 30-160 microns.

In some embodiments, the API is included in the dosage forms disclosed herein in an amount, which is sufficient to render it pharmaceutically effective when provided in a dosage form. A person of skill in the art can readily determine the pharmaceutically effective amount for a given disease or infection based on, among other facts, age and weight of the patient to whom the dosage form will be administered. In addition, the BP pharmaceopeia specification for an adrenaline injection specifies the following:
Content of adrenaline: 0.09 to 0.11% w/v, and not less than 0.085% w/v is L-Adrenaline (equates to 90 to 110% label claim for tablet formulation)
D-Adrenaline: Not more than 15%
Impurity F (Epinephrine Sulfonate): Not more than 15%
Impurity B (Norepinephrine): Not more than 1%
Unknown Individual Impurity: Not more than 0.5%
Total of Unknown Individual Impurities: Not more than 1%
Total of all impurities including D-Adrenaline: Not more than 16.0%

As such, the pharmaceutical formulations and dosage forms containing Epinephrine API are considered stable if they meet the above pharmaceopeia specifications. The example pharmaceutical formulations and dosage forms disclosed herein were targeted to the above analytical limits for impurities. In some embodiments, the dosage form can be considered stable if it does not exceed the specified degradation limits above with no discoloration/adverse change in unit appearance throughout the specified product shelf life. In some embodiments, a pharmaceutical formulation can be considered stable if it does not exceed the specified degradation limits and does not yield any results indicative of instability during in-process observations and testing (i.e., macroscopic discoloration). Oxidative degradation of the API (to form adrenochrome for example) can impart a pink/brown discoloration. Thus, a similar change in macroscopic appearance of the formulation or dosage form (from white to pink/brown) can indicate a lack of stability.

At step 108 of FIG. 1, the pharmaceutical formulation can be dosed into a preformed mold. As used herein, “dosed” (or similar terminology) refers to the deposition of a pre-determined aliquot of solution or suspension. As used herein, “preformed mold” refers to any suitable container or compartment into which an aqueous solution or suspension may be deposited and within which subsequently freeze dried. In certain embodiments of the present disclosure, the preformed mold is a blister pack with one or more blister pockets. Predetermined aliquots in an amount of about 150-1000 mg or about 500 mg wet filling dosing weight of the pharmaceutical formulation can be metered into preformed molds. The minimum unit size (wet fill weight, 150 mg) can be selected to minimize the amount of API in solution proportionally to the unit dose, and therefore its surface area and potential for oxidative degradation in the final dosage form.

At step 109, the dosed pharmaceutical formulations can be frozen in the preformed molds. The dosed formulations in the preformed molds can be frozen by any means known in the art. For example, the formulations can be passed through a cryogenic chamber (e.g., liquid nitrogen tunnel). The temperature during freezing can be between about −40 to −120° C., about −60 to −80° C., about −65 to −75° C., or about −70° C. The freezing duration can range from about 1.5-5 minutes, about 2-4.5 minutes, about 2.5-4 minutes, about 3-4 minutes, about 3-3.5 minutes, or about 3.25 minutes. For example, the dosed formulation can be frozen at −70° C. with a residence time of 3 mins and 15 seconds.

At step 110 of FIG. 1, the frozen units in the preformed molds can be collected and placed in a freezer at a temperature of less than about −25° C., about −20° C., about −15° C., about −10° C., about −5° C. prior to freeze drying.

The frozen held units can be freeze-dried in step 111 to form the dosage forms of step 112. During the freeze-drying process, the water is sublimated from the frozen units. In some embodiments, the frozen units can be loaded onto the shelves of a freeze-drier. In some embodiments, the freeze-drier shelves can be precooled to below −25° C. Once the frozen units are in the freeze-drier, the freeze-drying cycle can be initiated. In some embodiments, a vacuum can be pulled and the shelf temperature raised once the freeze-drying cycle is initiated. The freeze-drier can operate at low pressure (i.e., vacuum). In some embodiments, the freeze-drier can operate at a pressure of about less than or equal to 1000 mbar, about 900 mbar, about 800 mbar, about 700 mbar, about 600 mbar, about 500 mbar, or about 400 mbar. The freeze-drying step can include holding the frozen units at the above the precooled temperatures (i.e., about −15 to 15° C., about −10 to 10° C., about −5 to 5° C., or about 0° C.) for about 1-10 hours, about 2-8 hours, about 4-8 hours, about 5-7 hours, or about 6 hours. In some embodiments, the units are freeze-dried at about 0° C. for about 6 hours.

The freeze-dried dosage forms can be removed from the freeze-drier and inspected for any defects. FIGS. 3A-3B illustrate images of stable finished dosage form with sodium metabisulfite and EDTA appearance (product dosed at 0-hour (3A) and 24-hour (3B) suspension hold time. No discoloration was observed. FIGS. 5A-5B illustrate images of unstable finished dosage form appearance (product dosed at 0-hour (5A) and 24-hour (5B) suspension hold time. Clear discoloration was observed. The unstable dosage form of FIGS. 5A-5B was not formulated with sodium metibisulfate or EDTA with a pH of 8.5. FIG. 7 illustrate images of unstable finished dosage form appearance (product dosed at 0-hour solution suspension). The unstable finished dosage form of FIG. 7 was formulated with sodium metabisulfite and a pH of 6.0. No obvious discoloration was observable. However, the product was dosed immediately after completion of mix (i.e., minimal solution hold period) and chemical stability data obtained during storage and in-process finished product studies was poor (see Table 5).

On completion of the freeze drying cycle, the dosage forms can be sealed (e.g., lidding foil applied to blister).

The water in the freeze-dried dosage forms can be removed via sublimation during freeze-drying. Accordingly, the dosage form (i.e., post lyophilization) can include about 5-55 wt. %, about 10-50 wt. %, about 10-45 wt. %, about 10-40 wt. %, about 15-40 wt. %, or about 19.63-37.48 wt. % matrix former. In some embodiments, the dosage form can include at least about 10 wt. %, at least about 15 wt. %, at least about 19.63 wt. %, at least about 20 wt. %, at least about 25 wt. %, or at least about 30 wt. % matrix former. In some embodiments, the dosage form can include at most about 55 wt. %, at most about 50 wt. %, at most about 45 wt. %, at most about 40 wt. %, at most about 37.48 wt. %, at most about 35 wt. %, at most about 30 wt. %, at most about 25 wt. %, at most about 20 wt. %, or at most about 15 wt. % matrix former.

In some embodiments, the dosage form (i.e., post lyophilization) includes about 10-40 wt. %, about 10-35 wt. %, about 15-30 wt. %, or about 15.70-29.98 wt. % structure former. In some embodiments, the dosage form can include at least about 5 wt. %, at least about 10 wt. %, at least about 15 wt. %, at least about 15.70 wt. %, at least about 20 wt. %, or at least about 25 wt. % structure former. In some embodiments, the dosage form can include at most about 40 wt. %, at most about 35 wt. %, at most about 30 wt. %, at most about 29.98 wt. %, at most about 25 wt. %, or at most about 20 wt. % structure former.

In some embodiments, the dosage form (i.e., post lyophilization) includes about 0.1-5 wt. %, about 0.5-5 wt. %, about 0.5-4 wt. %, about 0.5-3 wt. %, about 1-3 wt. %, about 1-2.5 wt. %, or about 1.37-2.34 wt. % antioxidant. In some embodiments, the dosage form can include at least about 0.1 wt. %, at least about 0.5 wt. %, at least about 1 wt. %, at least about 1.37 wt. %, or at least about 2 wt. % antioxidant. In some embodiments, the dosage form can include at most about 5 wt. %, at most about 4 wt. %, at most about 3 wt. %, at most about 2.5 wt. %, at most about 2.34 wt. %, at most about 2 wt. %, or at most about 1.5 wt. % antioxidant.

In some embodiments, the dosage form (i.e., post lyophilization) includes about 0.1-1 wt. %, about 0.15-0.5 wt. %, about 0.2-0.5 wt. %, or about 0.24-0.42 wt. % chelating agent. In some embodiments, the dosage form can include at least about 0.1 wt. %, at least about 0.15 wt. %, at least about 0.2 wt. %, at least about 0.24 wt. %, at least about 0.3 wt. %, or at least about 0.5 wt. % chelating agent. In some embodiments, the dosage form can include at most about 1 wt. %, at most about 0.5 wt. %, at most about 0.42 wt. %, at most about 0.4 wt. %, at most about 0.35 wt. %, or at most about 0.35 wt. %.

In some embodiments, the dosage form (i.e., post lyophilization) includes about 0.1-5 wt. %, about 0.5-2 wt. %, about 1-3 wt. %, or about 1.61-2.76 wt. % sweetener. In some embodiments, the dosage form can include at least about 0.1 wt. %, at least about 0.5 wt. %, at least about 1 wt. %, at least about 1.61 wt. %, or at least about 2 wt. % sweetener. In some embodiments, the dosage form can include at most about 5 wt. %, at most about 3 wt. %, at most about 2.76 wt. %, at most about 2.5 wt. %, at most about 2 wt. %, or at most about 1.61 wt. % sweetener.

In some embodiments, the dosage form includes about 0.1-5 wt %, about 0.1-3 wt. %, about 0.1-2 wt. %, about 0.1-1.5 wt. %, about 0.2-1.5 wt. %, about 0.3-1.2 wt. %, about 0.5-1.1 wt. %, about 0.5-1 wt. %, or about 0.58-0.99 wt. % pH modifier. In some embodiments, the dosage form can include at least about 0.1 wt. %, at least about 0.2 wt. %, at least about 0.3 wt. %, at least about 0.4 wt. %, at least about 0.5 wt. %, at least about 0.58 wt. %, at least about 0.75 wt. %, or at least about 1 wt. % pH modifier. In some embodiments, the dosage form can include at most about 5 wt. %, at most about 3 wt. %, at most about 2 wt. %, at most about 1.5 wt. %, at most about 1.25 wt. %, at most about 1 wt. %, at most about 0.99 wt. %, or at most about 0.75 wt. %, pH modifier. As stated above, the pH modifier in the dosage form is just the pH modifier itself even if the pH modifier was in solution in the pharmaceutical formulation. The freeze-drying process can remove the water from the pH modifier solution.

In some embodiments, the dosage form (i.e., post lyophilization) can include about 10-75 wt. %, about 10-70 wt. %, about 15-70 wt. %, about 20-65 wt. %, about 25-65 wt. %, or about 26.03-60.86 wt. % API (e.g., epinephrine). In some embodiments, the dosage form includes at least 5 wt. %, at least 10 wt. %, at least 15 wt. %, at least 20 wt. %, at least 25 wt. %, at least 26.03 wt. %, or at least 30 wt. % API. In some embodiments, the dosage form includes at most about 80 wt. %, at most about 75 wt. %, at most about 70 wt. %, at most about 65 wt. %, at most about 60.86 wt. %, at most about 60 wt. %, at most about 55 wt. %, or at most about 50 wt. % API.

The dosage forms of the present disclosure can be dissolving dosage forms and accordingly have the distinct advantage of a faster disintegrating time.

### Formulations, Test Methods, and Examples

As discussed above, following the inability to achieve suitable stability for a freeze-dried, solution-based epinephrine product, the formulation strategy was altered to target a freeze-dried, suspension-based epinephrine product. The following were the studies conducted on the solution-based formulations and the suspension-based formulations.

Solution-Based Formulation Study 1

The objective of this study was to evaluate the technical feasibility of developing a 20 mg Epinephrine formulation. The incorporation of Epinephrine into two different matrixes (HMW fish gelatin and non-ox bovine gelatin) was to be assessed in order to ascertain which was to be the more suitable formulation for this product. Three different unit wet fill weights were assessed (500 mg, 750 mg and 1000 mg) in order to assess the effects of the API concentration in the formulation on the finished units; critical physical attributes of the dried units were to be evaluated for appearance and dispersion time.

In order to study the feasibility of incorporating Epinephrine into solution-based formulations, five batches were to be produced with the aim of:
Identifying the most suitable gelatin type for the formulation: fish gelatin (low temperature dosing) or bovine gelatin (ambient temperature dosing)
Assessing the incorporation of Epinephrine into the formulation in terms of particle size, if not all in solution
Assessing the solubility of the API at pH 3.5±0.2, and the stability of the mix
Assessing up to three different wet fill weights, and two different matrix types
Assessing the impact of suspension/solution hold time on the finished product
Assessing the inclusion of an antioxidant (sodium metabisulphite, 0.05% w/w)
Assessing the effect of light protection (e.g. with UV light filters and aluminium foil) on potential oxidation of the mix

The mixes were to be dosed at approximately 24 and 48 hours after completion of the mixing stage (24 and 48 hours solution hold; SH24 and SH48). The products were to have ≥12 hours frozen hold (FH) prior to freeze drying. The batches were to be freeze dried at a shelf temperature of 0° C. on an 18 hour cycle. Appearance and dispersion testing were to be performed on all batches.

The following Table 1 is the formulations used in the Solution-Based Formulation Study 1:

TABLE 1

| | | | | | Sodium | Unit | pH (citric |
|---|---|---|---|---|---|---|---|
| | Gelatin | Gelatin | Mannitol | Epinephrine | metabisulphite | wet fill | acid for |
| Batch | Type | % w/w | % w/w | % w/w | % w/w | (mg) | adjustment) |
| 1 | Fish | 5.00 | 4.00 | 4.00 | 0.05 | 500 | 3.50 ± 0.2 |
| | (HMW) | | | | | | |
| 2 | Fish | 5.00 | 4.00 | 2.00 | N/A | 1000 | |
| | (HMW) | | | | | | |
| 3 | Bovine | 4.00 | 3.00 | 4.00 | N/A | 500 | |
| 4 | Bovine | 4.00 | 3.00 | 2.67 | N/A | 750 | |
| 5 | Bovine | 4.00 | 3.00 | 2.00 | 0.05 | 1000 | |

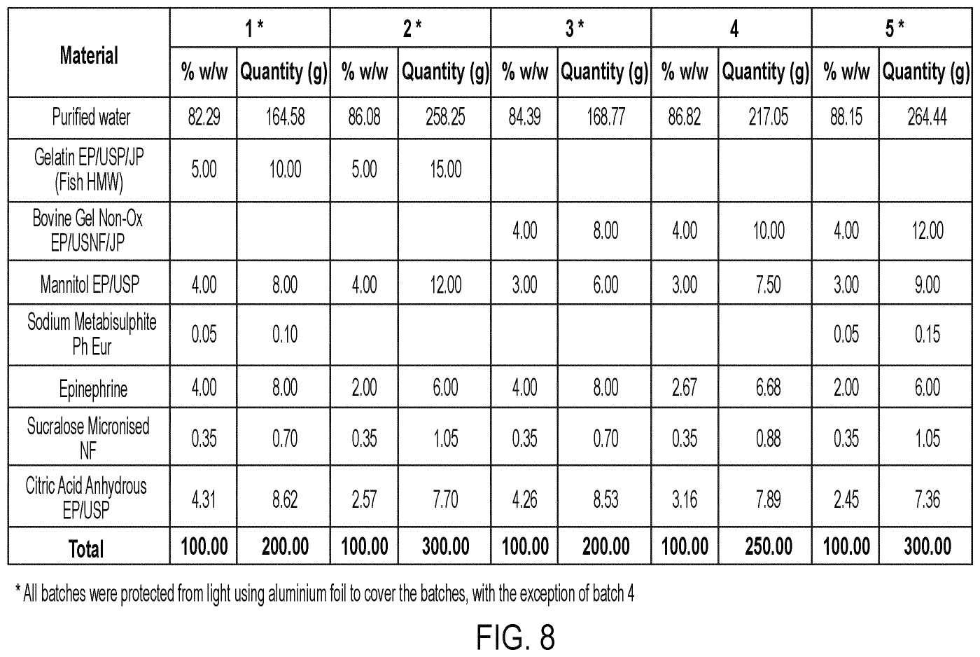

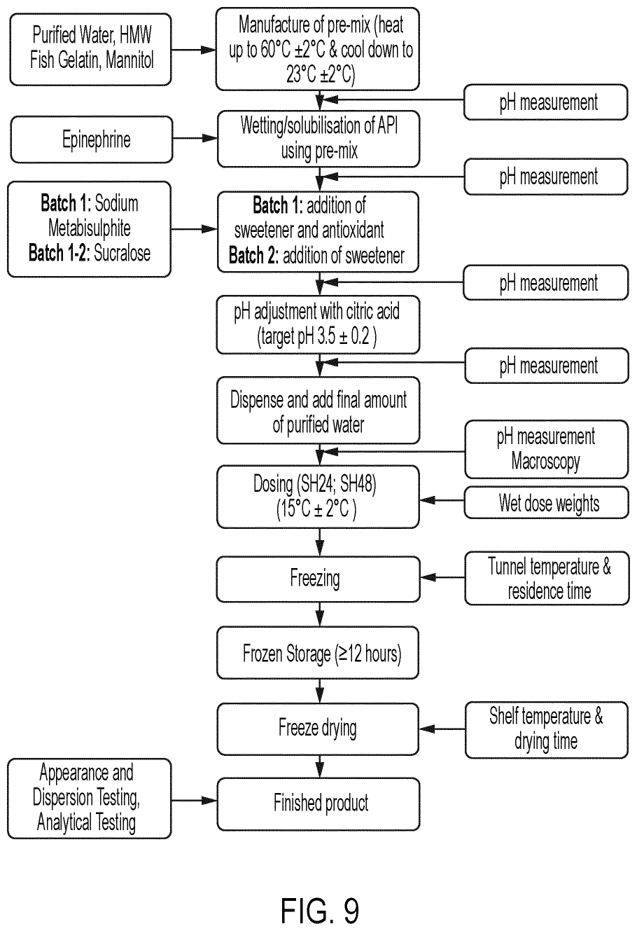

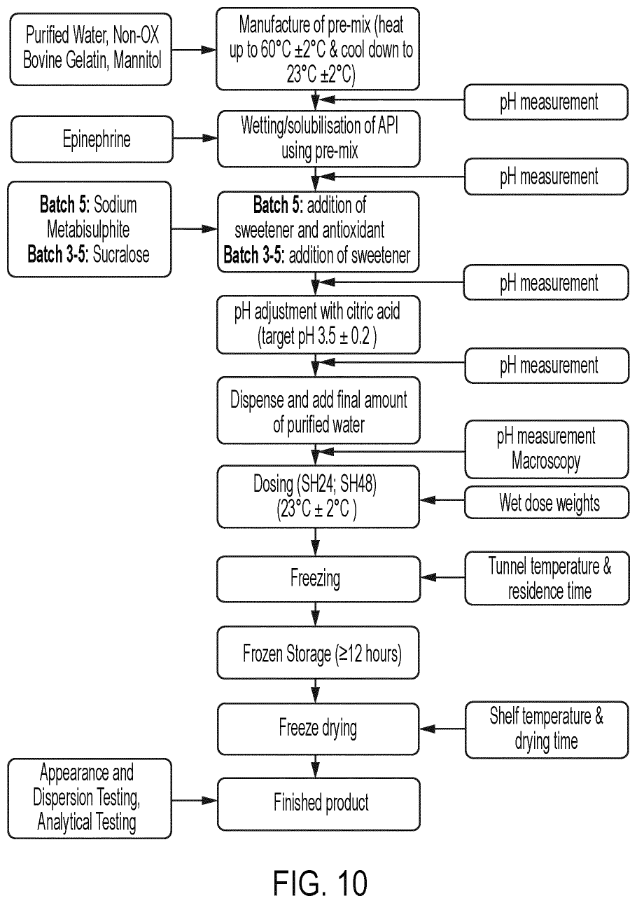

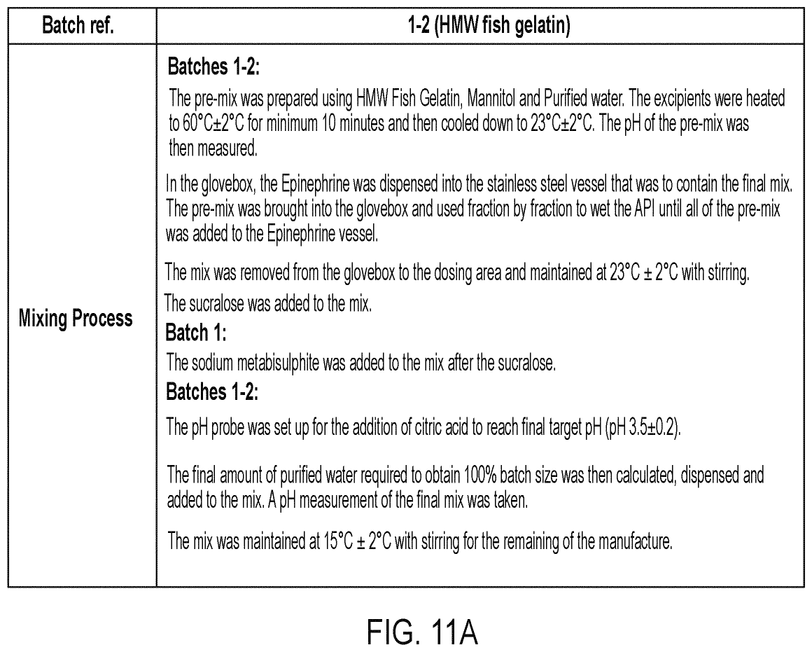

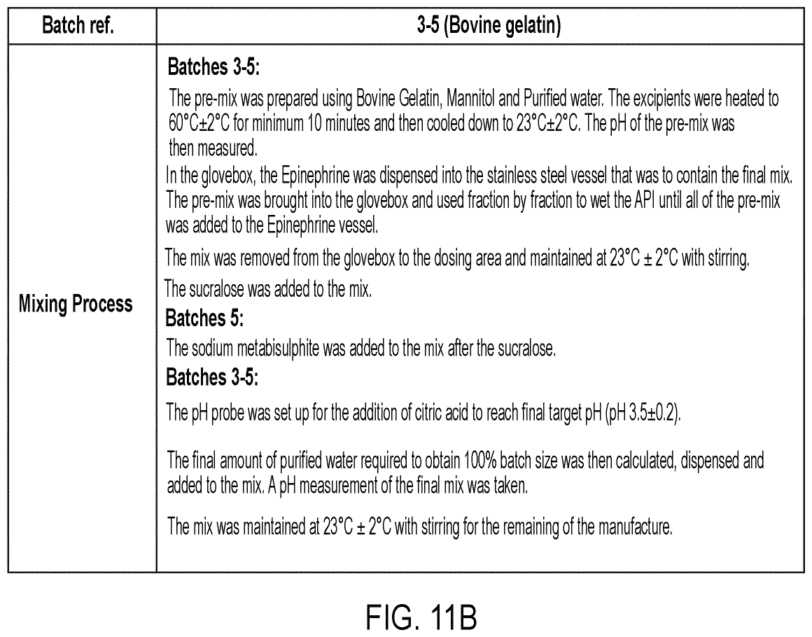

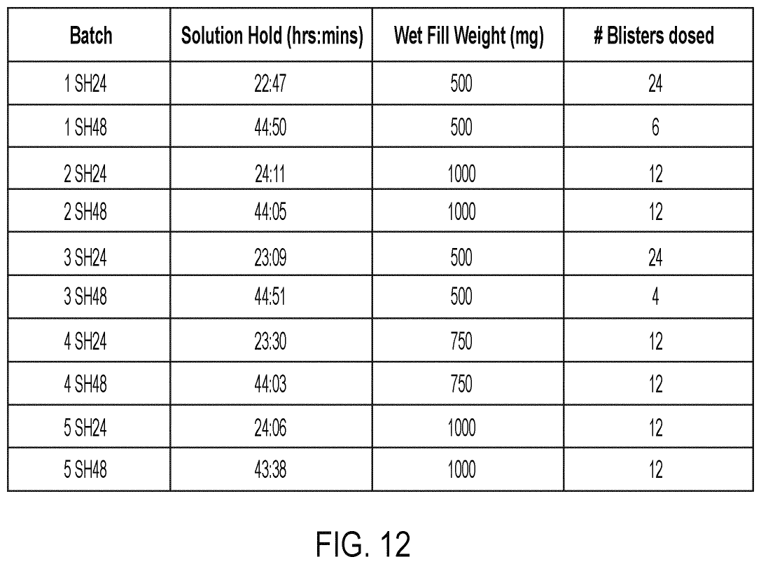

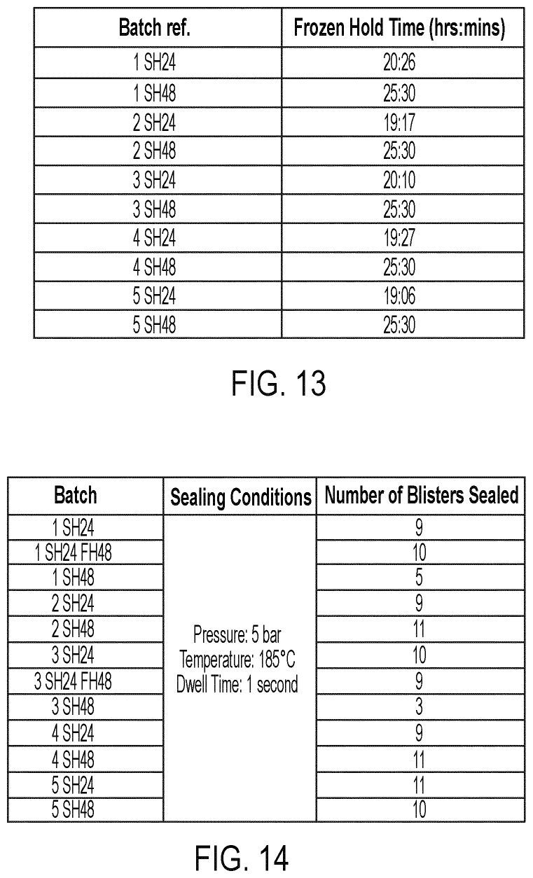

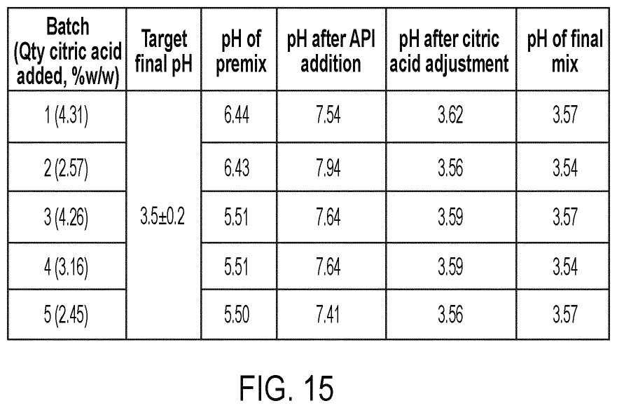

More details of the specific formulations can be found in FIG. 8. The flowchart for creating the dosage forms in Solution-Based Formulation Study 1 can be found in FIGS. 9-10. The preparation of the mixes for Solution-Based Formulation Study was conducted as Described in FIGS. 11A-B. FIG. 12 provides a table summarizing the dosing points, wet fill weights of the batches, and the number of blisters dosed per batch. The trays were frozen at −90° C. with a freeze tunnel residence time of 3:15 min. The time period from when the frozen product was transferred from the freeze tunnel to a freezer and stored below −15° C. (freezer set up at approximately −25° C.) until loading into the freeze dryer is referred to as the frozen hold (FH) time. The batches had a frozen hold of greater than or equal to 12 hours. The range of frozen holding times for this study is shown in FIG. 13. Batches were dried at 0° C. for 18 hours. Two dryer loads were required as there were two dosing time-points. The parameters used for sealing and number of blisters sealed for each batch can be found in FIG. 14. The pH measurements that were taken for each batch at different stages of mix preparation are detailed in FIG. 15. A noticeable increase in pH was observed following the addition of the API to the mixes. It was noted for Batch 1 that pH adjustment with citric acid caused the mix to transition from a suspension, white in color, to a clear solution. Following adjustment with citric acid, no significant change in pH was observed after the addition of the final amount of purified water.

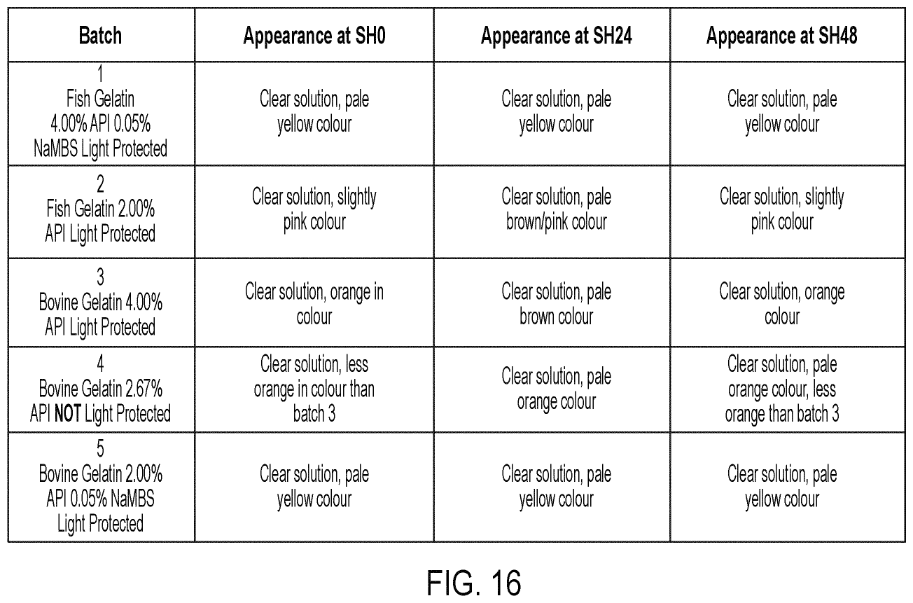

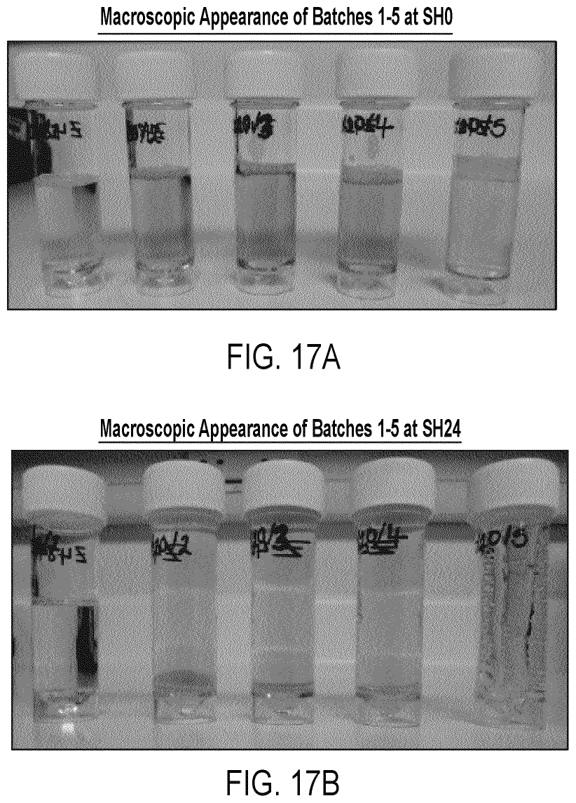

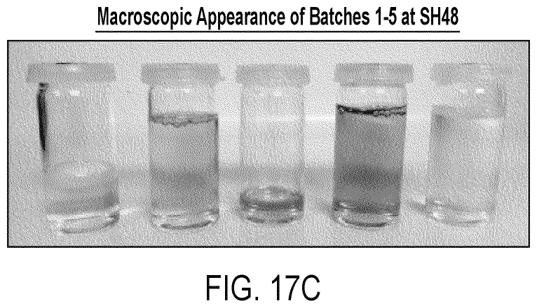

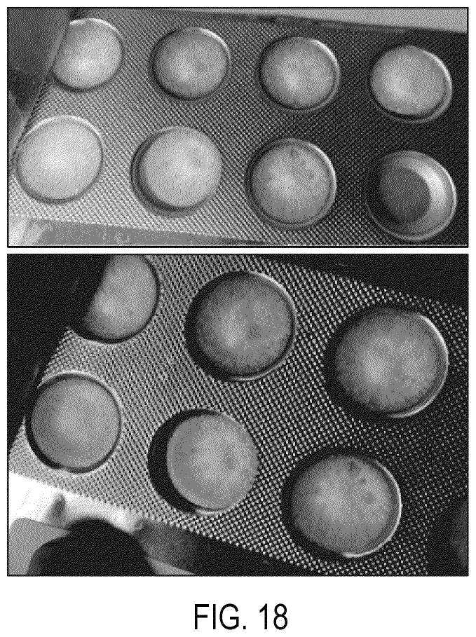

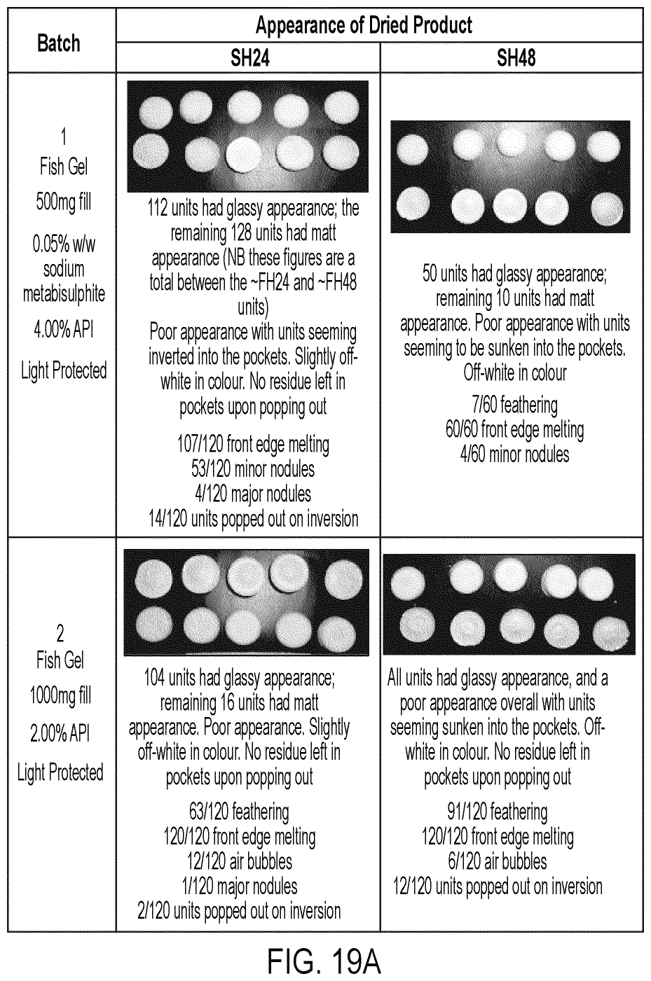

The appearance of the solutions was recorded at the end of mixing, after approximately 24 hours stirring and after approximately 48 hours stirring. The observations recorded for each mix are summarized in FIG. 16 and illustrated in FIGS. 17A-C. From the macroscopic appearance of batches 2-4, it is evident that some oxidation of the Epinephrine occurred as the mix was discolored compared to batches 1 and 5, which contained the antioxidant sodium metabisulphite and appear not to have oxidized or not oxidized much as they are mostly colorless. The macroscopic appearance of all five mixes appeared to stay fairly constant over the solution hold time.

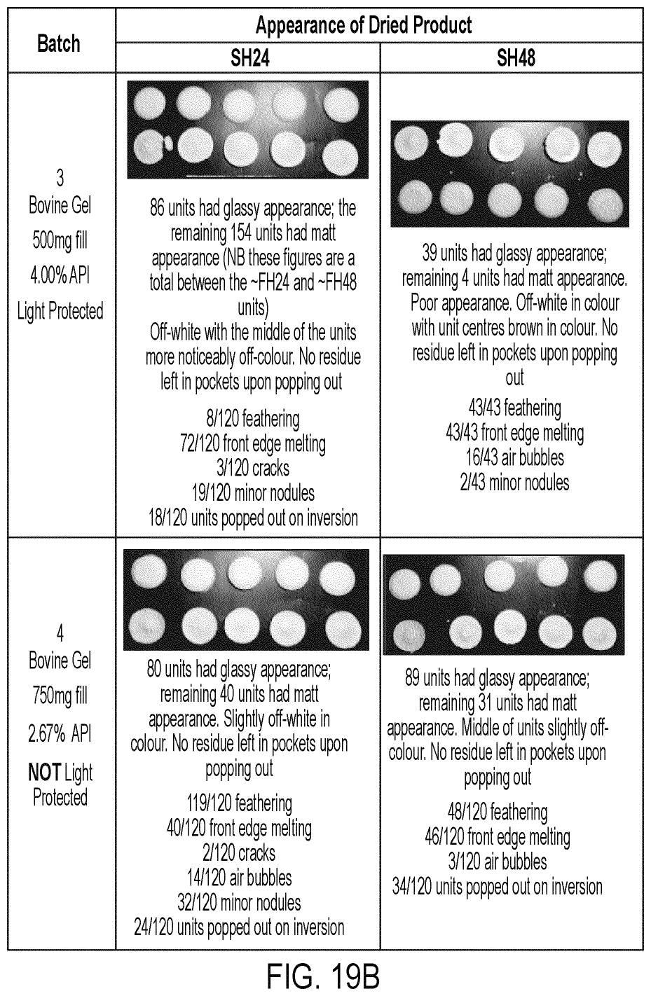

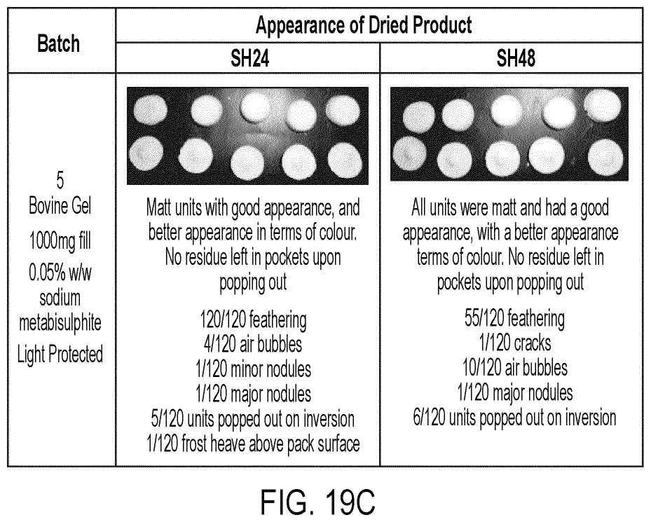

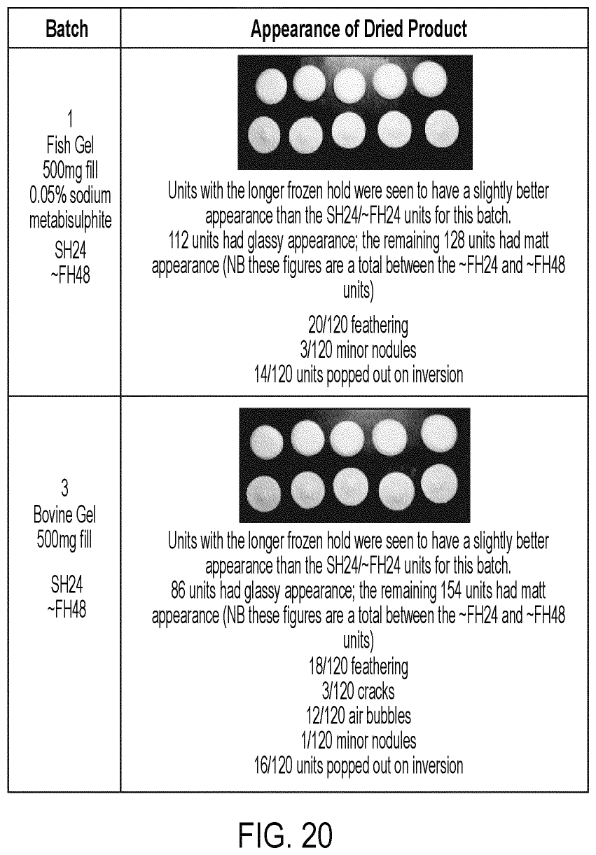

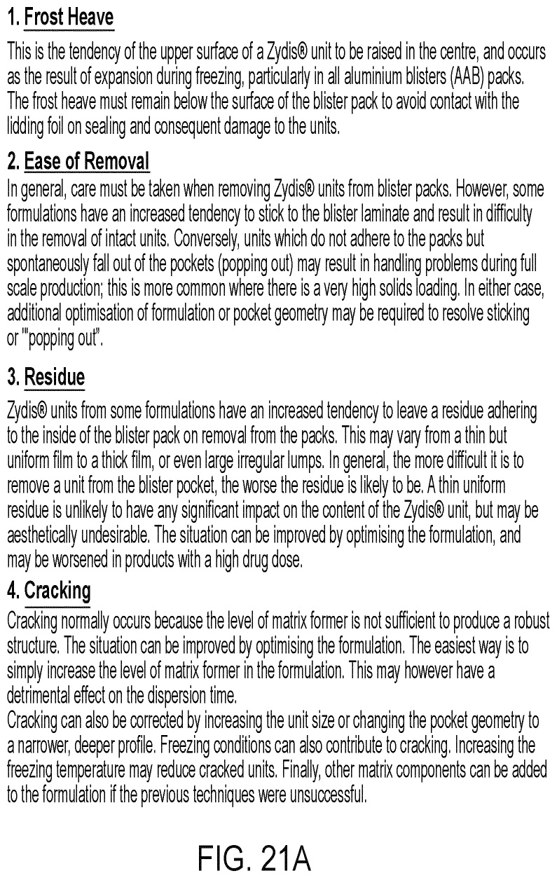

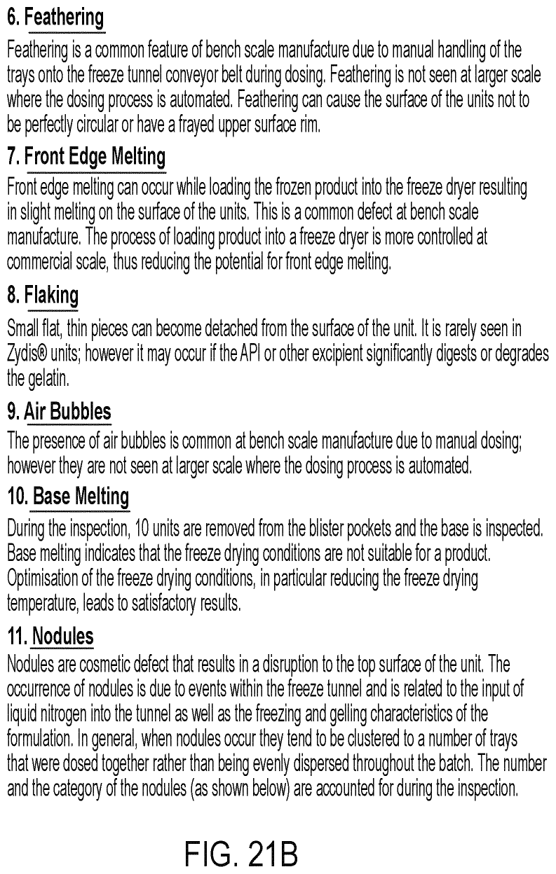

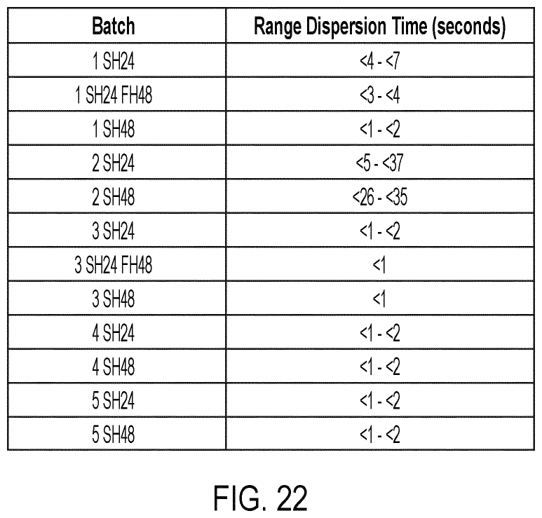

All batches were inspected and most units produced were round, off-white units with overall poor appearance and many exhibiting front edge melting and a glassy appearance, which was noted to differ even between units from the same blister. A reference picture for what is meant by “glassy” can be seen in FIG. 18. FIGS. 19A-C include the appearance of the freeze-dried products from batches 1-5. Extra blisters for batches 1 and 3 were placed under a longer frozen hold ˜FH48. These units were dried in the first dryer load, and the finished product observations are shown in FIG. 20. The terms used to describe the unit appearance are in FIGS. 21A-B. The dispersion time results for batches 1-5 are summarized in FIG. 22.

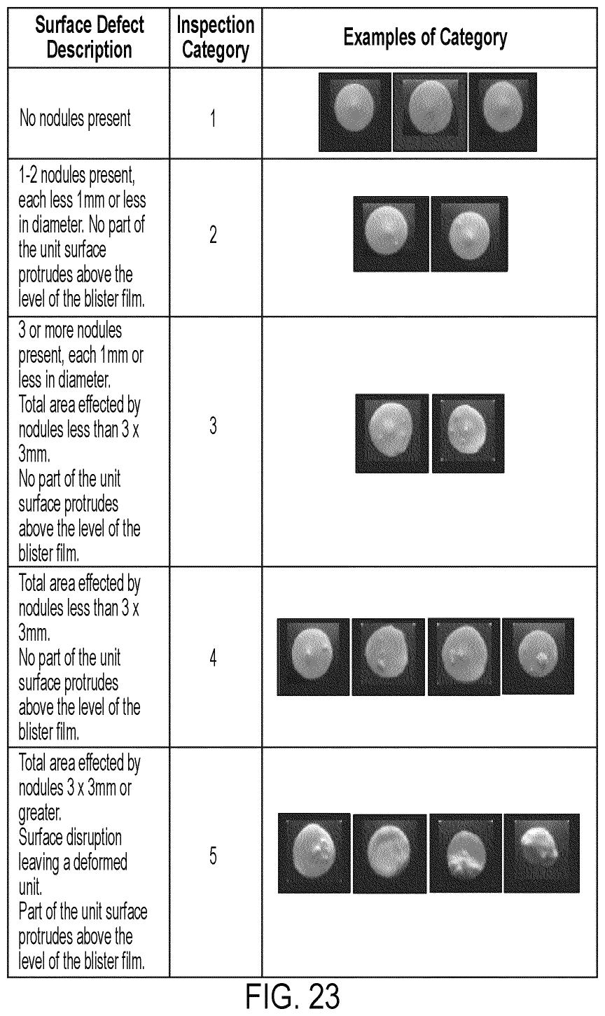

The dispersion time is a measurement of the time required for a unit to fully wet. The dispersion time is an in-process test. a minimum of 5 tablets were tested. First, a beaker is prepared containing approximately 200 mL of purified water at 20° C.±0.5° C. Each tablet is then removed from the blister package and the tablet is placed base down on the surface of the water. The time is taken for the time each tablet takes to fully wet or dissociate. Wetting=the time taken for the unit to fully wet. The wetting of the tablet can occur in patches, eventually merging together so that the whole unit is wet. The dispersion test is considered complete when the center of the unit is a wetted mass. Thus, take the wetting time from when the center of the unit has wetted through as this is the thickest part of the unit. Record the wetting time for each of the five tablets. The criterion for the dispersion characteristic test is if the 5 tablets can be fully wetted and/or dissociated into a palpable mass without the presence of hard lumps and skin in 10 seconds or less. In some embodiments, the dosage forms disclosed herein can be fully wetted and/or dissociated into a palpable mass without the presence of hard lumps and/or skin in 10 seconds or less. The inspection categories and examples of each category can be found in FIG. 23 along with a description of the surface defect for each inspection category. The acceptable criteria is circular tablets that can be removed from packaging without breaking and are generally free of defects.

Batch 1 units dispersed in less than 7 seconds, which is within the acceptable criterion of less than or equal to 10 seconds. Batches 3-5 all dispersed in less than 2 seconds, which is also within the acceptable criterion. Units for batch 2 had mean dispersion time s of 22.53 seconds and 30.10 seconds for SH24 and SH48, respectively, which did not meet acceptable criterion.

Sealed batches from batches 1, 3, and 5 underwent a heat stress study, where the blisters were stored at 60° C. for up to 21 days in order to emulate longer-term stability testing conditions. Units were removed from the chamber every 7 days and their appearance was recorded. Only batch 5 from this study was assayed at every time-point of the analysis. The results of these studies were to be used in order to influence future formulation factors such as proportion of antioxidant (i.e. sodium metabisulphite).

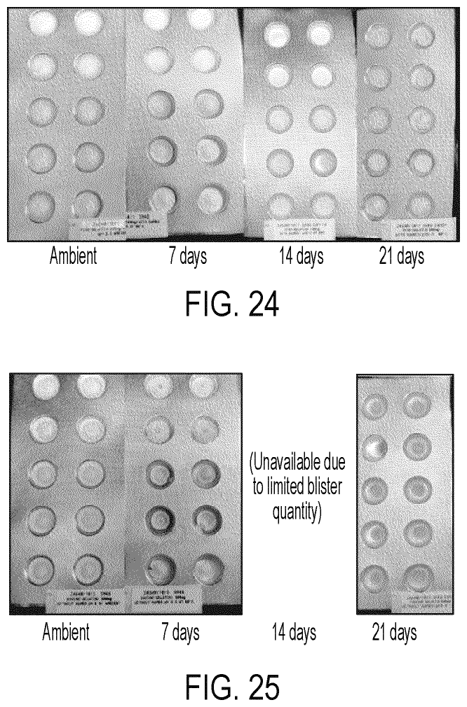

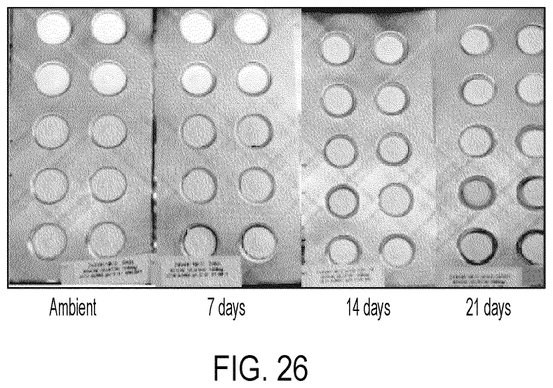

FIG. 24 shows the appearance of units from batch 1 following heat stress. The unit structure of batch 1 was unacceptable at all time-points, and there was evident color change and shrinkage visible upon storage. These units were not assayed. FIG. 25 shows the appearance of units from batch 3 following heat stress. The unit structure of batch 3 was unacceptable for all time-points and there was evident color change and shrinkage upon storage. The units were assayed at Day 0, but not for the remainder of the heat stress study. The mean assay recovery—ambient (%) of Batch 3 units on Day 0 was 102.6. At ambient conditions without heat stress, the mean assay recovery was within the 95-105% label claim target for this product. The heat-stressed units were not assayed, however it is evident from the observed color change that there is a degree of oxidation occurring under the aggressive storage conditions, which is potentially exacerbated by the lack of antioxidant in the formulation. FIG. 26 shows the appearance of units from batch 5 following heat stress. Visually the units from this batch had acceptable unit structure at the initial time-point, however, there is slight color change and shrinkage evident over the course of the study. The unit appearance after 21 days of heat stress is better than the other batches tested from this study. The following Table 2 is the assay data for batch 5:

TABLE 2

Mean Assay Recovery (%)

| Sample | Ambient | 60° C. |
|---|---|---|
| Batch 5, Day 0 | 102.2 | N/A |
| Batch 5, Day 7 | 98.7 | 90.4 |
| Batch 5, Day 14 | 102.4 | 82.7 |
| Batch 5, Day 21 | 100.6 | 76.0 |

The ambient samples remain within the 95-105% assay recovery limits over the 21 day study period, however the assay recovery for the 60° C. samples falls significantly over the 21 day study period, suggesting this formulation is not well protected against oxidation and may not have a suitable shelf life. The heat stress study results reported here have indicated that sodium metabisulphite can provide an antioxidant effect. The difference between bovine and fish gelatin appears inconclusive so far and both options should continue to be considered.

Solution-Based Formulation Study 2

The objective of this study was to evaluate the technical feasibility of developing an Epinephrine formulation that competes with the current EpiPen product. The incorporation of the API into two different matrixes (HMW fish gelatin and non-ox bovine gelatin) was to be assessed in order to ascertain which was to be the more suitable formulation for this product. Three different unit wet fill weights were assessed (500 mg, 750 mg and 1000 mg) in order to assess the effects of the formulation on the finished units; critical physical attributes of the dried units were to be evaluated for appearance and dispersion time. Citric acid was to be used as a pH modifier, and the pH at which the API went into solution was also assessed.

In order to study the feasibility of incorporating Epinephrine into solution-based formulations for dosage forms, six batches were to be produced with the aim of:
Identifying the most suitable gelatin type for the formulation: fish gelatin (low temperature dosing) or bovine gelatin (ambient temperature dosing)
Investigating further the pH at which the API goes into solution and assessing stability of the mix over time
Taking forward sodium metabisulphite as an antioxidant (0.05% w/w)
Assessing up to three different wet fill weights, and two different matrix types
Assessing a minimum frozen hold time of 24 hours
Assessing the impact of solution hold time on the finished product

The mixes were to be dosed at the end of mixing (0 hours solution hold, SH0) and at approximately 24 hours after completion of the mixing stage (24 hours solution hold, SH24). The product had up to 24 hours frozen hold (FH) prior to freeze drying. The batches were to be freeze dried at a shelf temperature of 0° C. on an 18 hour cycle. Appearance and dispersion testing were to be performed on all batches.

The following Table 3 is the formulations used in the Solution-Based Formulation Study 2:

TABLE 3

| | | | | | | Sodium | pH (citric |
|---|---|---|---|---|---|---|---|
| | Gelatin | Gelatin | Mannitol | Epinephrine | Unit wet | metabisulphite | acid for |
| Batch | Type | % w/w | % w/w | % w/w | fill (mg) | % w/w | adjustment) |
| 1 | Fish | 5.00 | 4.00 | 4.00 | 500 | 0.05 | qs API |
| | (HMW) | | | | | | dissolution~pH 5.0 |
| 2 | Fish | 5.00 | 4.00 | 2.67 | 750 | | |
| | (HMW) | | | | | | |
| 3 | Fish | 5.00 | 4.00 | 2.00 | 1000 | | |
| | (HMW) | | | | | | |
| 4 | Bovine | 4.00 | 3.00 | 4.00 | 500 | | |
| | (Non-Ox) | | | | | | |
| 5 | Bovine | 4.00 | 3.00 | 2.67 | 750 | | |
| | (Non-Ox) | | | | | | |
| 6 | Bovine | 4.00 | 3.00 | 2.00 | 1000 | | |
| | (Non-Ox) | | | | | | |

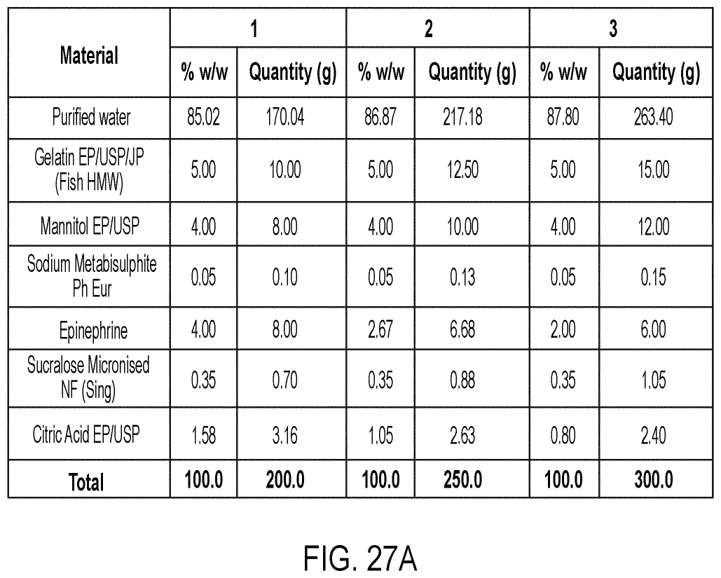

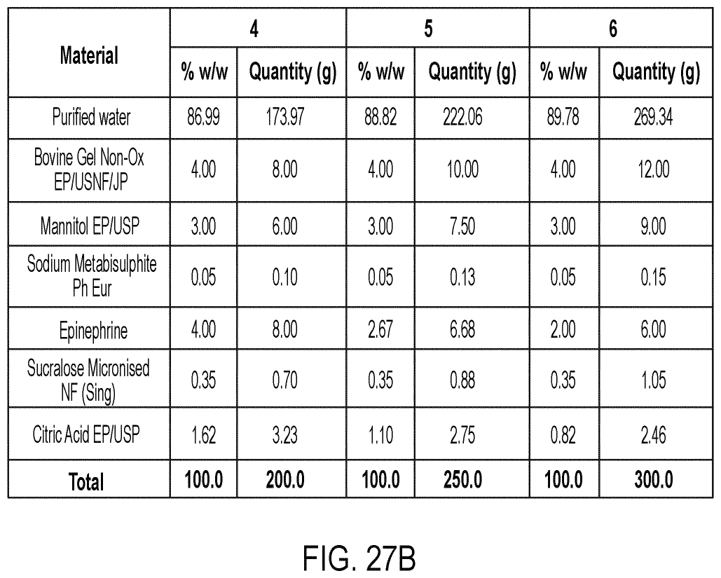

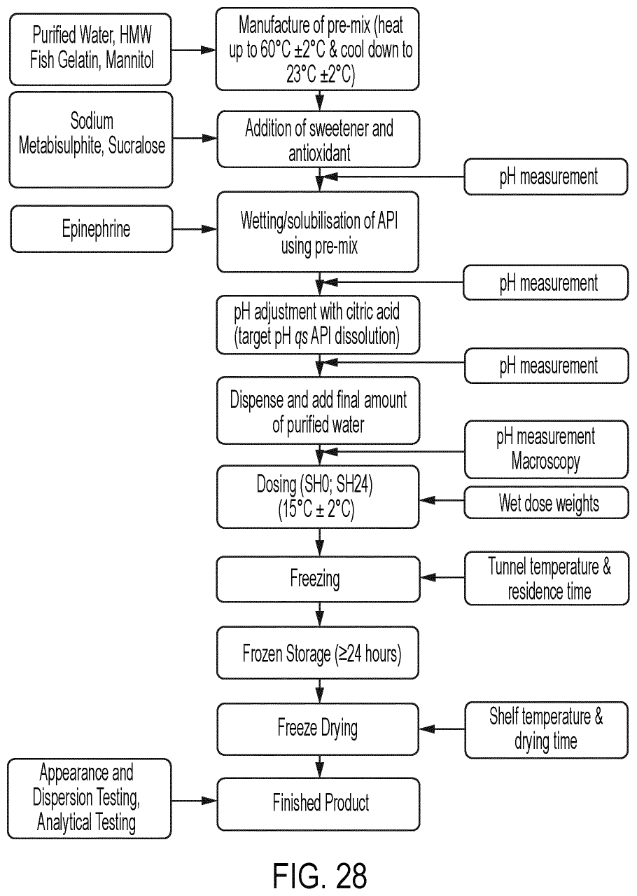

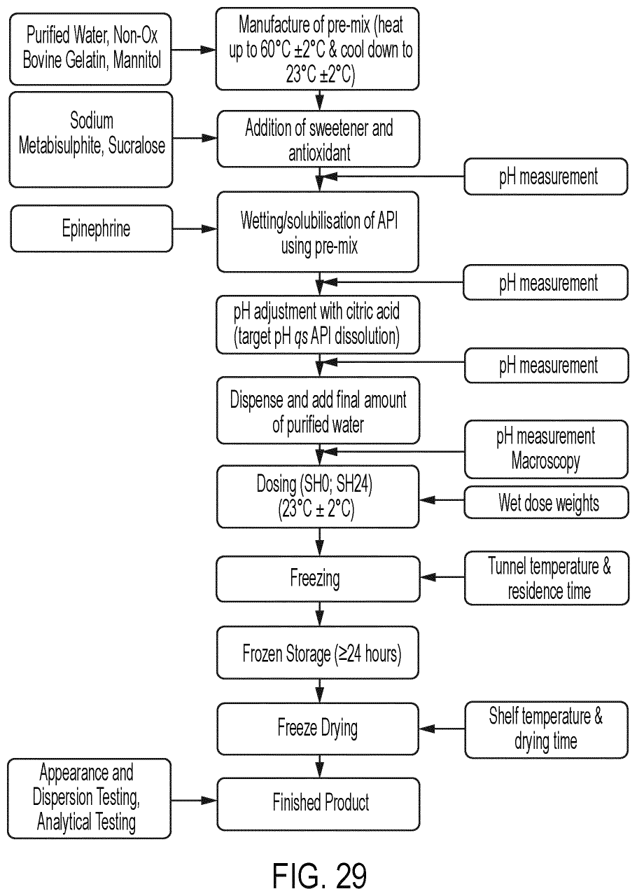

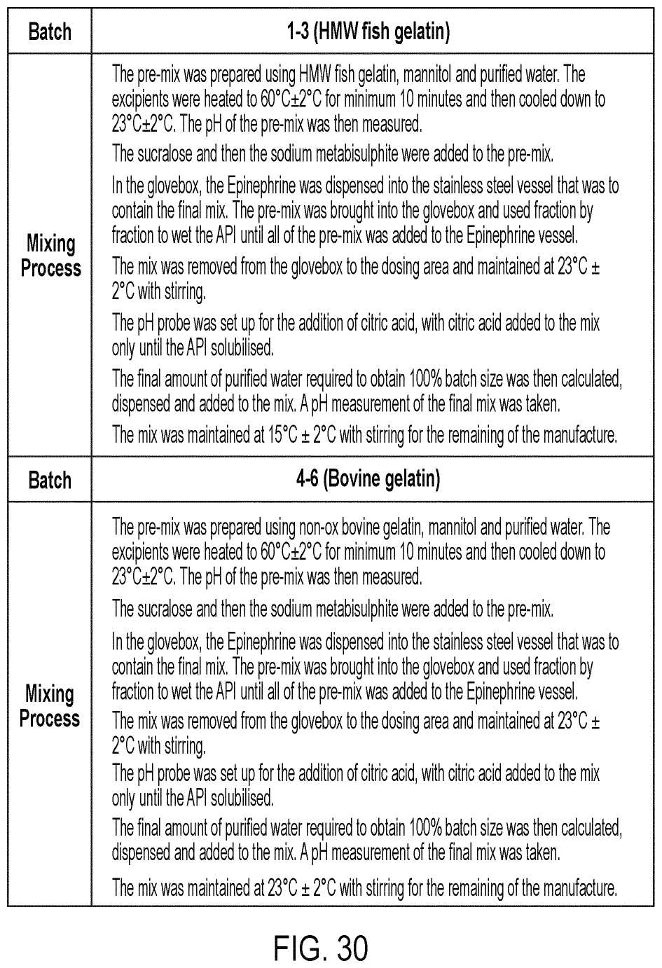

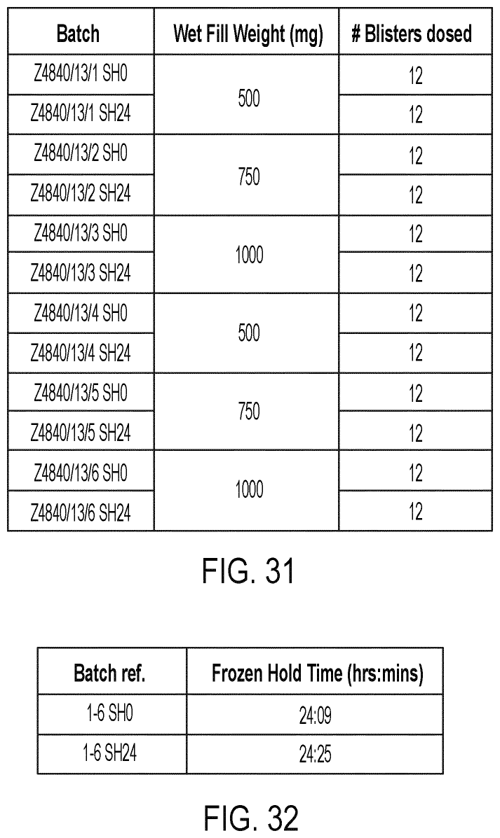

A more detail breakdown of the formulations used in Study 2 can be found in FIGS. 27A-B. The flowchart for creating the dosage forms in Solution-Based Formulation Study 2 can be found in FIGS. 28-29. The preparation of the mixes for Solution-Based Formulation Study was conducted as described in FIG. 30. FIG. 31 provides a table summarizing the dosing points, wet fill weights of the batches, and the number of blisters dosed per batch. The trays were frozen at −90° C. with a freeze tunnel residence time of 3:15 min. The time period from when the frozen product was transferred from the freeze tunnel to a freezer and stored below −15° C. (freezer set up at approximately −25° C.) until loading into the freeze dryer is referred to as the frozen hold (FH) time. Frozen hold can be a critical part of the process as this can allow for any amorphous mannitol to crystallize giving added strength to the tablet. The batches had a minimum frozen hold of 24 hours, with the range of frozen holding times for this Study 2 detailed in FIG. 32. Batches were dried at 0° C. for 18 hours. Two dryer loads were required as there were two dosing time-points.

All batches were sealed under 5 bar pressure at 185° C. with a dwell time of 1 second. For some units, the frost heaves were seen to be sticking to the lidding foil when the sealed blister was opened for inspection. This is consistent with the inspection data for this study where a proportion of units were observed to have a frost heave above the surface of the pack. While all blisters tested passed a vacuum leak test (VLT) to demonstrate seal integrity, contact with the lidding foil during sealing can cause damage to units.

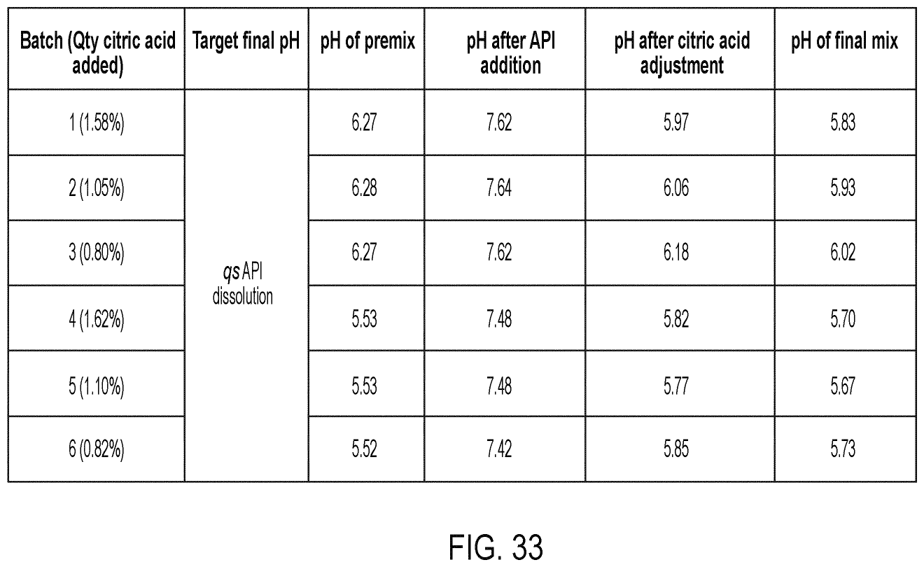

Citric acid was used to adjust the pH of the mix only until the API dissolved; this was anticipated to be approximately pH 5.0-5.2 based on observations from Study 1 above. The pH measurements that were taken for each batch at different stages of mix preparation are detailed in FIG. 33. A noticeable increase in pH was observed following the addition of the API to the mixes. As the pH was only to be adjusted to the point at which the API solubilized, a much smaller quantity of citric acid was used in these batches than in Study 1. A small reduction in pH was seen across all six batches between adjustment with citric acid and addition of water to obtain 100% batch size; this was not considered to be of any practical significance.

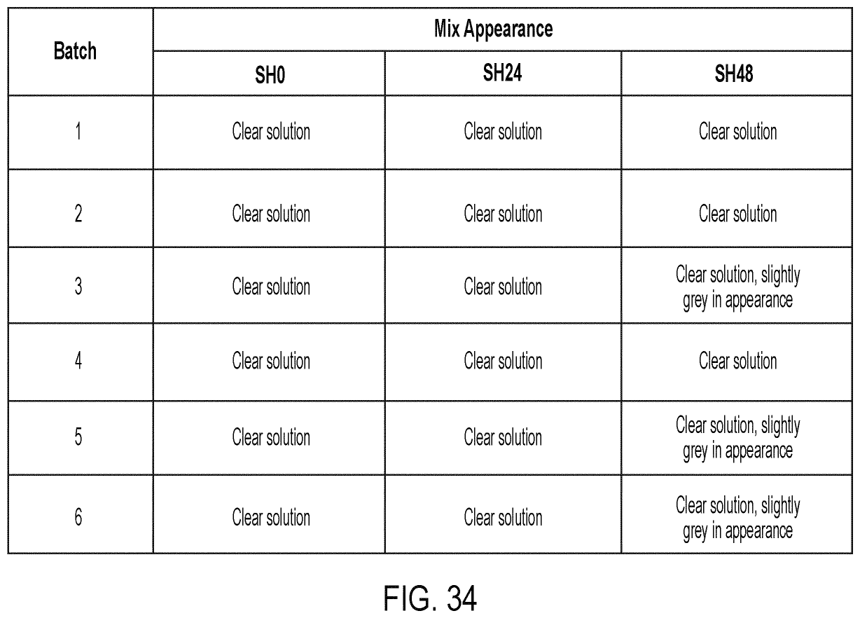

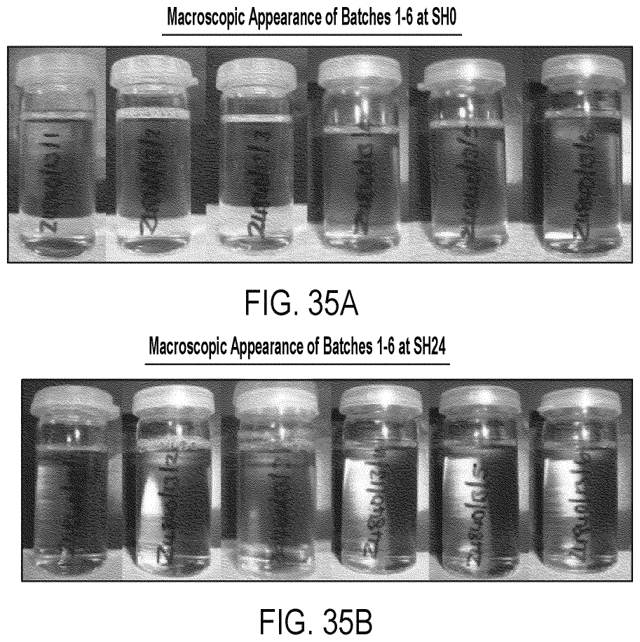

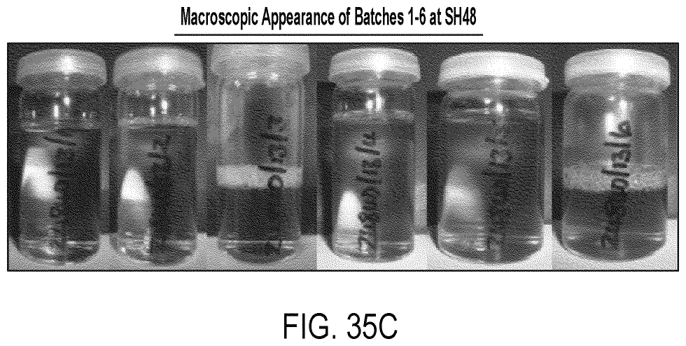

The appearance of the solutions was recorded at the end of mixing (SH0), after approximately 24 hours stirring (SH24) and after approximately 48 hours stirring (SH48). The observations recorded for each mix are summarized in FIG. 34 and illustrated in FIGS. 35A-C. There is no evident color change over time across all batches, which indicates that the sodium metabisulphite is successful as an antioxidant across all six formulations. This is a marked improvement on Study 1, where the mixes without sodium metabisulphite were discolored, indicating oxidation of Epinephrine. The macroscopic appearance of all six mixes appeared to stay consistent over the 48 hour mix hold, although batches 3, 5, and 6 were noted to be slightly grey in appearance after 48 hours solution hold.

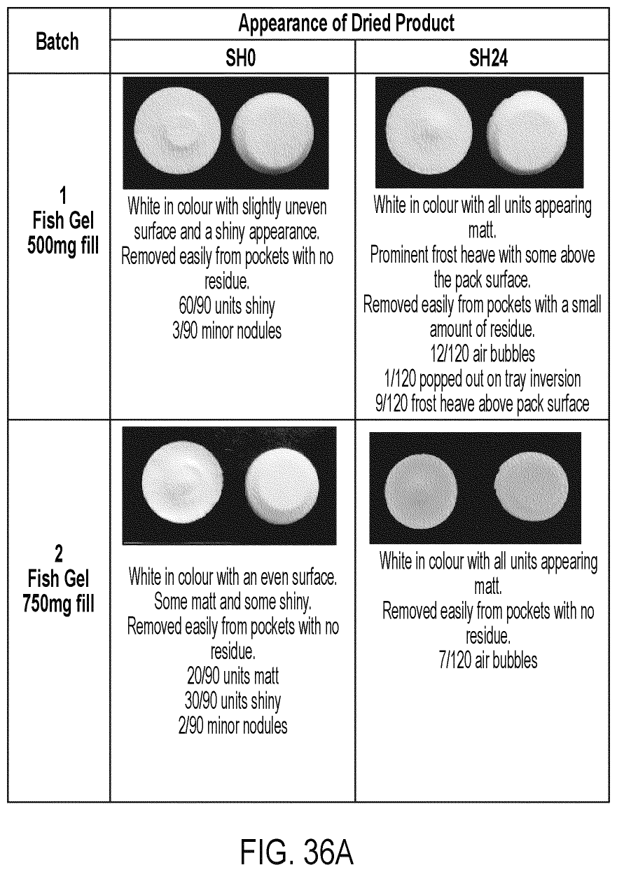

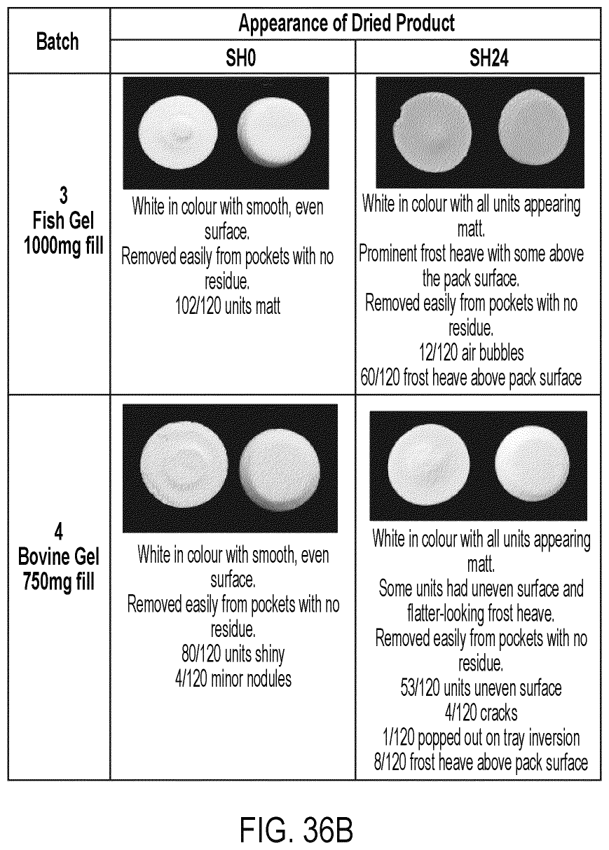

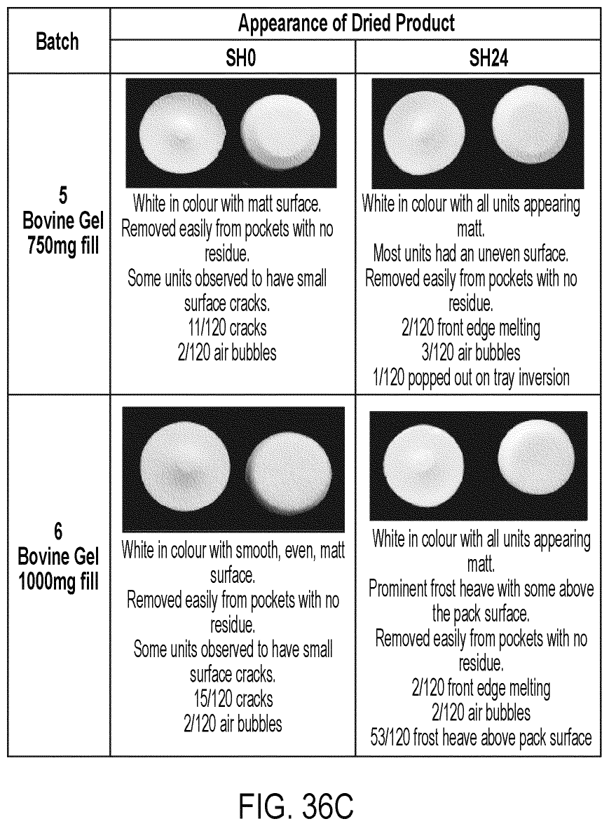

All batches were inspected and the appearance was considered good if the units were circular, freeze-dried tablets that could be removed from packaging without breaking and were generally free of defects. These observations of the freeze-dried tablets are in FIGS. 36A-C. Overall unit appearance for all batches was acceptable and better than Study 1, indicating that the high citric acid content is at least partially responsible for poor unit appearance seen in Study 1. The SH24 units across all batches appeared to have a matte surface, where some SH0 units from batches 1, 2, and 4 appeared to have a glossy surface. This suggests that longer solution hold may have had a beneficial effect on the appearance of the units. Overall units formulated with bovine gelatin appear to have a slightly better appearance compared to unit formulated with fish gelatin. A proportion of the frost heaves were above the pack surface for SH24 units of 500 mg and 1000 mg units.

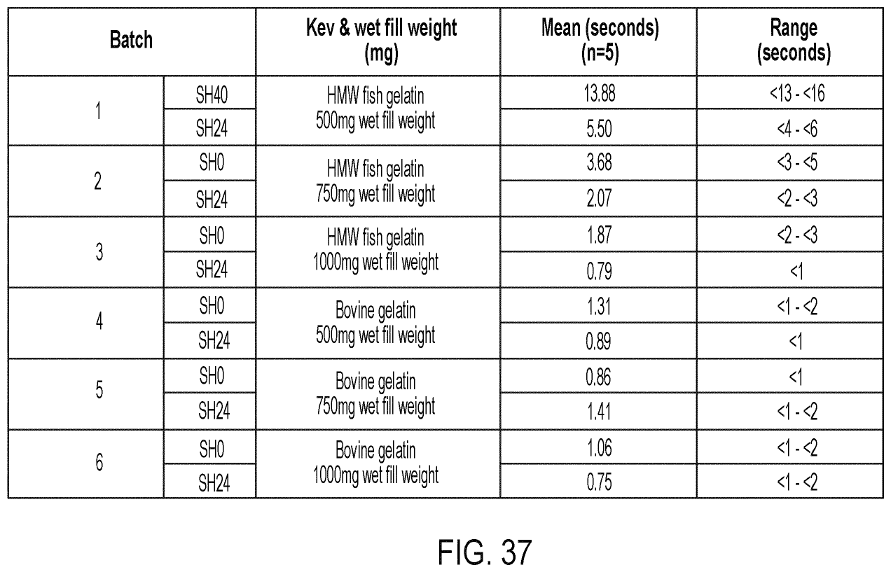

The dispersion time results for batches 1-6 are summarized in FIG. 37. With the exception of both sub-batches of batch 1, all batches dispersed in under 5 seconds, which is within the acceptable criterion of less than or equal to 10 seconds. Units from batch 1, for both dosing timepoints, dispersed notably more slowly than from all other batches, indicating a less suitable formulation for the product. In general, the units formulated with bovine gelatin appeared to disperse more quickly than those with fish gelatin.

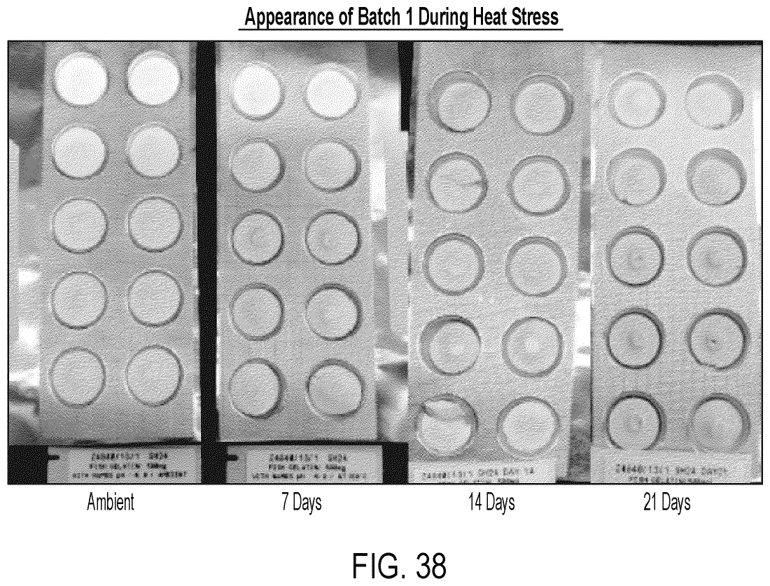

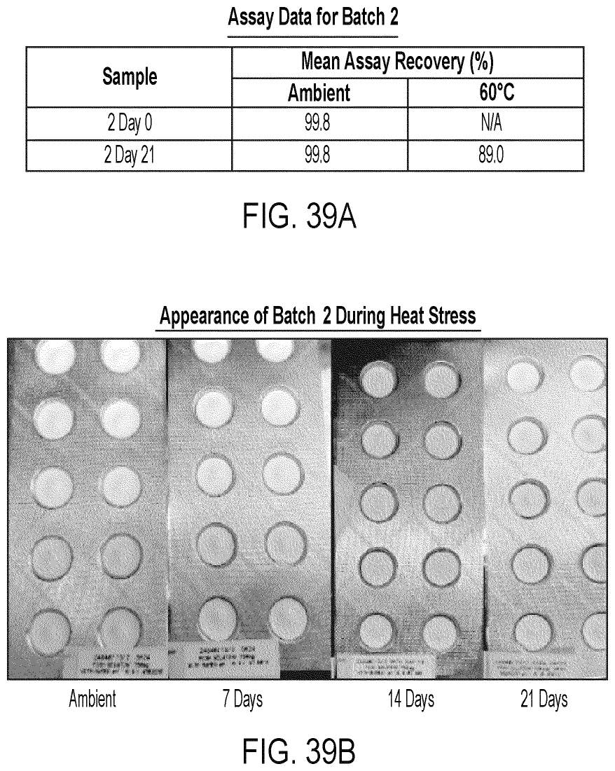

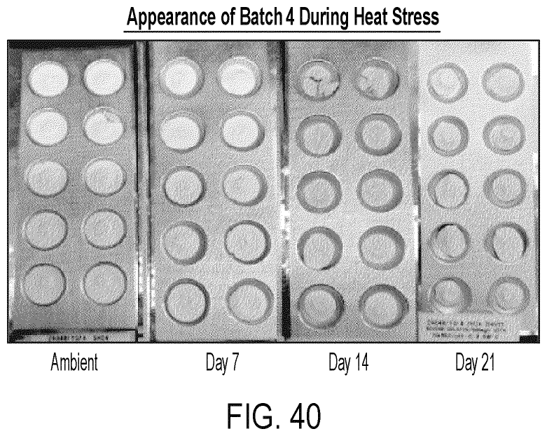

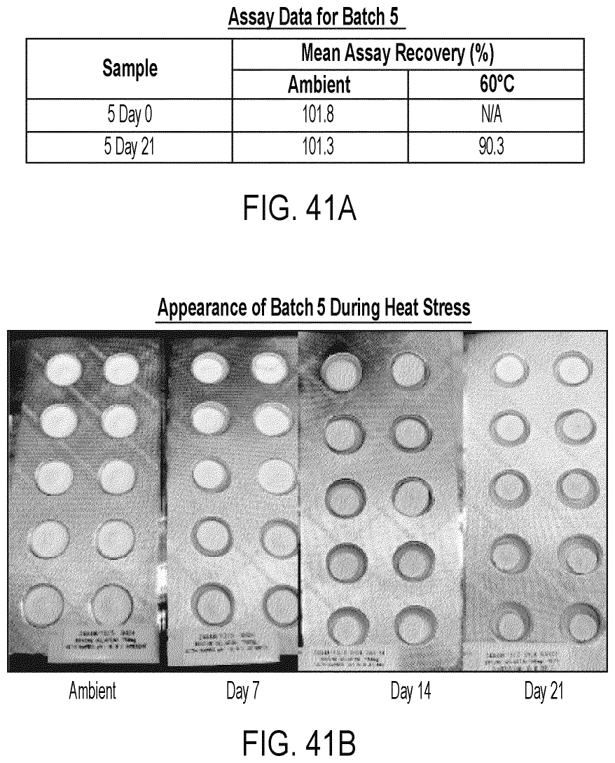

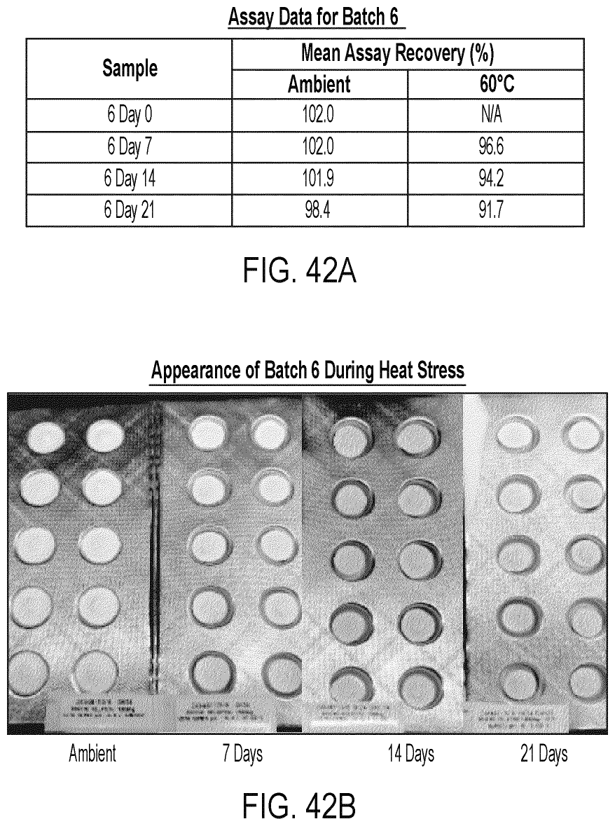

Sealed blisters from batches 1, 2, 4, 5, and 6 underwent a heat stress study, where the blisters were stored at 60° C. for up to 21 days in order to emulate longer-term stability testing conditions. Units were removed from the chamber every 7 days and assayed, as well as their appearance recorded. The results of these studies were to be used in order to influence future formulation factors such as proportion of antioxidant (i.e. sodium metabisulphite), or gelatin type. FIG. 38 shows the appearance of batch 1 during the heat stress study. The unit properties were acceptable at the initial time point, however slight color change, shrinkage, and fragility (i.e., cracking) are evident upon storage. An equivalent formulation at pH 3.5 Study 1, batch 1 was evaluated under heat stress. The appearance of the Study 2 units were marginally improved following heat stress than those from Study 1 however appearance of both sets of units following heat stress was still unacceptable. Results from batch 2 are presented in FIGS. 39A-B. Units from this batch were assayed at Day 0 of the heat stress study (i.e., ambient), and at Day 21. The ambient samples remain unchanged and within the 95-105% assay recovery limits after the 21 day study period but the assay recovery for the 60° C. samples falls significantly after the 21 day study period, suggesting this formulation is not well protected against oxidation and may not have a suitable shelf life. Visually the units from this batch had acceptable unit structure at the initial time-point, however there is slight color change and shrinkage evident over the course of the study, although not as much apparent shrinkage as other batches tested. Results from batch 4 are presented in FIG. 40. The unit properties were acceptable at the initial time-point, however more significant color change, shrinkage and fragility (i.e., cracking) was evident upon storage. An equivalent formulation at pH 3.5 was manufactured in Study 1, batch 3. The ambient condition for the Study 1 batch was assayed and resulted in 102.5% assay recovery however the unit appearance was unacceptable even at the initial time-point and shrinkage and color change were evident. Although the degree of color change in Study 2, batch 4 was smaller than the Study 1 batch, unit appearance from both the Study 1 batch and the Study 2 batch was unacceptable following heat stress. Results from batch 5 are presented in FIGS. 41A-B. The units from this batch were assayed at Day 0 (i.e., ambient) and Day 21. The ambient samples remain within the 95-105% assay recovery limits after the 21 day study period but the assay recovery for the 60° C. samples falls after the 21 day study period, suggesting this formulation is not well protected against oxidation and may not have a suitable shelf life. Visually the units from this batch had acceptable unit structure at the initial time-point, however there is notable color change, shrinkage and fragility (i.e., cracking) evident on storage. The units from this batch (made with bovine gelatin) appear to have shrunk slightly more than the equivalent formulation made with fish gelatin (batch 2). Results from batch 6 are presented in FIGS. 42A-B. The ambient samples remain within the 95-105% assay recovery limits over the 21 day study period but the assay recovery for the 60° C. samples falls over the 21 day study period, although not as sharply as the other batches assayed. Visually the units from this batch had acceptable appearance at the initial time-point, however there is slight color change and shrinkage evident on storage.

Overall, the incorporation of sodium metabisulphite into all batches appeared beneficial in reducing the degree of color change compared with Study 1 samples. In addition, fish gelatin appeared to offer an advantage in terms of less shrinkage compared to bovine gelatin.

Solution-Based Formulation Study 3

The objective of this study was to further evaluate the technical feasibility of developing an Epinephrine formulation that competes with the current EpiPen product. Units were manufactured both for use in a rabbit PK study for the purposes of dose ranging, and to be set down on stability.

The incorporate of the API into one matrix (HMW fish gelatin) was assessed. Three different unit wet fill weights were used (175 mg, 350 mg and 700 mg) in order to provide dose-proportional active doses of 5 mg, 10 mg and 20 mg respectively. Critical physical attributes of the dried units were to be evaluated, including appearance and dispersion time. Citric acid was to be used as a pH modifier to assess the effect of pH on mix properties and finished units in terms of chemical stability.

The mixes were dosed at the end of mixing (0 hours solution hold, SH0). The product had a minimum of 24 hours frozen hold (FH) prior to freeze drying. The batches were freeze dried at a shelf temperature of 0° C. on an 18 hour cycle. Appearance and dispersion testing was performed on all batches.

The following Table 4 is the formulations used in the Solution-Based Formulation Study 3:

TABLE 4

| | | | Sodium | pH (citric | |
|---|---|---|---|---|---|
| | Sub- | Epinephrine | metabisulphite | acid for | Unit Wet Fill |
| Batch | batch | % w/w | % w/w | adjustment) | Weight (mg) |
| 1 | A | 2.86 | 0.30 | 6.0 ± 0.2 | 175 |
| | B | | | | 350 |
| | C | | | | 700 |
| 2 | A | 2.86 | 0.30 | 5.0 ± 0.2 | 175 |
| | B | | | | 350 |
| | C | | | | 700 |

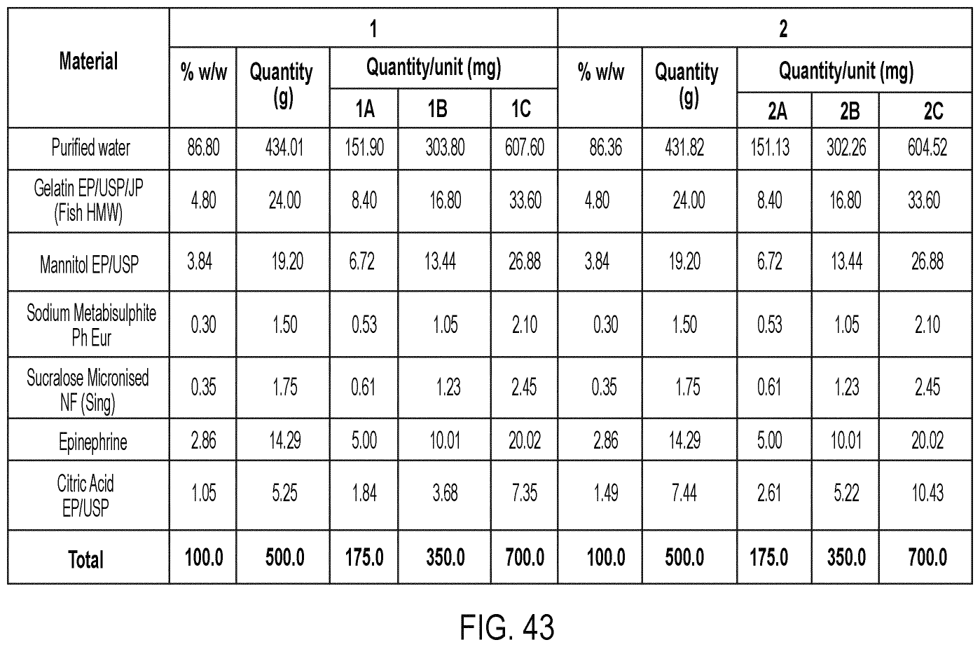

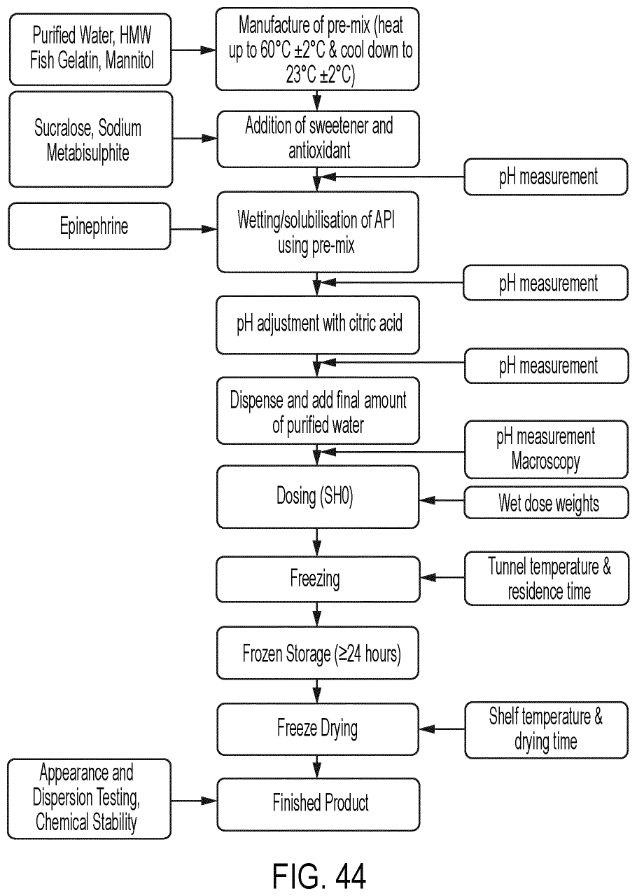

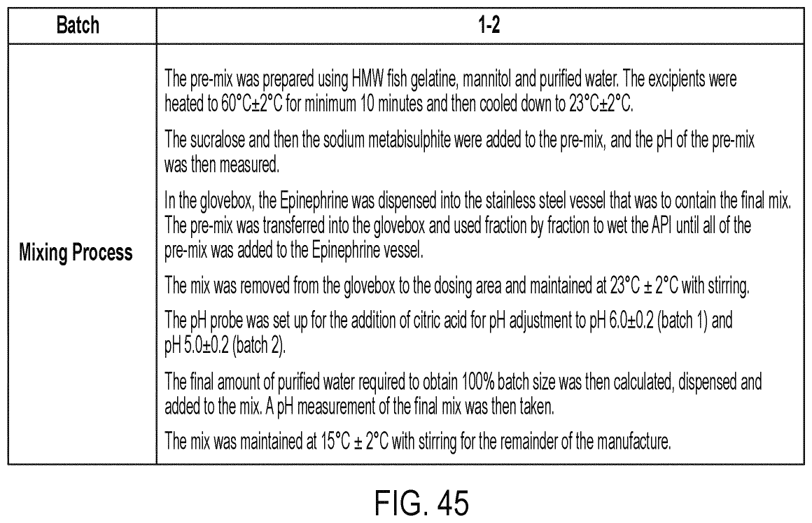

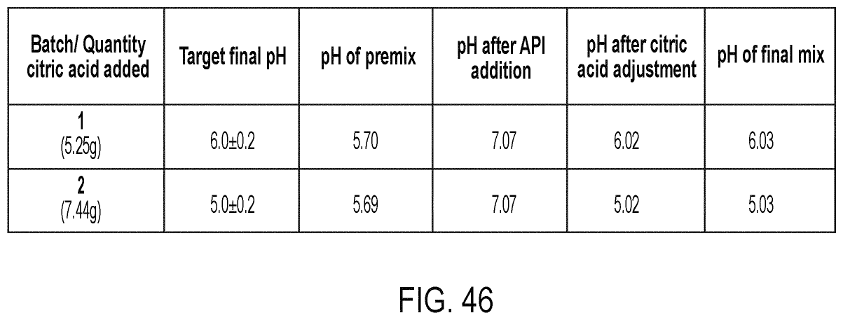

More details on the formulations of Batches 1-2 of this Study can be found in FIG. 43. FIG. 44 provides a flowchart for the manufacturing process of Batches 1-2. The preparation of the mixes for batches 1-2 can be found in FIG. 45. No problems were recorded during dosing although some units from batches 1A and 2A were seen to be wedge-shaped due to the pocket geometry and fill size used for these batches; both dosed a 175 mg wet fill into a 250 mg pocket. All batches were dosed immediately after the end of mixing (SH0). The trays were frozen at −90° C. with a freeze tunnel resident time of 3:15 min. The time period during which the frozen product is transferred from the freeze tunnel to a freezer and stored below −15° C. (freezer set up at approximately −25° C.) until loading into the freeze dryer is referred to as the frozen hold (FH) time. Frozen hold can be a critical part of the process for some products, as this allows for amorphous mannitol to crystalize giving added strength to the tablet. The batches had a minimum frozen hold of 24 hours. Batches were freeze-dried at 0° C. for 18 hours. All batches were sealed under 5 bar pressure at 185° C. with a dwell time of 1 second. All blisters tested passed the vacuum leak test with no observed shrinkage, indicating that sealing conditions employed were suitable for this product. Citric acid was used to adjust the pH of the mixes. The pH measurements that were taken for each batch at different stage of mix preparation are detailed in FIG. 46.

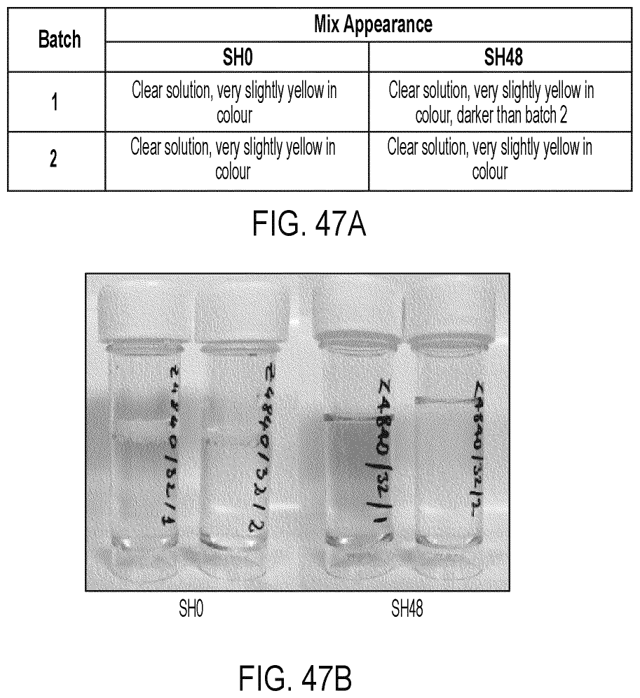

The appearance of the solutions were recorded at the end of mixing (SH0) and after approximately 48 hours sitting (SH48). The observations recorded for each mix are summarized in FIGS. 47A-B. There is no evident color change over time across batch 2, which indicates that the sodium metabisulphite is an effective antioxidant in this formulation at lower pH. However, the mix for batch 1 (formulated at higher pH) appeared to become slightly darker over the 48 hour mix hold. This indicates that some oxidation may have taken place in the higher pH formulation.

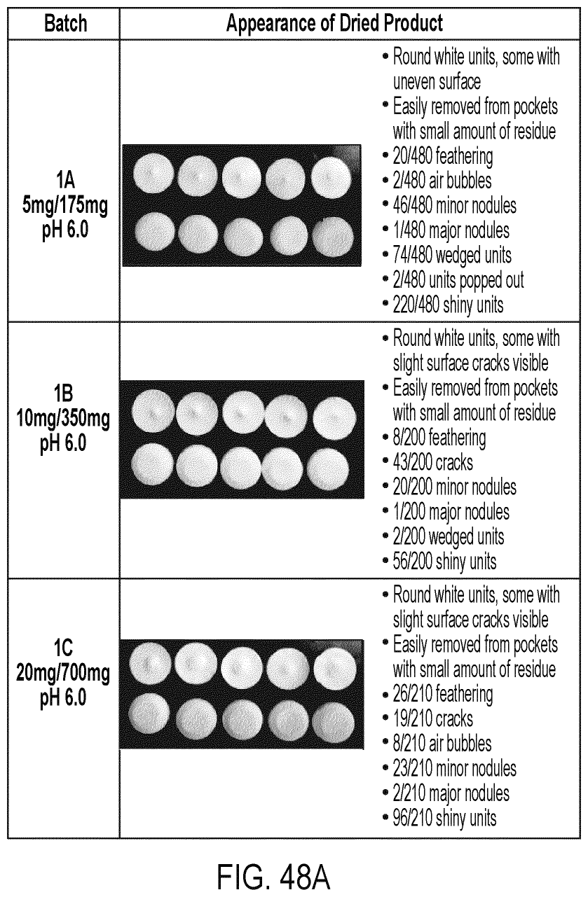

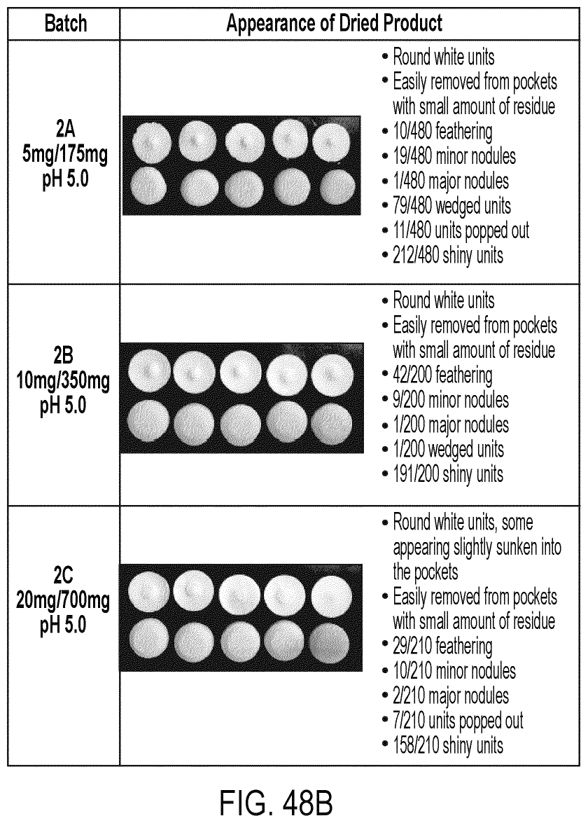

The appearance of all batches as considered good if the units were circular, freeze-dried tablets that could be removed from packaging without breaking and were generally free of defects. FIGS. 48A-B contain the appearance observation and noted defects of the batches in this Study 3. Overall unit appearance for all batches was acceptable for the purposes of this study; all batches producing round white units with some cosmetic defects noted. Feathering was observed across all six sub-batches; this is likely due to bench-scale related processing factors and would be expected to be eliminated at a larger manufacturing scale. Nodules were observed across all six sub-batches; this is a common observation when using fish gelatin and is likely to be the result of the bench-scale freezing process. Some small surface cracks were observed for batches 1B and 1C. This is potentially due to the proportions of matrix formers (i.e., gelatin and mannitol) within the formulation, or the pH level of the formulation 9 pH 6 for these batches). Due to pocket geometry that was required to be used in order to be able to seal the unit blisters, a notable proportion of minor wedge shaped units were seen for batches 1A and 2A in which a wet fill weight of 175 mg was dosed into a 250 mg pocket. A large proportion of the units from all sub-batches had a shiny appearance. The occurrence of the surface shine may be attributable to the relatively large proportion of citric acid required within the formulation to reach target pH.

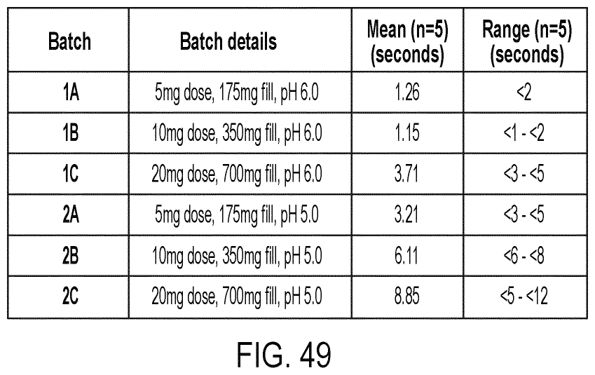

The dispersion time results for batches 1-2 are summarized in FIG. 49. All batches with the exception of batch 2C dispersed in under 8 seconds, which is within the acceptable limit of less than or equal to 10 seconds. Batches 1A-C, which were adjusted to the higher pH level, had much faster dispersion times than batches 2A-C, which were formulated at a lower pH. This indicates that the quantity of citric acid within the formulation may influence the finished product dispersion rate.

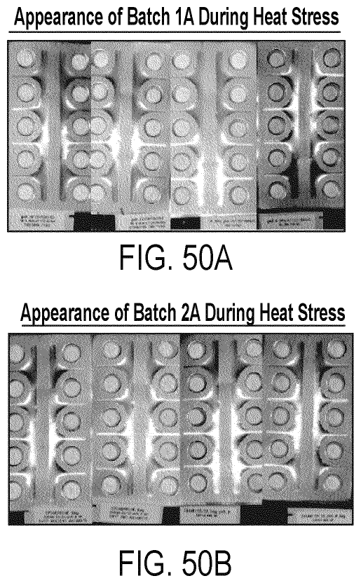

Sealed blisters of the 5 mg dosage strength from both batches 1A and 2A underwent a heat stress study; blisters were stored at 60° C. for up to 21 days to emulate longer-term stability testing conditions. Units were removed from the chamber every 7 days and assayed for Epinephrine content, related substances and Sodium metabisulphite content. The unit appearance was also recorded and shown in FIGS. 50A-B.

The following table 5 provides the analytic results obtained over 21 days for Batch 1A:

TABLE 5

| | Epinephrine | Sodium | Impurity F | Total Impurities |
|---|---|---|---|---|
| | (% Label | metabisulphite | (RRT~0.8) | (Unknown + |
| Sample | Claim) | (% Label Claim) | (%) | Impurity F) (%) |
| Batch 1A, Day 0 | 95.9 | 50.4 | 4.46 | 4.63 |
| Batch 1A, Day 7 | 84.5 | 4.8 | 20.28 | 22.78 |
| Batch 1A, Day 14 | 82.3 | None detectable | 19.80 | 24.93 |
| Batch 1A, Day 21 | 78.0 | 1.0 | 19.15 | 29.57 |

The data above shows a continuous decline in Epinephrine assay recovery throughout the 21 day study period. This falls outside the limit of 90-110% assay recovery label claim, which is based on the BP pharmacopeia specification for Adrenaline (Epinephrine) Injection, with a total percentage loss of 17.9%. The Sodium metabisulphite assay shows that the antioxidant is rapidly consumed; this correlates with the formation of ‘impurity F’, which is known to form following a reaction between the antioxidant and Epinephrine. Furthermore, the total impurities quickly accumulate to exceed the limit of 16% (see below) upon consumption of Sodium metabisulphite. The analytical data also shows that a significant proportion of the antioxidant is being consumed during the manufacturing process (% label claim at initial time point of 50.4%). These results indicate that this formulation is not well protected against oxidation and may not have a suitable shelf life.

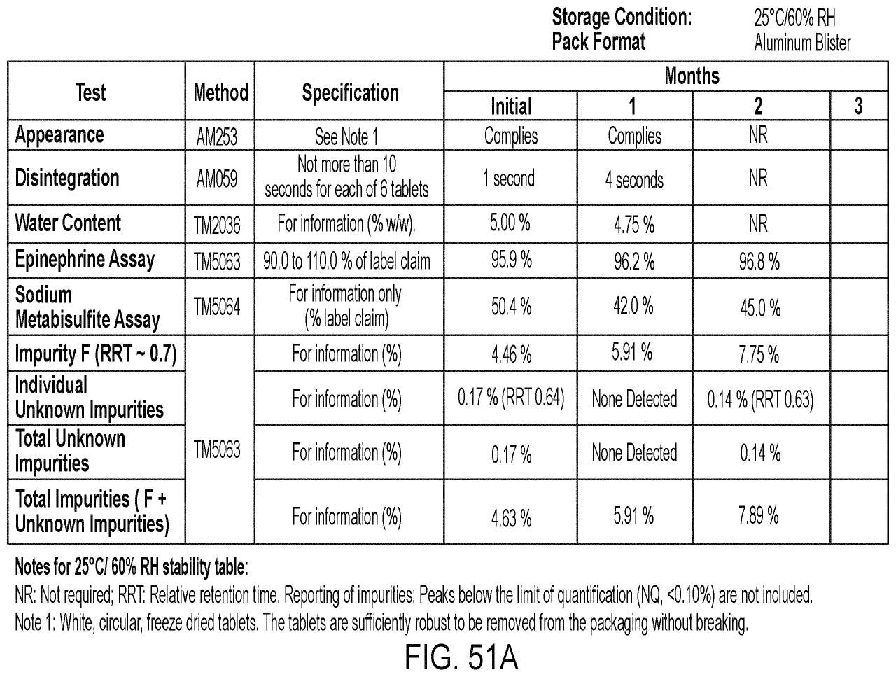

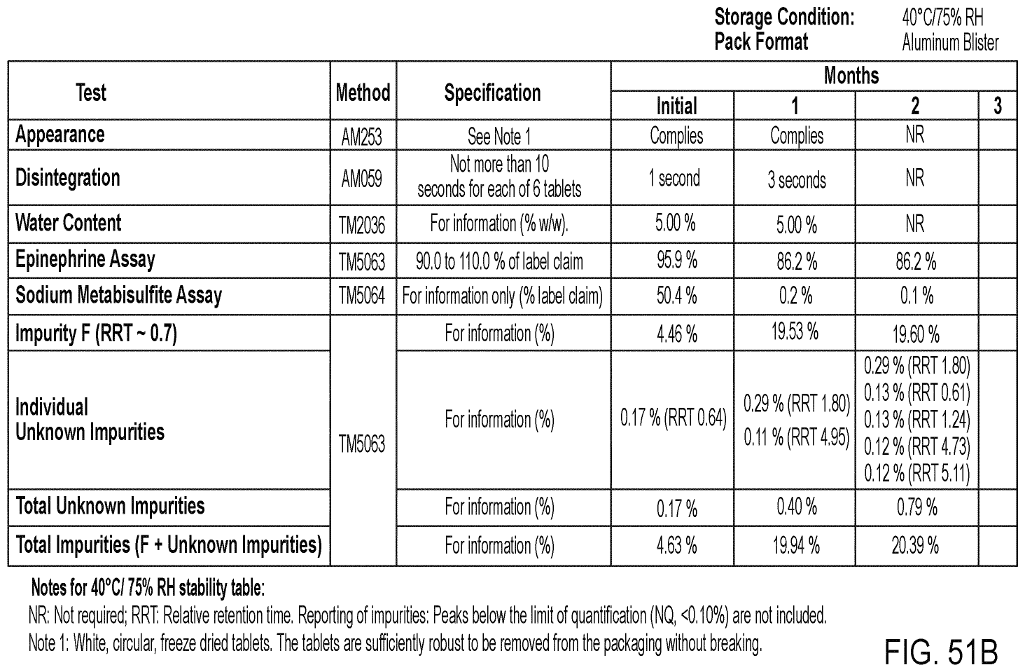

5 mg samples from Batch 1A were placed on store at 25° C./60% RH and 40° C./75% RH with testing to be performed at initial and after 1 month, 2 months, and 3 months. FIGS. 51A-B provide a summary of results from the testing of the initial, 1 month, and 2-month time point samples at 25° C./60% RH (FIG. 51A) and 40° C./75% RH (FIG. 51B). The appearance of the tablets was assessed at initial and after 1-month storage at 25° C./60% RH and 40° C./75% RH. The appearance complied with specification at all time points and stability conditions. The unit properties of Batch 1A were acceptable at the initial time point, but significant color change and slight shrinkage of the units was evident upon storage. The appearance of the tablets was not assessed at the 2-month time point.

The disintegration of the tablets was assessed at initial and after 1-month storage at 25° C./60% RH and 40° C./75% RH according to the pharmacopoeal disintegration test (USP 701). Results complied with the specification of not more than 10 seconds. The disintegration of the tablets was not assessed at the 2-month time point. The samples were also tested for water content according to coulometric karl-fischer titration. For the 1-month stability samples, moisture content results of 4.75% w/w for the 25° C./60% RH samples and 5.00% w/w for the 40° C./75% RH were obtained. There is a slight decrease of 0.25 w/w for the 25° C./60% RH samples from the initial testing moisture result of 5.00 w/w. The result from 40° C./75% RH samples has remained unchanged at the 1-month time point from the initial result. The water content was not assessed at the 2-month time point.

Batch 1A samples were also assay tested. For the 25° C./60% RH condition, results of 96.2% and 96.8% were generated at the 1-month and 2-month time point respectively. These represent a slight increase of 0.3% and 0.9% from the initial result of 95.9% which was generated at the initial time point. These results are considered within experimental variation for the methodology. For the 40° C./75% RH condition, a result of 86.2% was generated at both the 1-month and 2-month time point. This represents a significant decrease of 12.4% form the initial result of 95.9% which was generated at the initial time point and is below the specification of 90-110% label claim, which is based on the BP pharmacopeia specification for Adrenaline (Epinephrine) Injection.

Sodium metabisulfite assay testing was also conducted on the samples using HPLC. At initial, a mean result of 50.4% was obtained. For the 25° C./60% RH condition, this decreased to 42% and 45% at the 1-month and 2-month time point. For the 40° C./75% RH condition, this decreased to 0.2% and 0.1% at the 1-month and 2-month time point and showed that all the antioxidant had been consumed in this formulation.

The assessment of degradation products was performed according to HPLC. Results generated were assessed with reference to the limits outlined in the BP monograph for Adrenaline Injection and are as follows:
D-Adrenaline: Not more than 15% (D-Adrenaline content is assessed on a chiral HPLC method. This was not performed on samples from this study and so has not been included in this assessment.)
Impurity F (Epinephrine Sulfonic acid): Not more than 15%
Impurity B (Norepinephrine): Not more than 1% (Impurity B was found to co-elute with Impurity F on this draft method, so it could not be assessed individually.)
Unknown Individual Impurity: Not more than 0.5%
Total of Unknown Individual Impurities: Not more than 1%
Total of all impurities including D-Adrenaline: Not more than 16%

For the 25° C./60% RH condition, the levels of impurity F were 4.4% at initial, increasing to 5.91% at 1 month and 7.75% at 2 months. Total unknown impurities were at 0.17% at initial. At the 2-month time point this was 0.14%. Total impurities (impurity F and unknown impurities) were 4.63% at initial, increasing to 5.91% at 1 month and 7.89% at 2 months. All results remain within the limits listed in the BP monograph for Adrenaline Injection.

For the 40° C./75% RH condition, the levels of impurity F were 4.46% at initial, increasing to 19.53% at 1 month and 19.6% at 2 months. Total unknown impurities were at 0.17% at initial. At 1 month this increased to 0.40% and at 2 months this was increased to 0.79%. Total impurities (impurity F and unknown impurities) were 4.63% at initial, increasing to 19.94% at 1 month and 20.39% at 2 months. The levels of impurity F and the total impurities in this product after 1 month storage at 40° C./75% RH were above the limits listed in the BP monograph for Adrenaline Injection. As such, the 5 mg batch 1A failed assay specification after 1 month storage at 40° C./75% RH with a result below 90% label claim. In addition, the levels of impurity F and total impurities were above the limits listed in the BP monograph for Adrenaline Injection after 1 month storage at 40° C./75% RH. All results from the 25° C./60% RH storage condition met specification at the 2-month time point.

The following table 6 provides the analytic results obtained over 21 days for Batch 2A:

TABLE 6

| | Epinephrine | Sodium | Impurity F | Total Impurities |
|---|---|---|---|---|
| | (% Label | metabisulphite | (RRT~0.8) | (Unknown + |
| Sample | Claim) | (% Label Claim) | (%) | Impurity F) (%) |
| Batch 2A, Day 0 | 100.0 | 19.7 | 1.51 | 1.63 |
| Batch 2A, Day 7 | 94.6 | 1.4 | 7.93 | 9.24 |
| Batch 2A, Day 14 | 88.1 | 0.1 | 8.07 | 16.57 |
| Batch 2A, Day 21 | 83.3 | 0.1 | 9.05 | 18.26 |

The unit properties for Batch 2A were acceptable at the initial time point, however a significant color change and slight shrinkage of the units is evident upon storage. Analytical data shows a continuous decline in Epinephrine assay recovery throughout the 21 day study period. This falls outside the limit of 90-110%³ assay recovery, with a total percentage loss of 16.7%. The Sodium metabisulphite assay shows that the antioxidant is rapidly consumed; this correlates with the formation of impurity F, which is known to form following a reaction between the antioxidant and Epinephrine. Furthermore, the total impurities quickly accumulate to exceed the limit of 16% upon consumption of Sodium metabisulphite. The analytical data also shows that a significant proportion of the antioxidant is being consumed during the manufacturing process (% label claim at initial time point of 19.7%). These results indicate that this formulation is not well protected against oxidation and may not have a suitable shelf life, although suggest superior stability when compared to the units formulated at a higher pH (Batch 1A). The data suggests that the antioxidant may function better, or that Epinephrine is more stable at lower pH conditions. However, once the sodium metabisulphite has been depleted, degradation products other than impurity F are more rapidly formed with the lower pH formulation.

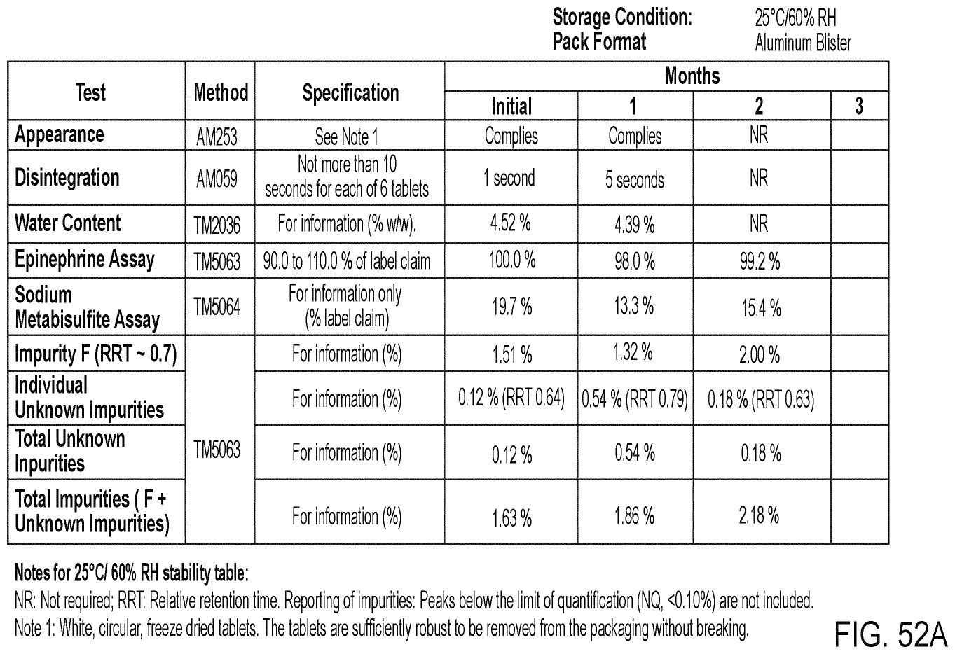

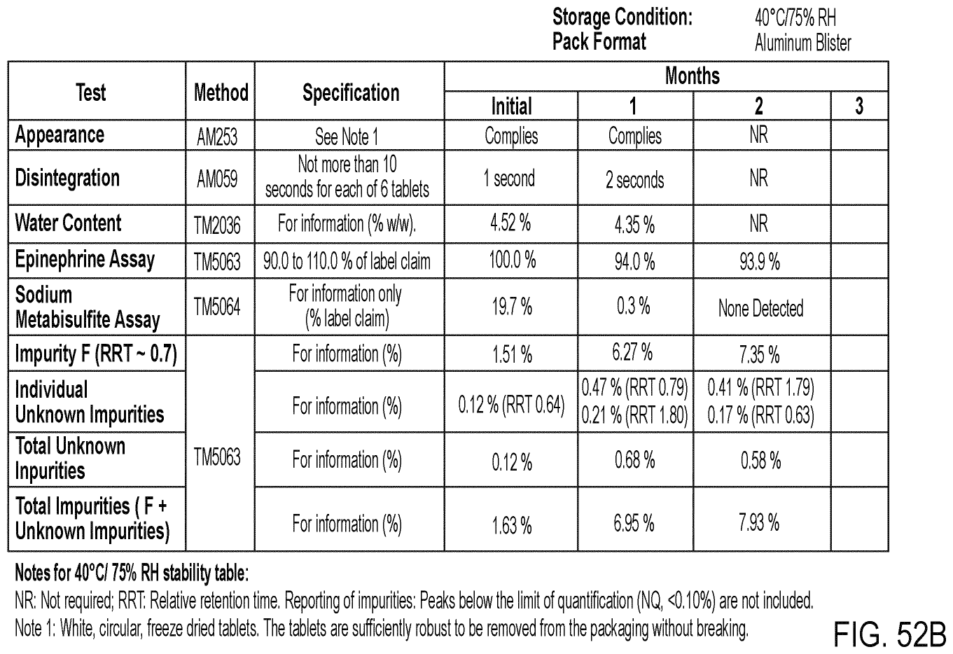

5 mg samples from Batch 2A were placed on store at 25° C./60% RH and 40° C./75% RH with testing to be performed at initial and after 1 month, 2 months, and 3 months. FIGS. 52A-B provide a summary of results from the testing of the initial, 1 month, and 2-month time point samples at 25° C./60% RH (FIG. 52A) and 40° C./75% RH (FIG. 52B). The appearance of the tablets was assessed at initial and after 1-month storage at 25° C./60% RH and 40° C./75% RH. The appearance complied with specification at all time points and stability conditions. The appearance of the tablets was not assessed at the 2-month time point.

The disintegration of the tablets was assessed at initial and after 1-month storage at 25° C./60% RH and 40° C./75% RH according to the pharmacopoeal disintegration test (USP 701). Results complied with the specification of not more than 10 seconds. The disintegration of the tablets was not assessed at the 2-month time point. The samples were also tested for water content according to coulometric karl-fischer titration. For the 1-month stability samples, moisture content results of 4.39% w/w for the 25° C./60% RH samples and 4.35% w/w for the 40° C./75% RH were obtained. There is a slight decrease of 0.13% w/w and 0.17% w/w respectively from the initial testing moisture result of 4.52% w/w. The water content was not assessed at the 2-month time point.

Batch 2A samples were also assay tested. For the 25° C./60% RH condition, results of 98% and 99.2% were generated at the 1-month and 2-month time point respectively. These represent a slight increase of 2.0% and 0.8% from the initial result of 100% which was generated at the initial time point. These results are considered within experimental variation for the methodology. For the 40° C./75% RH condition, results of 94% and 93.9% were generated at the 1-month and 2-month time point respectively. This represents a significant decrease of 6% to 6.1% from the initial result of 100% which was generated at the initial time point.

Sodium metabisulfite assay testing was also conducted on the samples using HPLC. At initial, a mean result of 19.7% was obtained. For the 25° C./60% RH condition, this decreased to 13.3% and 15.4% at the 1-month and 2-month time point. For the 40° C./75% RH condition, this decreased to 0.3% and 0% (none detected) at the 1-month and 2-month time point and showed that all the antioxidant had been consumed in this formulation.

The assessment of degradation products was performed according to HPLC. Results generated were assessed with reference to the limits outlined in the BP monograph for Adrenaline Injection and are as follows:
D-Adrenaline: Not more than 15% (D-Adrenaline content is assessed on a chiral HPLC method. This was not performed on samples from this study and so has not been included in this assessment.)
Impurity F (Epinephrine Sulfonic acid): Not more than 15%
Impurity B (Norepinephrine): Not more than 1% (Impurity B was found to co-elute with Impurity F on this draft method, so it could not be assessed individually.)
Unknown Individual Impurity: Not more than 0.5%
Total of Unknown Individual Impurities: Not more than 1%
Total of all impurities including D-Adrenaline: Not more than 16%

For the 25° C./60% RH condition, the levels of impurity F were 1.51% at initial, decreasing to 1.32% at 1 month and increasing to 2% at 2 months. Total unknown impurities were at 0.12% at initial. At the 1-month time point this increased to 0.54% before decreasing to 0.18%. Total impurities (impurity F and unknown impurities) were 1.63% at initial, increasing to 1.86% at 1 month and 2.18% at 2 months. All results remain within the limits listed in the BP monograph for Adrenaline Injection.

For the 40° C./75% RH condition, the levels of impurity F were 1.51% at initial, increasing to 6.27% at 1 month and 7.35% at 2 months. Total unknown impurities were at 0.12% at initial. At 1 month this increased to 0.68% and at 2 months this was recorded at 0.58%. Total impurities (impurity F and unknown impurities) were 1.63% at initial, increasing to 6.95% at 1 month and 7.93% at 2 months. All results remain within the limits listed in the BP monograph for Adrenaline Injection, although an individual unknown impurity at an RRT of 1.8 has increased from 0.21% at 1 month to 0.41% at 2 months, which is close to the limit of 0.5% for an individual unknown impurity. As such, the 5 mg batch 2A appeared to comply with specification after 2-month storage at 25° C./60% RH and 40° C./75% RH. However, assay results have shown a significant decrease of over 6% label claim after 2-month storage at 40° C./75% RH. In addition, the levels of the individual unknown impurity (RRT 1.8) have increased to 0.41% after 2-month storage at 40° C./75% RH. This is close to the limit of 0.5% for an individual unknown impurity listed in the BP monograph for Adrenaline Injection.

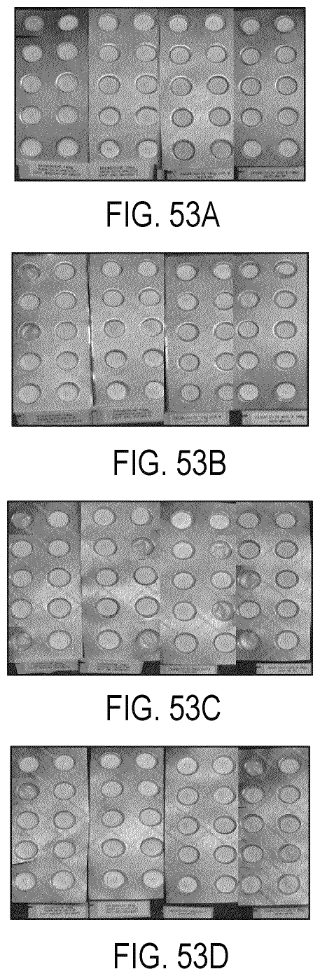

FIGS. 53A-D include the appearance of batches 1B, 2B, 1C, and 2C, respectively. All batches were acceptable at the initial time point, however a significant color change and sight shrinkage of the units is evident upon storage.

Solution-Based Formulation Optimization Study

The objective of this study was to optimize the solution-based Epinephrine formulation (as identified during the feasibility phases above), ensuring that the target product profile and critical quality attributes are met and that competes with the current EpiPen® product is achieved. Based on knowledge regarding antioxidant consumption during manufacture, centre-point formulations and those with highest antioxidant levels were to be set down on stability.

The incorporation of the API into one matrix (HMW fish gelatin) was to be assessed. A unit wet fill weight of 700 mg was used, providing a dosage strength of 20 mg for each batch (2.86% w/w Epinephrine). Critical physical attributes of the dried units were to be evaluated, including appearance and dispersion time. Citric acid was to be used as a pH modifier to assess the effect of pH on mix properties and finished units in terms of chemical stability.

The mixes were to be dosed at the end of mixing (0 hours solution hold, SH0), and after 24 hours of solution hold (SH2). The product had a minimum of 24 hours frozen hold (FH) prior to freeze drying. The batches were freeze dried at a shelf temperature of 0° C. on an 18 hour cycle. Appearance and dispersion testing was performed on all batches.

The following Table 7 includes the formulations for this optimization study:

TABLE 7

| | | | | Sodium | pH |
|---|---|---|---|---|---|
| | Sub- | Gelatin | Glycine | metabisulfite | (citric acid |
| Batch | batch | (% w/w) | (% w/w) | (% w/w) | for adjustment) |
| (Cycle 1) | 1 | 4.50 | 1.00 | 0.15 | 4.8 ± 0.1 |
| | 2 | 4.50 | 0.00 | 0.016 | 4.8 ± 0.1 |
| | 3 | 4.50 | 0.00 | 0.15 | 6.0 ± 0.1 |
| | 4 | 4.80 | 0.50 | 0.05 | 5.4 ± 0.1 |
| | 5 | 5.10 | 0.00 | 0.15 | 4.8 ± 0.1 |
| | 6 | 4.50 | 1.00 | 0.15 | 6.0 ± 0.1 |
| (Cycle 2) | 1 | 5.10 | 1.00 | 0.016 | 4.8 ± 0.1 |
| | 2 | 5.10 | 1.00 | 0.15 | 6.0 ± 0.1 |
| | 3 | 5.10 | 0.00 | 0.016 | 6.0 ± 0.1 |
| | 4 | 4.50 | 1.00 | 0.016 | 6.0 ± 0.1 |
| | 5 | 4.80 | 0.50 | 0.05 | 5.4 ± 0.1 |
| | 6 | 4.50 | 1.00 | 0.016 | 4.8 ± 0.1 |

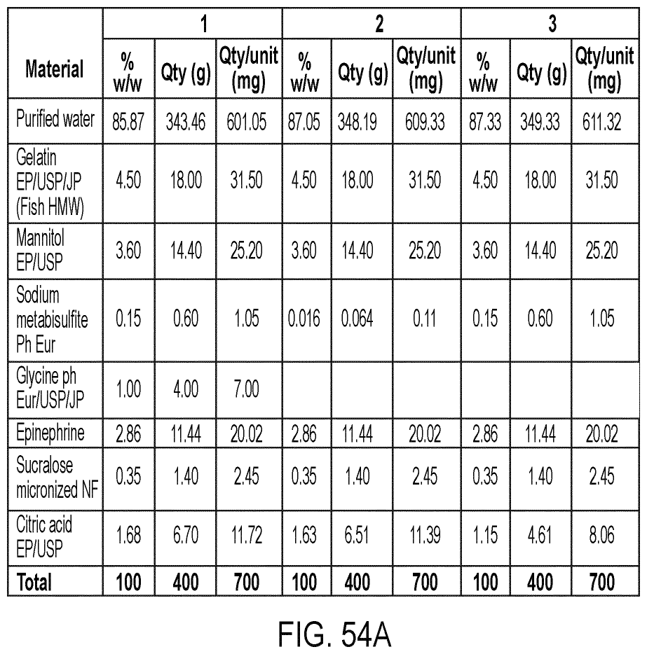

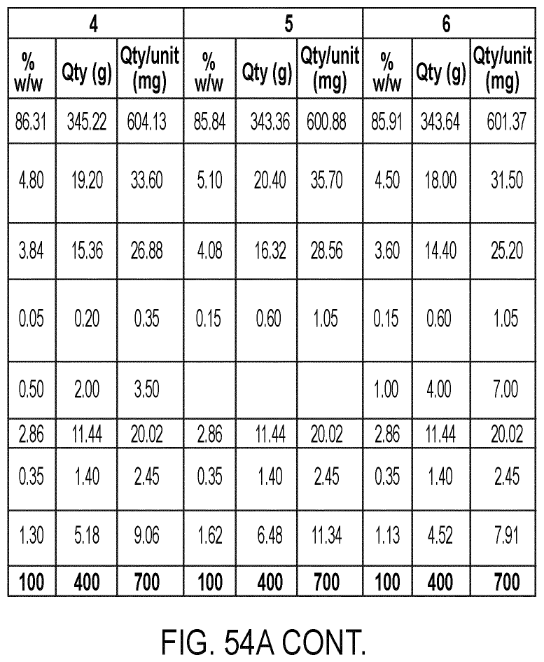

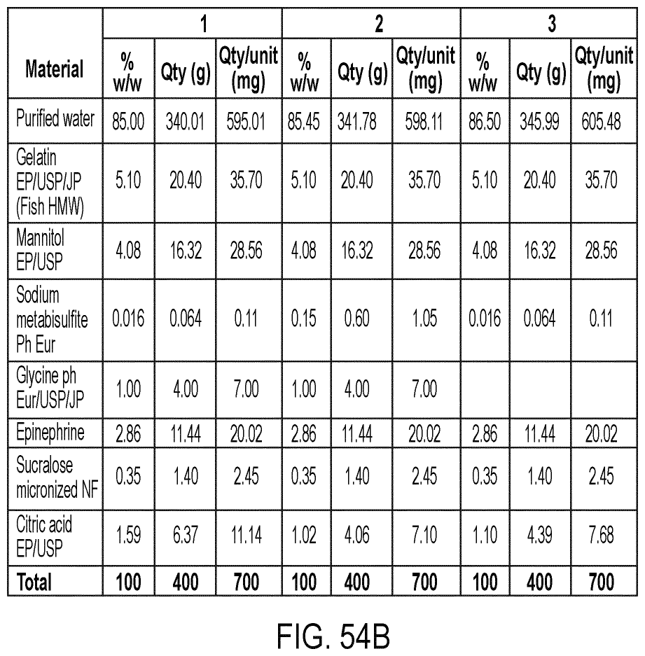

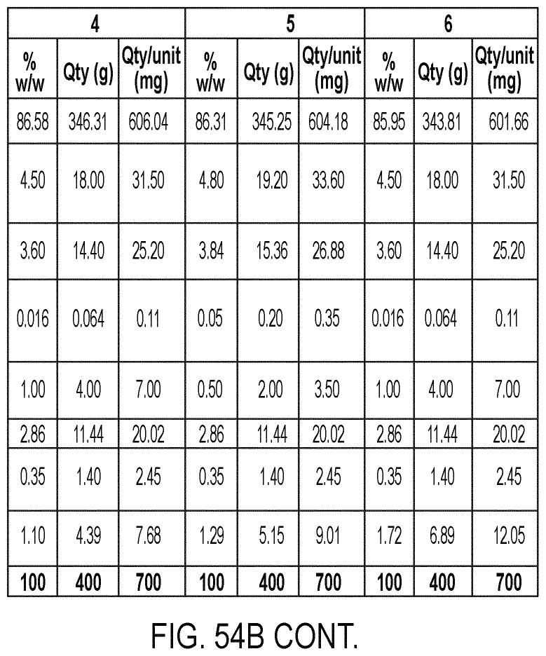

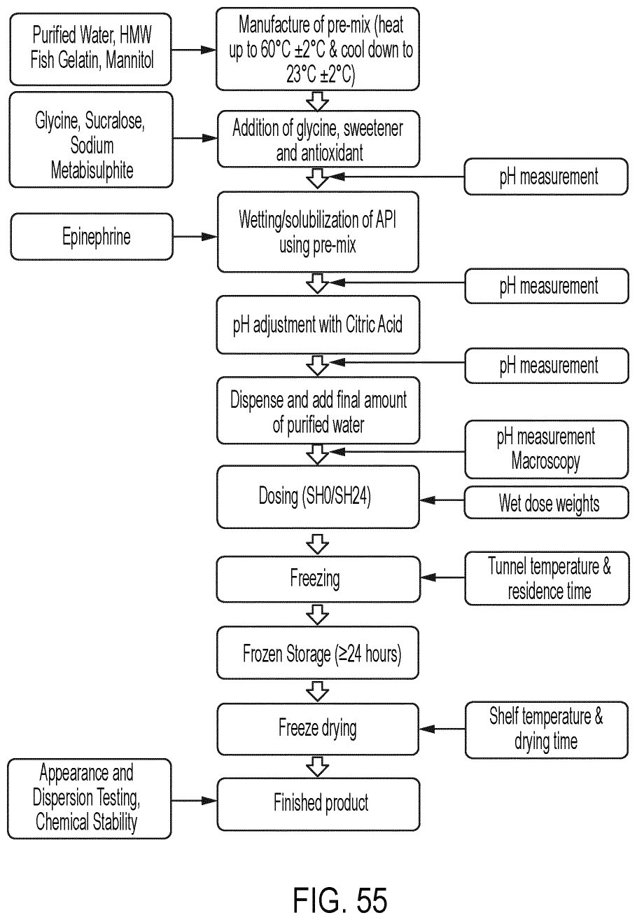

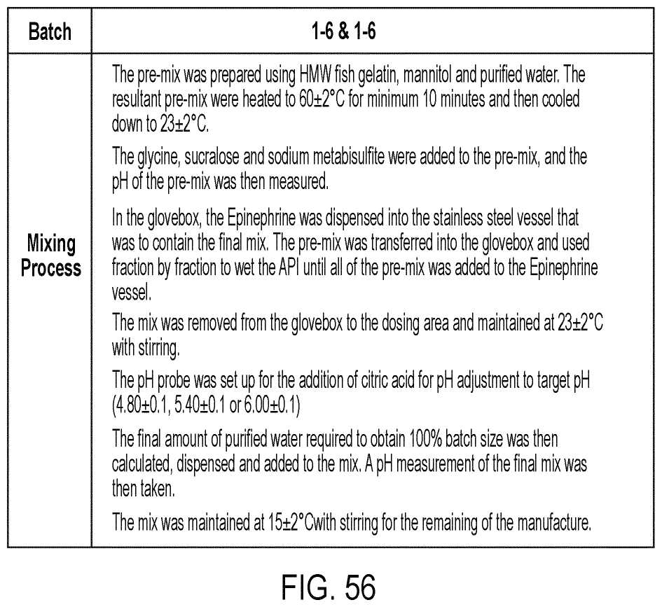

FIGS. 54A-B includes a more detailed list of the formulations used in this Study. FIG. 55 includes a manufacturing flow diagram for the batches made in this Study. The preparation of the mixes was conducted as described in FIG. 56. It was noted during pH adjustment of Cycle 1, batches 3, 4, and 6 that a small amount of white particles were present in the mix. This is likely due to the relatively higher target pH of these batches, at which the API is less soluble and thus takes longer to dissolve. No particles of this nature were observed at later macroscopic inspection time points. During API wetting of Cycle 2, batch 1, it was observed that the volume of premix was higher than that of other batches. Balance printouts relating to the preparation of this batch were checked and the initial amount of purified water dispensed for this batch was found to be incorrect. Consequently, this mix was disposed of and the batch repeated to provide correct formulation. This did not influence the outcome of this study.

Batches were dosed either immediately after the end of mixing (SH0), or after 24 hours of solution hold (SH24). An error occurred after dosing of Cycle 1, batches 1-6 at the 0-hour solution hold; the water bath temperature was set to −20° C. following dosing of the SH0 trays, as opposed to 15±2° C. as specified. The SH24 trays were ten dosed at a temperature of 20.4° C. This effect of this deviation was to be evaluated by comparison of stability data between batches from Cycle 1 (dosed at ˜20° C.) and Cycle 2 (dosed at ˜15° C.). No issues were encountered during dosing, although a low incidence of slight wedging was observed due to the pocket geometry and fill size used; 700 mg wet fill into a 750 mg pocket.

No issues were encountered during freezing. The trays were frozen at −90° C. with a freeze tunnel residence time of 3 minutes and 15 seconds. The period during which the product is transferred from the freeze tunnel to a freezer and stored below −15° C. (freezer set up at approximately −25° C.) until loading into the freeze dryer is referred to as the frozen hold (FH) time. All batches had a minimum frozen hold of 24 hours. Batches were dried at 0° C. for 18 hours. Following review of the freeze-drying trace, no problems were recorded. All batches were sealed under 5 bar of pressure at 185° C. with a dwell time of 1 second. All blisters tested passed the vacuum leak test with no observed shrinkage, indicating that the sealing conditions employed were suitable for this product.

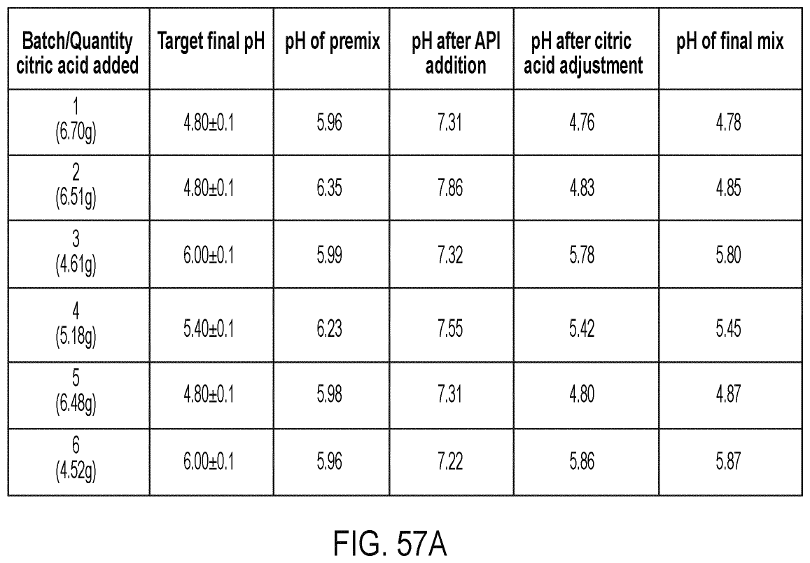

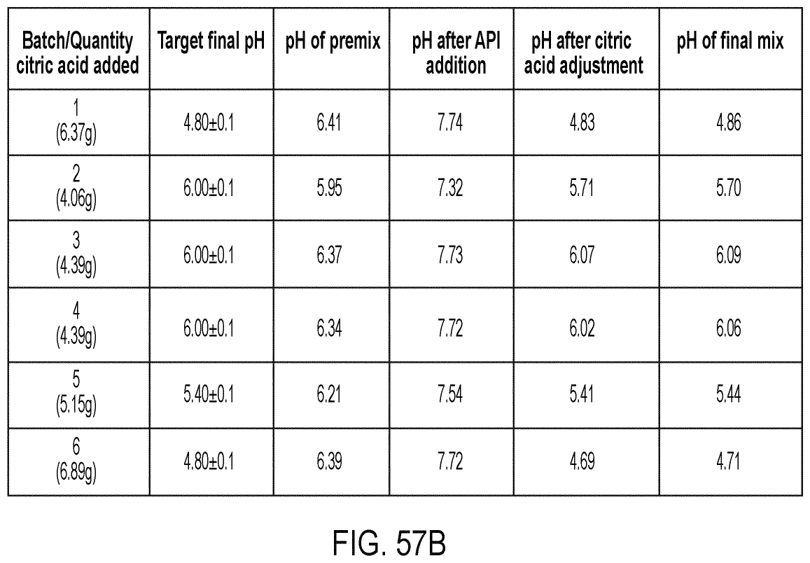

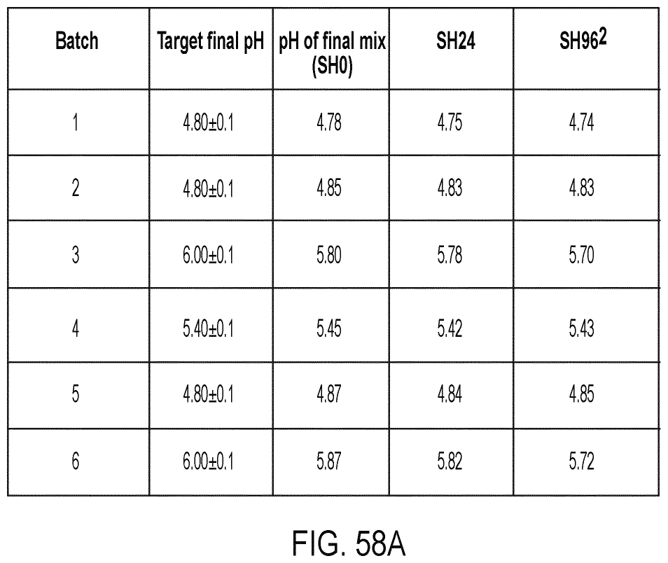

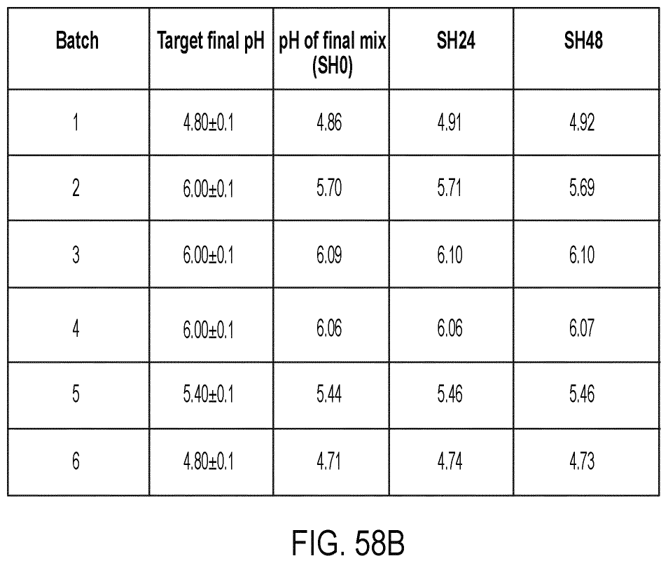

Citric acid was used to adjust the pH of the mixes. The pH measurements that were taken for each batch at different stages of mix preparation are shown in FIGS. 57A (cycle 1)-B (cycle 2). The pH was successfully adjusted to target pH for all batches bar Cycle 1, batches 3 and 6 and Cycle 2, batch 2. These batches were adjusted to slightly below target pH; this is not deemed to have a significant detrimental impact on the outcomes of this study. The pH of each batch was also monitored at the specified solution hold time points. The results for are provided in FIGS. 58A (cycle 1) and 58B (cycle 2). Results of pH monitoring during solution holding periods show that the pH of each mix remains largely stable with regards to the respective final mix (SH0) measurement.

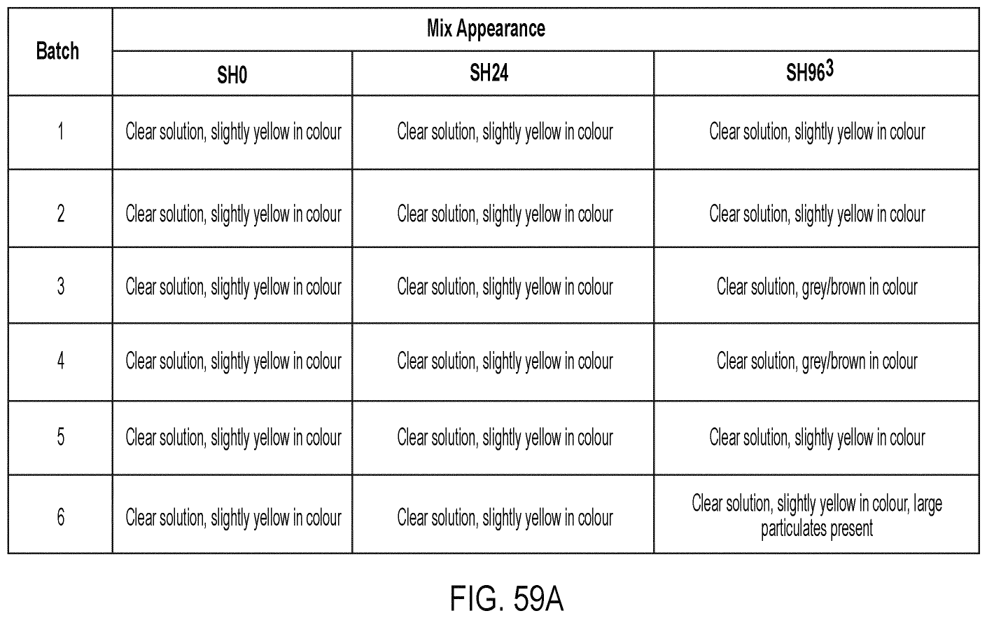

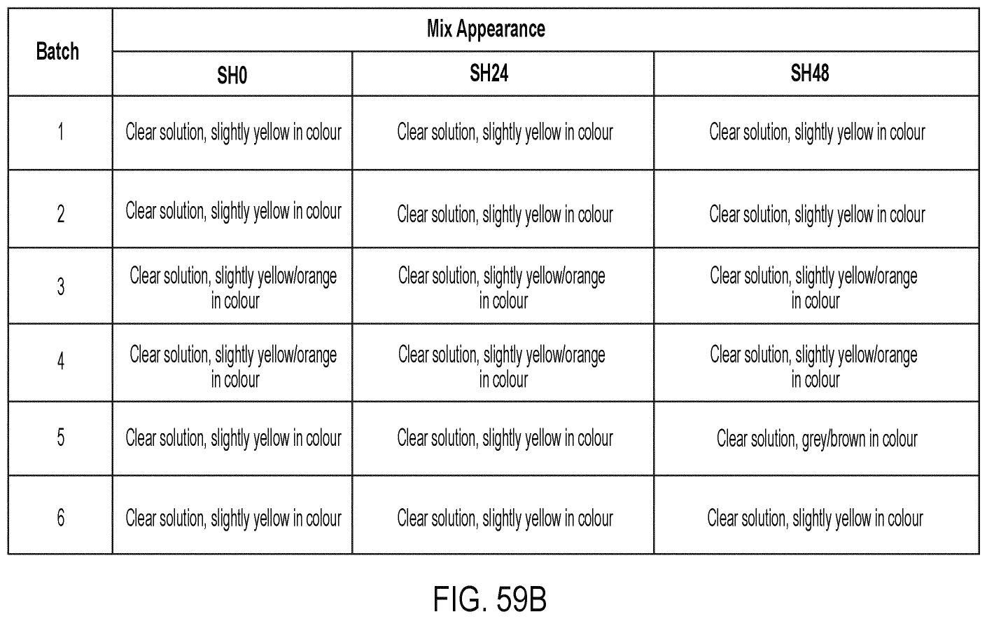

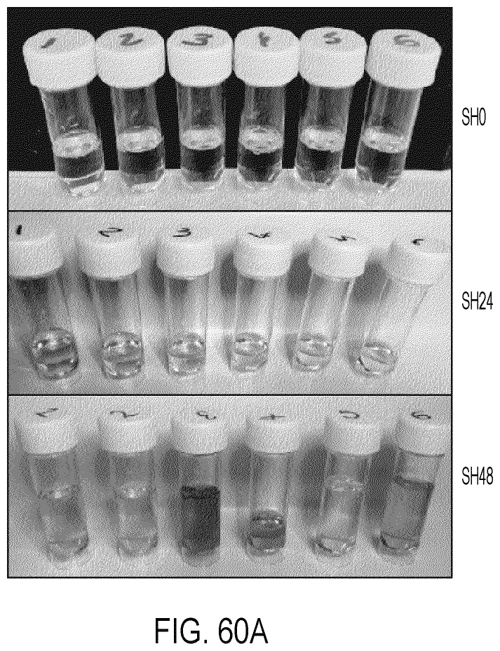

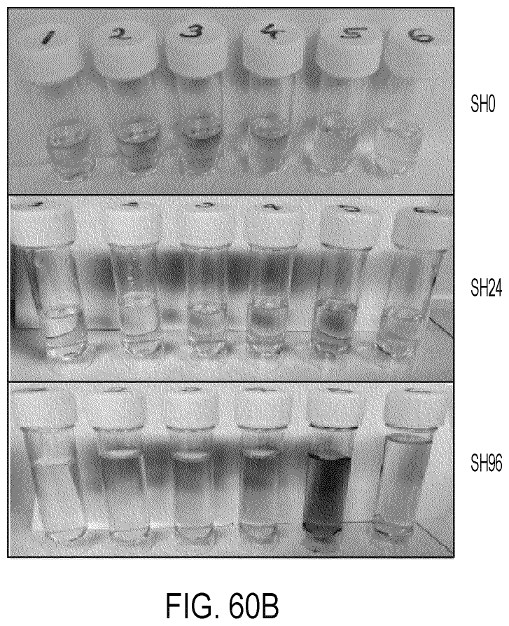

The appearance of the solutions were recorded at the end of mixing (SH0), after approximately 24 hours stirring (SH24) and after approximately 48 hours of stirring (SH48). The observations recorded for each mix are summarized in FIGS. 59A (cycle 1), 59B (cycle 2), 60A (cycle 1), and 60B (cycle 2). A significant color change was evident for batches 3-4 of cycle 1 after 96 hours of solution hold. Slight darkening and formation of large particulates was also observed for batch 6 of cycle 1 at this time point. These batches were adjusted to relatively higher pH levels and had relatively high/moderate levels of sodium metabisulfite. The darker appearance of these samples is indicative of Epinephrine instability/oxidation, suggesting that he antioxidant is more effective at lower pH levels. There is no evident color change over time for the remaining batches. A significant color change was evident for batch 5 after 48 hours of solution hold. This batch was formulated at pH 5.40, with 0.05 w/w sodium metabisulfite; a similar formulation evaluated in Cycle 1 (batch 4) also yielded comparable results, supporting conclusions that the sodium metabisulfite antioxidant functions better at lower pH levels. Batches 3-4 of Cycle 2 were also noted to be slightly orange in color on completion of mixing, although this color remained largely consistent throughout the hold period. These batches were adjusted to higher pH levels (pH 6.00) and had relatively low levels of sodium metabisulfite; the difference in the color of these batches may be indicative of some Epinephrine oxidation having occurred.

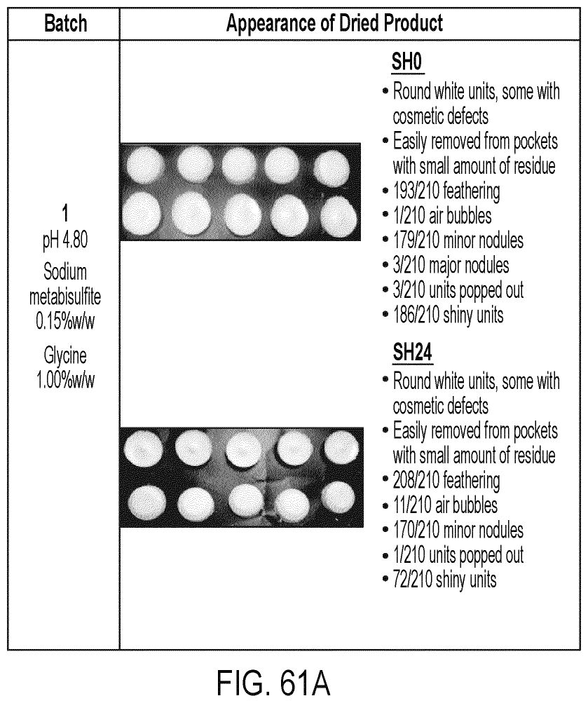

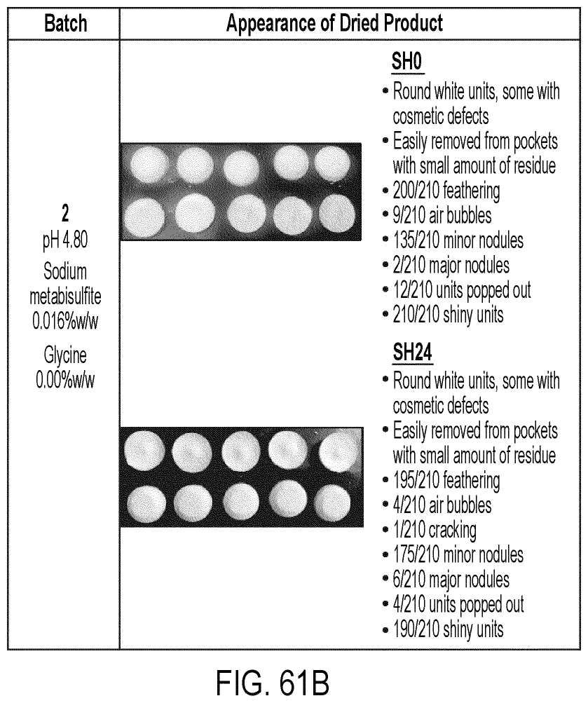

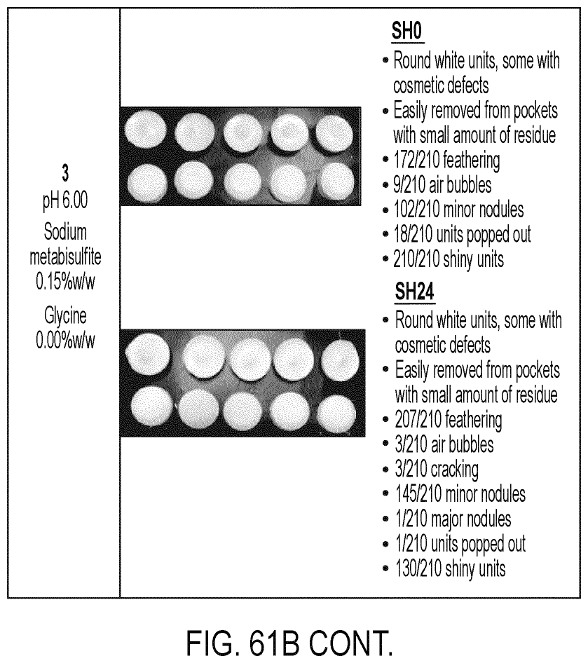

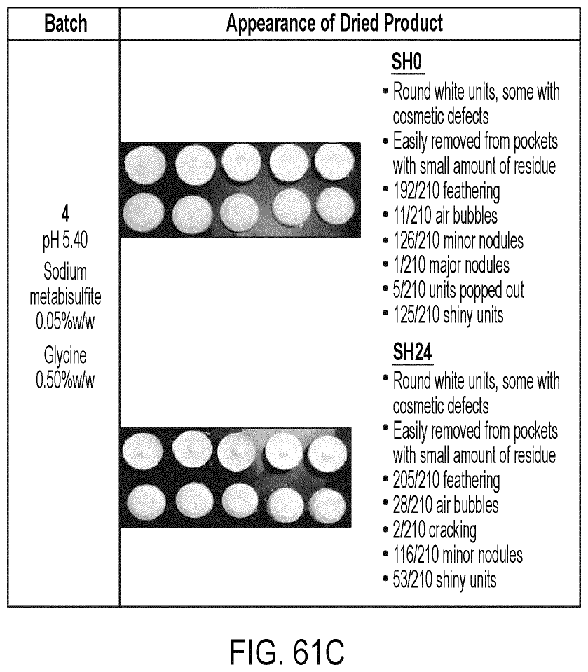

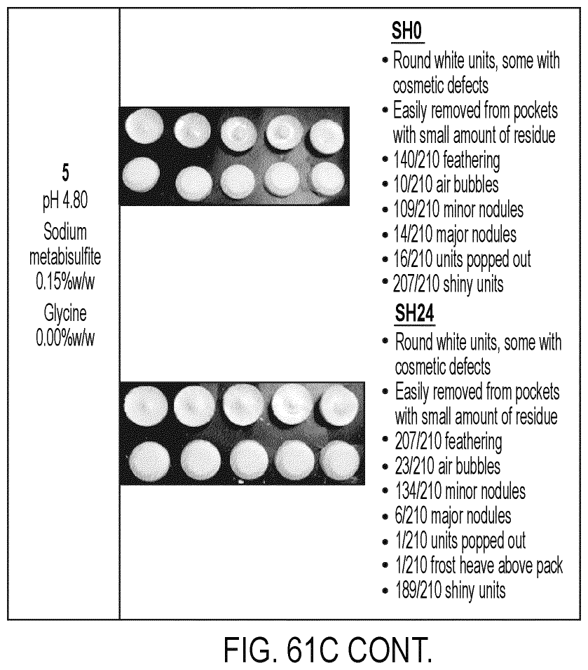

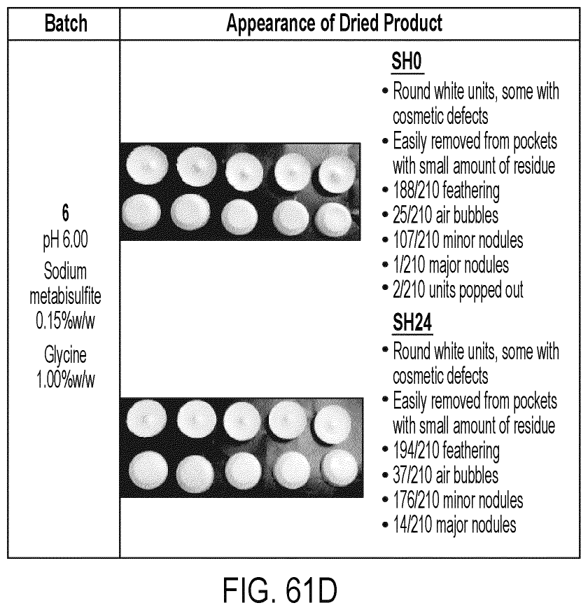

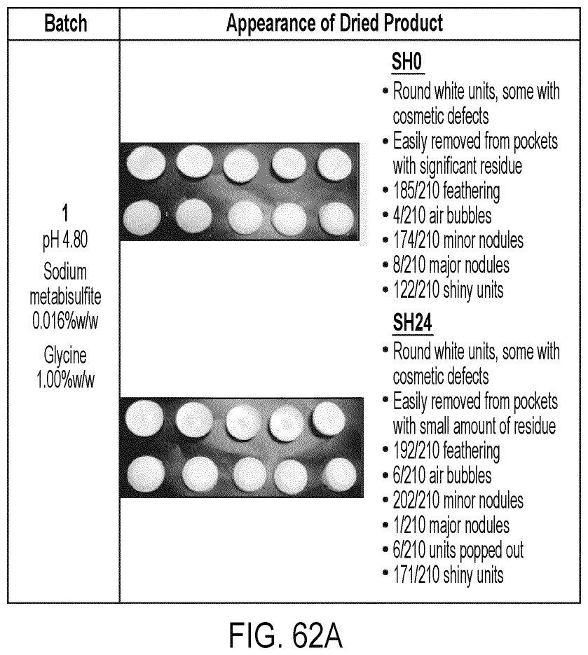

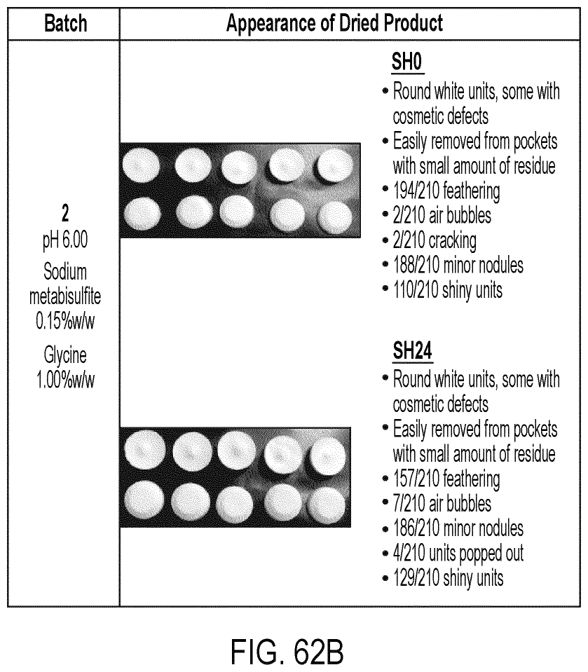

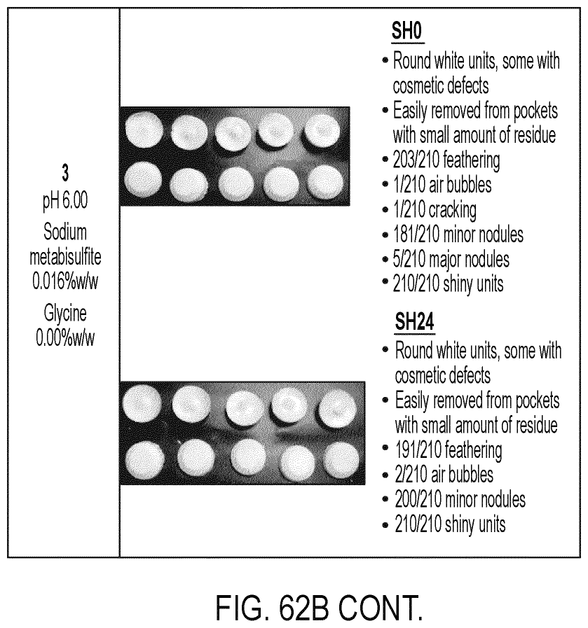

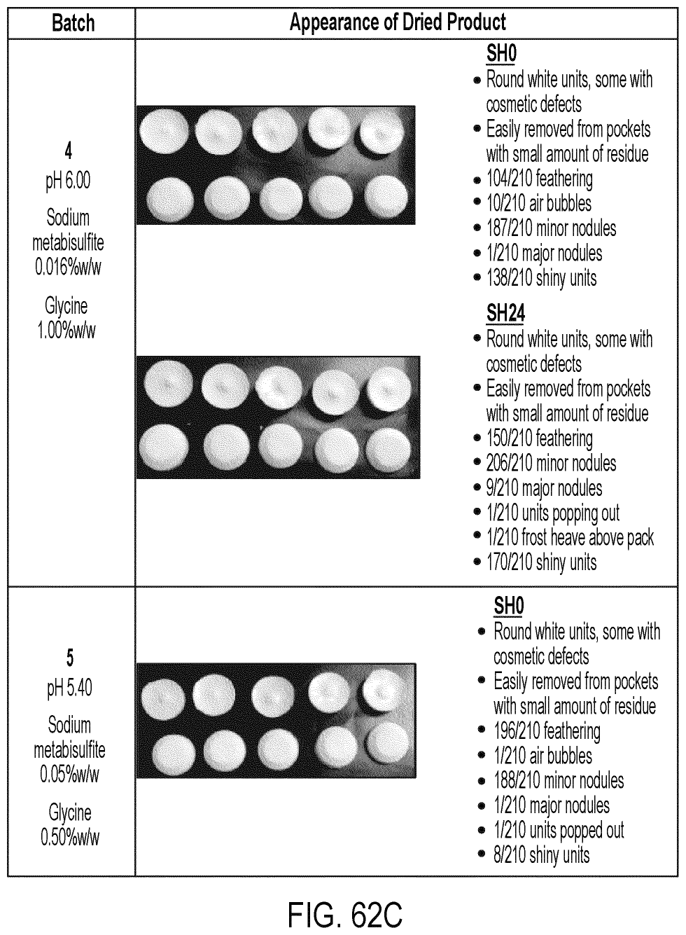

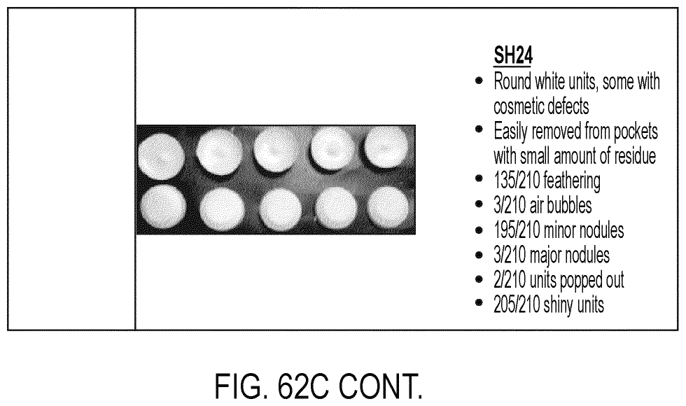

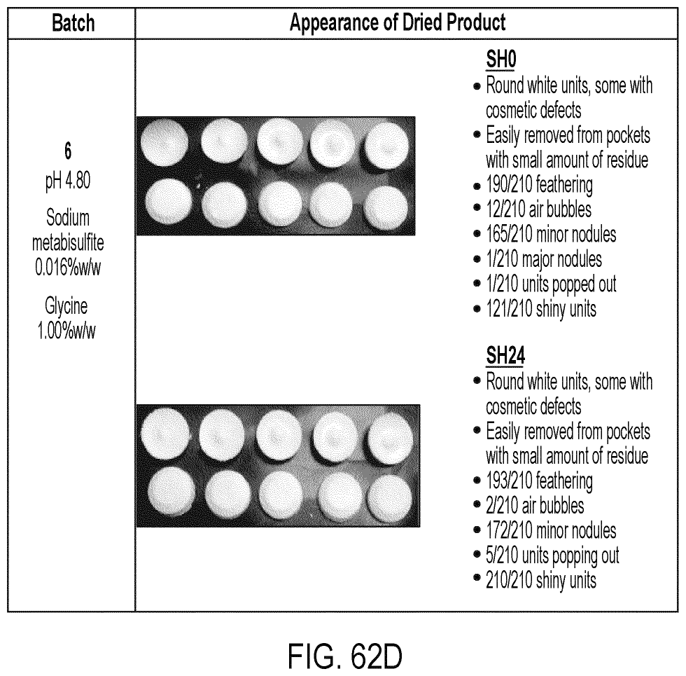

All batches were inspected and observations recorded in the batch manufacturing records. The appearance was considered good if the units were circular, freeze-dried tablets that could be removed from packaging without breaking and were generally free of defects. FIGS. 61A-D provide the finished product observations for Cycle 1, batches 1-6. FIGS. 62A-D provide the finished product observations for Cycle 2, batches 1-6. Overall unit appearance for all batches was suboptimal, with all batches producing round white units with a high incidence of cosmetic defects noted. Feathering was observed across all sub-batches; this is likely due to bench-scale related processing factors and would be expected to be eliminated at a larger manufacturing scale. Nodules were also observed across all sub-batches; this is a common observation when using fish gelatin and is likely to be a result of the bench-scale freezing process. Due to the pocket geometry that was required to be used in order to be able to seal the unit blisters, a small proportion of minor wedge shaped units were seen across all sub-batches. All batches dosed a wet fill weight of 700 mg into a 750 mg blister pocket.

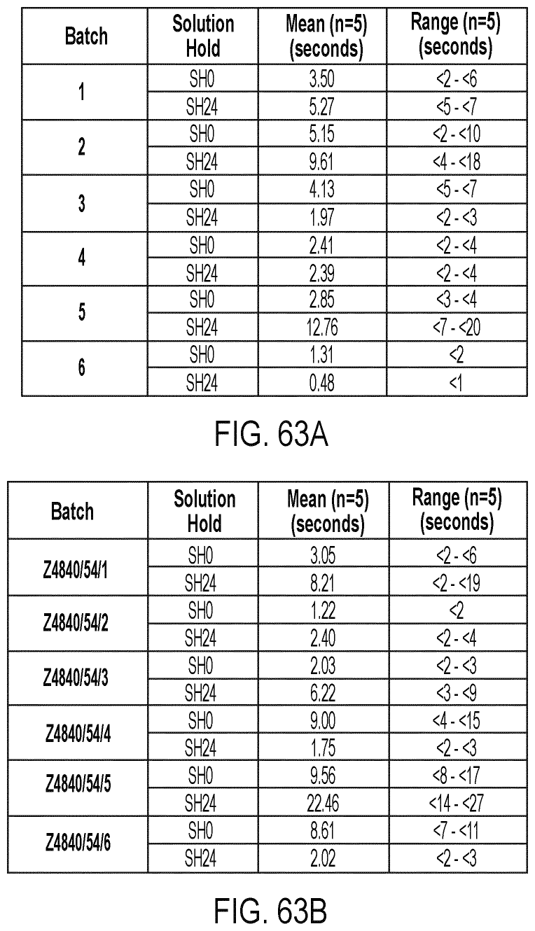

The dispersion time results for all batches are summarized in FIGS. 63A (cycle 1) and 63B (cycle 2). All batches except Cycle 1, batch 5 (SH24) and Cycle 2, batch 5 (SH24) dispersed within acceptable limit of less than or equal to 10 seconds. Batches formulated at higher pH levels generally had faster dispersion times. This indicates that the quantity of citric acid within the formulation may influence the finished product dispersion rate. The inclusion of glycine in the formulation did not appear to have any additional adverse effect on the dispersion profile of the units.

Selected sub-batches were evaluated with regards to the results of in-process and finished product testing, and knowledge gained from previous stability studies. The selected batches included those representing center-points of the study, and those with the highest levels of sodium metabisulfite. Sealed blisters of sub-batches deemed to have optimal characteristics, as well as those representing center-points in the experimental design, underwent stability studies. Blisters were to be stored under ambient conditions for up to 21 days to evaluate real time stability characteristics and determine whether previous chemical stability concerns were alleviated in the absence of aggressive temperatures. Following review of initial timepoint data, it was agreed to cease all analytical testing on all batches manufactured during this study due to inadequate chemical stability profiles.

A heat stress study was also performed; units were stored at 60° C. for 21 days to emulate longer-term stability conditions. The influence of numerous formulation factors on unit appearance and shrinkage was evaluated. Additionally, two EpiPen® and four Emerade® auto-injector pen(s) were stored under heat stress conditions to provide a reference point and allow stability comparison.

The following Table 8 provides the assay results of composite samples at the initial timepoint (Day 0) of the solution-based formulations dosed at SH24:

TABLE 8

| | | | Impurity F | |
|---|---|---|---|---|
| | | | (Relative | |
| | | | Retnetion time | |
| | Epinephrine | Sodium | to Epinephrine | Total Impurities |
| | (% Label | metabisulfite | (“RRT”)~0.8) | (Unknown + |
| Sample | Claim) | (% Label Claim)* | (%) | Impurity F) (%) |
| Cycle 1, sub- | 100.7 | 2.6 | none detected | 1.21 |
| batch 1 (pH 4.8) | | | (<0.02%) | |
| Cycle 1, sub- | 98.3 | 33.8 | 0.99 | 1.39 |
| batch 3 (pH 6.0) | | | | |
| Cycle 1, sub- | 98.2 | none detected | none detected | 0.95 |
| batch 4 (pH 5.4) | | (<0.02%) | (<0.02%) | |
| Cycle 1, sub- | 99.8 | 1.4 | 0.19 | 0.98 |
| batch 5 (pH 4.8) | | | | |
| Cycle 1, sub- | 100.1 | 42.2 | 1.43 | 1.99 |
| batch 6 (pH 6.0) | | | | |
| Cycle 2, sub- | 99.5 | 44.7 | 1.19 | 1.79 |
| batch 2 (pH 6.0) | | | | |
| Cycle 2, sub- | 100.8 | none detected | none detected | 1.01 |
| batch 5 (pH 5.4) | | (<0.02%) | (<0.02%) | |

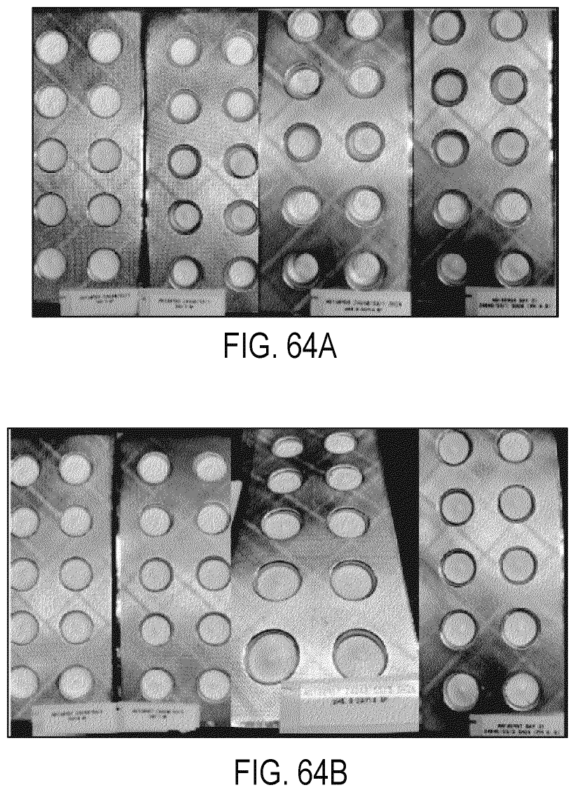

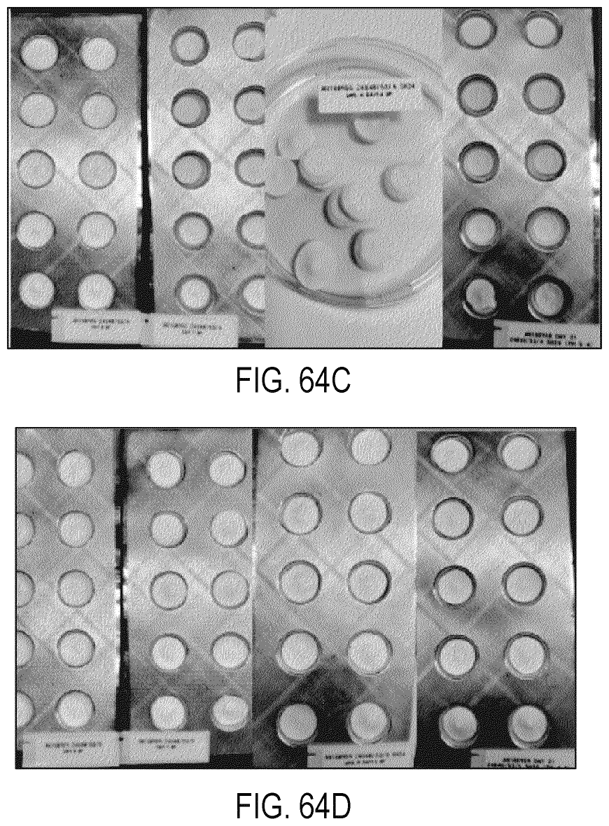

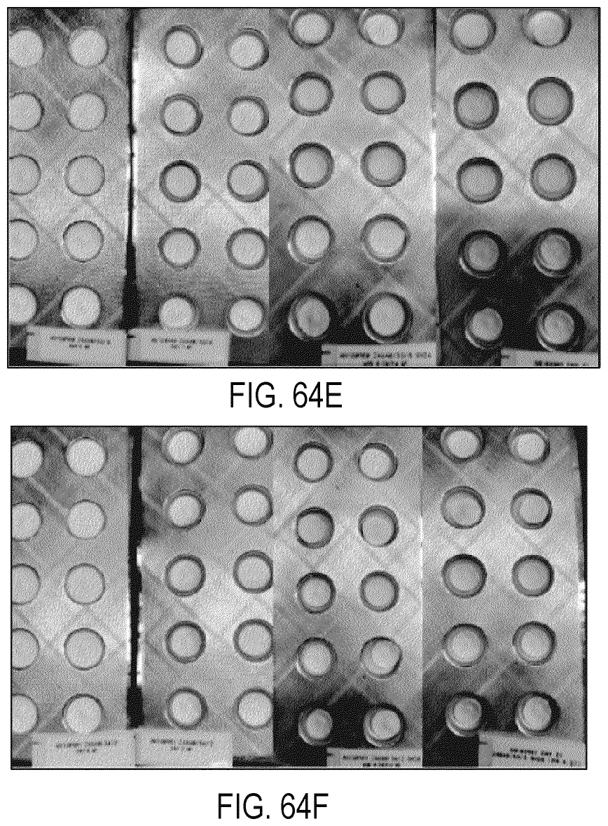

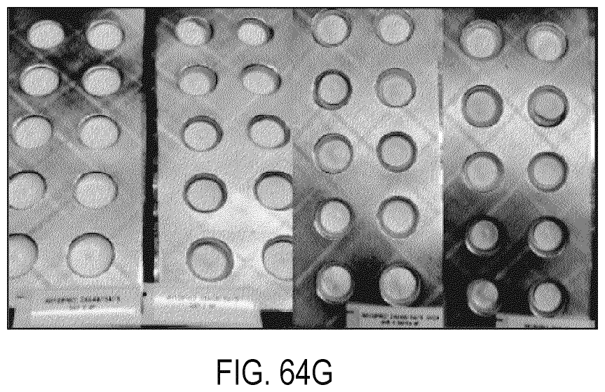

FIG. 64A shows the appearance of Cycle 1, batch 1 following 21 days of heat storage. The unit properties for Cycle 1, sub-batch 1 were acceptable at the initial time point, however discoloration and shrinkage were evident following storage. The inclusion of 1.00% w/w glycine does not appear to be beneficial for this formulation. Analytical data shows that whilst the assay recovery is within acceptable limits, the Sodium metabisulfite has already been largely consumed. Based on prior knowledge from previous tests, it is anticipated that assay values would decline rapidly, and be accompanied by a vast increase in the accumulation of impurities following complete depletion of the antioxidant. These results indicate that this formulation is not well protected against oxidation and may not have a suitable shelf life. Consequently, any further analytical testing on this batch was cancelled as previously discussed.

FIG. 64B shows the appearance of Cycle 1, batch 3 following 21 days of heat storage. The unit properties for Cycle 1, sub-batch 3 were acceptable at the initial time point, however discoloration and slight shrinkage were evident following storage. As this batch did not contain glycine, the lesser degree of shrinkage for this batch also confirms the lack of benefit of its inclusion in other formulations. Analytical data shows that whilst the assay recovery is within acceptable limits, the Sodium metabisulfite has already been largely consumed, although to a lesser extent than that seen in batches formulated at lower pH levels. It was also noted that the level of Impurity F formation is greater at the higher pH level. This suggests that the pH of the formulation influences the rate of Sodium metabisulfite consumption, and the rate of Impurity F formation. As with all batches and based on prior knowledge, it is anticipated that assay values would decline rapidly, and be accompanied by a vast increase in the accumulation of impurities following complete depletion of the antioxidant. These results indicate that this formulation is not well protected against oxidation and may not have a suitable shelf life. Consequently, any further analytical testing on this batch was cancelled as previously discussed.

FIG. 64C shows the appearance of Cycle 1, batch 4 following 21 days of heat storage. The unit properties for Cycle 1, sub-batch 4 were acceptable at the initial time point, however discoloration and shrinkage were evident following storage. The inclusion of 0.50% w/w glycine does not appear to be beneficial for this formulation. Analytical data shows that whilst the assay recovery is within acceptable limits, the Sodium metabisulfite has already been completely consumed. As with all batches and based on prior knowledge, it is anticipated that assay values would decline rapidly, and be accompanied by a vast increase in the accumulation of impurities in the absence of the antioxidant. These results indicate that this formulation is not well protected against oxidation and may not have a suitable shelf life. Consequently, any further analytical testing on this batch was cancelled as previously discussed.

FIG. 64D shows the appearance of Cycle 1, batch 5 following 21 days of heat storage. The unit properties for Cycle 1, sub-batch 5 were acceptable at the initial time point, however discoloration and shrinkage were evident following storage. There was a lesser degree of shrinkage compared to units from other batches. As this batch did not contain glycine, this also confirms the lack of benefit of its inclusion in other formulations. Analytical data shows that whilst the assay recovery is within acceptable limits, the Sodium metabisulfite has already been largely consumed. As with all batches and based on prior knowledge (PR703660), it is anticipated that assay values would decline rapidly, and be accompanied by a vast increase in the accumulation of impurities following complete depletion of the antioxidant. These results indicate that this formulation is not well protected against oxidation and may not have a suitable shelf life. Consequently, any further analytical testing on this batch was cancelled as previously discussed.

FIG. 64E shows the appearance of Cycle 1, batch 6 following 21 days of heat storage. The unit properties of Cycle 1, sub-batch 6 were acceptable at the initial time point, however discoloration and shrinkage were evident following storage. The inclusion of 1.00% w/w glycine does not appear to be beneficial for this formulation. Analytical data shows that whilst the assay recovery is within acceptable limits, the Sodium metabisulfite has already been largely consumed, although to a lesser extent than that seen in batches formulated at lower pH levels. It was also noted that the level of Impurity F formation is greater at the higher pH level. This suggests that the pH of the formulation influences the rate of Sodium metabisulfite consumption, and the rate of Impurity F formation. As with all batches and based on prior knowledge, it is anticipated that assay values would decline rapidly, and be accompanied by a vast increase in the accumulation of impurities following complete depletion of the antioxidant. These results indicate that this formulation is not well protected against oxidation and may not have a suitable shelf life. Consequently, any further analytical testing on this batch was cancelled as previously discussed.

FIG. 64F shows the appearance of Cycle 1, batch 2 following 21 days of heat storage. The unit properties of Cycle 2, sub-batch 2 were acceptable at the initial time point, however discoloration and shrinkage were evident following storage. The inclusion of 1.00% w/w glycine does not appear to be beneficial for this formulation. Analytical data shows that whilst the assay recovery is within acceptable limits, the Sodium metabisulfite has already been largely consumed, although to a lesser extent than that seen in batches formulated at lower pH levels. It was also noted that the level of Impurity F formation is greater at the higher pH level. This suggests that the pH of the formulation influences the rate of Sodium metabisulfite consumption, and the rate of Impurity F formation. As with all batches and based on prior knowledge, it is anticipated that assay values would decline rapidly, and be accompanied by a vast increase in the accumulation of impurities following complete depletion of the antioxidant. These results indicate that this formulation is not well protected against oxidation and may not have a suitable shelf life. Consequently, any further analytical testing on this batch was cancelled as previously discussed.

FIG. 64G shows the appearance of Cycle 1, batch 5 following 21 days of heat storage. The unit properties of Cycle 2, sub-batch 5 were acceptable at the initial time point, however discoloration and shrinkage were evident following storage. The inclusion of 0.50% w/w glycine does not appear to be beneficial for this formulation. Comparison of this batch and Cycle 1, sub-batch 4 (center-point formulations) allows evaluation of the deviation regarding dosing temperature. These formulations were dosed at ˜20° C. and ˜15° C. respectively. Epinephrine assay recovery is slightly lower (98.2% label claim) for the batch dosed at the higher temperature, however this may not be a true result considering bench scale processing factors and analytical variance. Analytical data shows that whilst the assay recovery is within acceptable limits, the Sodium metabisulfite has already been completely consumed. As with all batches and based on prior knowledge (PR703660), it is anticipated that assay values would decline rapidly, and be accompanied by a vast increase in the accumulation of impurities in the absence of the antioxidant. These results indicate that this formulation is not well protected against oxidation and may not have a suitable shelf life. Consequently, any further analytical testing on this batch was cancelled as previously discussed.

Additionally, two EpiPen® and four Emerade® (Epinephrine) auto-injector pen(s) were stored under heat stress conditions to provide a reference point and allow stability comparison between the solution-based Epinephrine formulation and an Epinephrine auto-injector (the most common format currently used for the same indication). An objective of the solution-based Epinephrine product development is to provide a competitor to the EpiPen®. Due to global stock issues, the two EpiPen® products that were able to be sourced had differing packaging/batch numbers (Meda® and Mylan® branding) and expiry dates, thus preventing accurate comparison between the pens. Consequently, only results from days 7 and 14 could be obtained for these pens. The following Table 9 provides the assay results for the Epipen® and Emerade® Auto-injectors:

TABLE 9

| | | Sodium | Impurity F | Total Impurities |
|---|---|---|---|---|
| | Epinephrine (% | metabisulfite | (RRT~0.8) | (Unknown + |
| Sample | Assay Loss) | (% Assay Loss) | (%) | Impurity F) (%) |
| Meda ® EpiPen ® | 0.00 | 0.00 | 5.66 | 10.66 |
| Day 0 | | | | |
| Meda ® EpiPen ® | 1.32 | 14.81 | 12.79 | 21.99 |
| Day 7 | | | | |
| Mylan ® EpiPen ® | 0.00 | 0.00 | 2.18 | 16.15 |
| Day 0 | | | | |
| Mylan ® EpiPen ® | 14.38 | 21.05 | 21.52 | 33.48 |
| Day 14 | | | | |
| Emerade ® | 0.00 | 0.00 | 0.80 | 6.10 |
| Day 0 | | | | |
| Emerade ® | 2.58 | 53.85 | 4.81 | 12.17 |
| Day 7 | | | | |
| Emerade ® | 4.31 | 73.08 | 8.06 | 14.28 |
| Day 14 | | | | |
| Emerade ® | Poor peak shape (split | 7.69 | 9.02 | 15.14 |
| Day 21 | peak) for Epinephrine | (Possible anomalous | | |
| | observed in standard | result. One pen | | |
| | chromatograms attributed | analysed at each time | | |
| | to loss in column | point so variation in | | |
| | performance. Assay | the individual | | |
| | result not reported. | performance of each | | |
| | | pen may vary, as well | | |
| | | as the behaviour of | | |
| | | the packaging under | | |
| | | aggressive conditions.) | | |

Two EpiPen autoinjectors were also put on storage to allow comparison of the stability profiles between solution-based Epinephrine formulation units and this format, and to provide a reference point against the most common dosage format currently marketed for the same indication. The data shows trends of increasing assay and sodium metabisulfite loss over the periods of storage. These losses are also accompanied by increases in the levels of impurities. These trends mirror those observed in previous stability studies conducted on solution-based Epinephrine formulations disclosed above. However, a higher rate of assay decline from the initial timepoint to Day 7 is observed in the solution-based Epinephrine formulation units when compared to the EpiPen®, although assay results at Day 14 are comparable. Accumulation of impurities also follows this pattern. Additionally, the rate of Sodium metabisulfite consumption is also markedly more rapid in the solution-based Epinephrine formulations format. Analytical data indicates that the EpiPen® has superior chemical stability characteristics when compared to the evaluated solution-based Epinephrine formulation units.

Four Emerade® pens were also put on storage to allow comparison of the stability profiles between the solution-based Epinephrine formulation units and this format, and to provide a reference point against the most common dosage format currently marketed for the same indication. As observed during the solution-based Epinephrine formulations, analytical data shows that the assay recovery declines continuously throughout the 21 day study period. However, the rate of decline is markedly slower than that seen in the solution-based Epinephrine formulations. The rate of Sodium metabisulfite consumption is also lower. These results indicate that the Emerade® pen has superior stability. Comparison of the stability data between the two Epinephrine auto-injector products also suggests that the Emerade® pen is more stable than the EpiPen® with regards to the rate of assay loss. On review of the listed ingredients in the Emerade® pen, the inclusion of disodium edetate was noted. The enhanced stability of the Emerade® pen may be attributable to this excipient, which is known to function synergistically with the sodium metabisulfite antioxidant. The inclusion of disodium edetate as a potential improvement was assessed during future formulation studies for the Epinephrine products disclosed herein. The superior stability of the Emerade® pen may also be owed to the injectable solutions being able to be formulated at a lower pH (pH˜3.5), as well as degassed by purging with Nitrogen (or other inert gas). The act of displacing any oxygen within the formulations lends to obvious advantages in stability.

### Suspension-Based Formulation Study 1

The objective of this study was to manufacture and evaluate a suspension-based Epinephrine formulation ensuring that the target product profile and critical quality attributes are met and that competes with the current EpiPen® product is achieved. The formulations deemed to produce units with optimal characteristics were also set down on stability. The incorporation of API into one matrix (HMW fish gelatin) was to be assessed. A unit wet fill weight of 150 mg was used, providing a dosage strength of 5 mg or 20 mg for each batch (3.33% w/w and 13.33% w/w Epinephrine respectively). The proportion of gelatin included in the formulation was altered between the two strengths to account for the lesser solid content lower dosage strength. Critical physical attributes of the dried units were to be evaluated, including appearance and dispersion time. Sodium hydroxide was to be used as a pH modifier to assess the effect of pH on mix properties and finished units in terms of chemical stability. The inclusion of Disodium edetate (EDTA), known to act synergistically with the Sodium metabisulfite antioxidant, was also to be evaluated with regards to potential improvements in chemical stability.

For this study, HMW fish gelatin was used as the matrix former. Prior knowledge indicated that this matrix appeared to give better stability results, despite slightly greater incidence of cosmetic defects noted in previous studies compared to bovine gelatin. The proportion of the antioxidant Sodium metabisulfite in the formulation was included at the highest level previously evaluated (0.30% w/w) based on prior observations regarding its consumption during the manufacturing process. Inclusion of EDTA in these formulations was also to be evaluated to determine any enhancement of product stability. Two batches were also manufactured excluding both Sodium metabisulfite and EDTA to determine the requirement for these excipients when formulating as a suspension product. Batches were to be manufactured under light protection as in previous studies.

To allow assessment of suspension-based Epinephrine formulations, six batches were to be produced with the aim of:
Evaluating beneficial aspects of formulating at higher pH levels, thus having the majority of the drug in solid state, in terms of chemical stability and appearance of the finished product;
Assessing use of 150 mg fill weight and therefore differing ratios of solid state drug within the finished product on chemical stability;
Assessing numerous additional factors relating to the change from solution to suspension formulation; particle size, viscosity, microscopy, macroscopy, stoppage studies, requirement for homogenization;
Assessing changes in formulation factors, and stability profiles across the suspension hold time (up to 24 hours); and
Assessing potential synergistic enhancement of the Sodium metabisulfite (antioxidant) performance in relation to chemical stability.

A placebo formulation was also to be produced for analytical purposes. No testing was performed on this batch, as such it will not be discussed. The mixes were to be dosed at the end of mixing (0 hours suspension hold, SH0), and after 24 hours of suspension hold (SH24). The product had a minimum of 24 hours frozen hold (FH) prior to freeze drying. The batches were freeze dried at a shelf temperature of 0° C. on a 6 hour cycle. Appearance and dispersion testing was to be performed on units obtained from all batches. Appropriate samples of each batch were to be analyzed for chemical stability. The following Table 10 includes the formulations for this suspension-based formulation study:

TABLE 10

| | | | | | Sodium | pH (Sodium |
|---|---|---|---|---|---|---|
| | Sub- | Epinephrine | Gelatin | EDTA | metabisulfite | hydroxide for |
| Batch | batch | (% w/w) | (% w/w) | (% w/w) | (% w/w) | adjustment) |
| 1 | 1 | 3.33 | 4.80 | 0.05 | 0.30 | — |
| | | (5 mg) | | | | |
| | 2 | 13.33 | 4.30 | 0.05 | 0.30 | — |
| | | (20 mg) | | | | |
| | 3 | 3.33 | 4.80 | 0.05 | 0.30 | 8.5 ± 0.1 |
| | | (5 mg) | | | | |
| | 4 | 13.33 | 4.30 | 0.05 | 0.30 | 8.5 ± 0.1 |
| | | (20 mg) | | | | |
| | 5 | 3.33 | 4.80 | 0.00 | 0.00 | — |
| | | (5 mg) | | | | |
| | 6 | 3.33 | 4.80 | 0.00 | 0.00 | 8.5 ± 0.1 |
| | | (5 mg) | | | | |

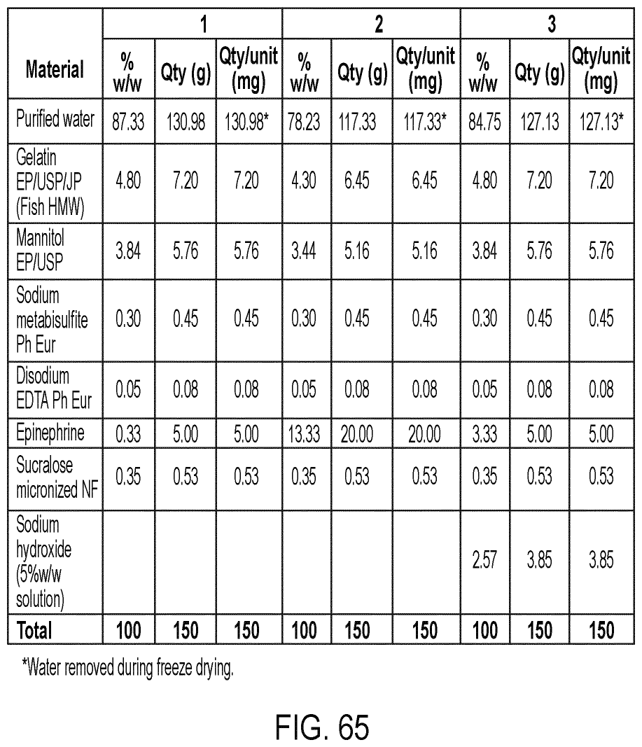

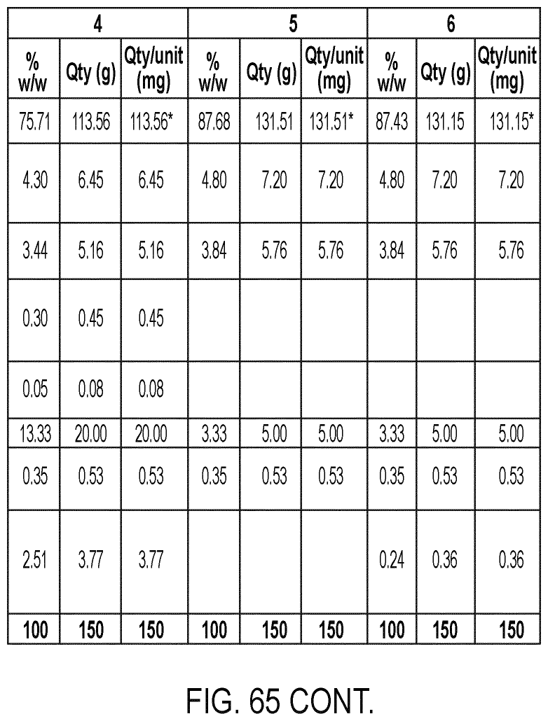

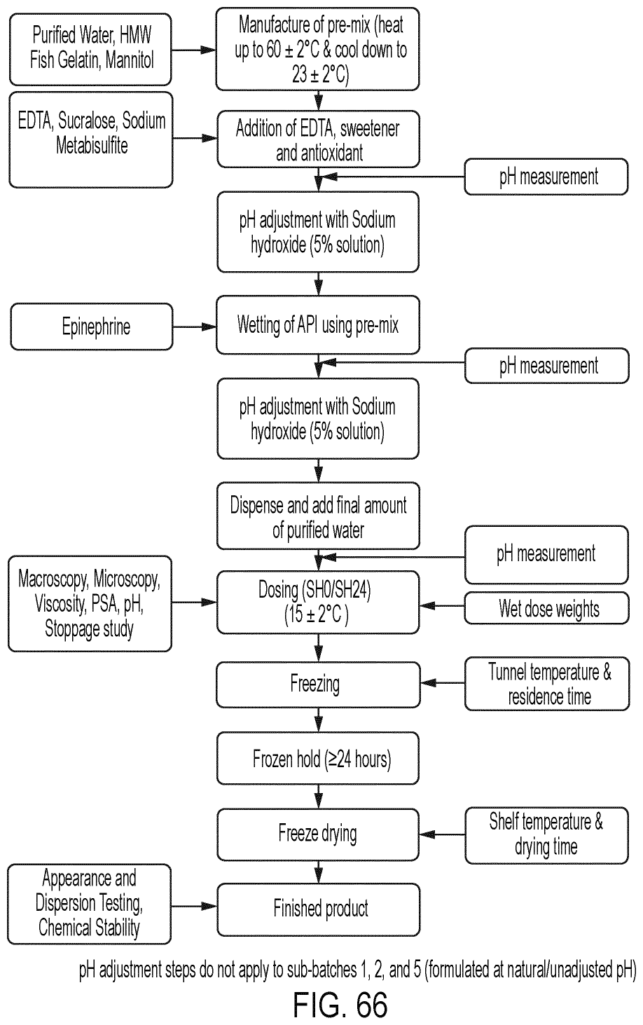

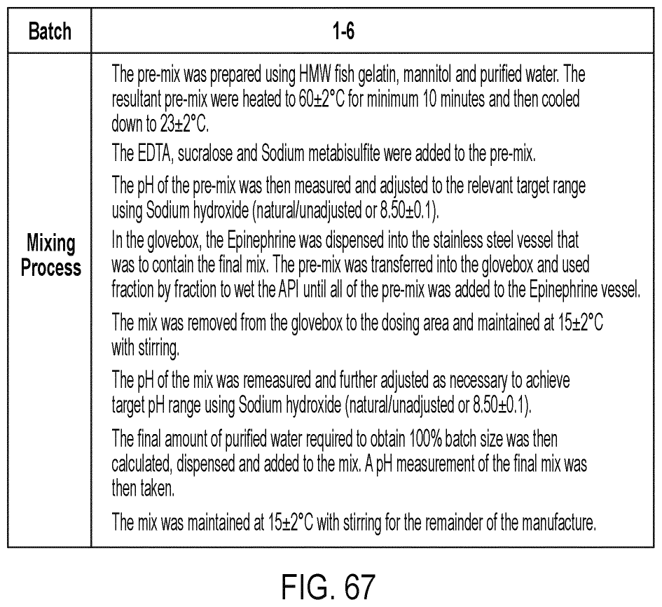

More detail for the suspension formulations tested in this Study can be found in FIG. 65. FIG. 66 is a manufacturing flow chart for the various batches produced in this Study. The preparation of the mixes was conducted as described in FIG. 67. No issues were encountered during API addition. Completion of the mixing process produced a white suspension for each respective batch. During pH adjustment of the premix of sub-batch 3, the pH was adjusted above the target range (˜8.5) in error, to pH 8.88. The final pH reading for this batch following API and final water addition was 8.82. The impact of this deviation on chemical stability will be assessed when comparing data obtained from stability studies.

Batches were dosed immediately after the end of mixing (SH0), and after 24 hours of suspension hold (SH24). A stoppage study was also performed at the initial timepoint to assess factors such as settling of the suspension during the dosing process, and the effect on content uniformity. No issues were encountered during dosing, although a low incidence of minor wedging was observed. This is likely a result of bench scale processing factors (i.e., hand dosing).

No issues were encountered during freezing. The trays were frozen at −70° C. with a freeze tunnel residence time of 3 minutes and 15 seconds. The period during which the frozen product is transferred from the freeze tunnel to a freezer and stored below −15° C. (freezer set up at approximately −25° C.) until loading into the freeze dryer is referred to as the frozen hold (FH) time. Frozen hold can be important as it can allow any amorphous mannitol to crystallize giving added strength to the tablet. All batches had a minimum frozen hold of 24 hours. Batches were freeze dried at 0° C. for 6 hours. On drying of sub-batches 1-5 from the 0-hour suspension hold timepoint (SH0), it was noted that the drying cycle had not run to specification (shelf temperature not controlled at 0° C.). This was a result of an equipment issue. Review of the freeze drying trace indicates that the shelf temperatures were below the specified parameters. The impact of this deviation was to be evaluated as part of the finished product inspection procedure.

All batches were sealed under 5 bar of pressure at 180 C with a dwell time of 1 second. All blisters tested passed the vacuum leak test with no observed shrinkage, indicating that the sealing conditions employed were suitable for this product. Observations during the vacuum leak test identified a high occurrence of the unit frost heave touching the lidding foil. This was noted to be a very minor issue.

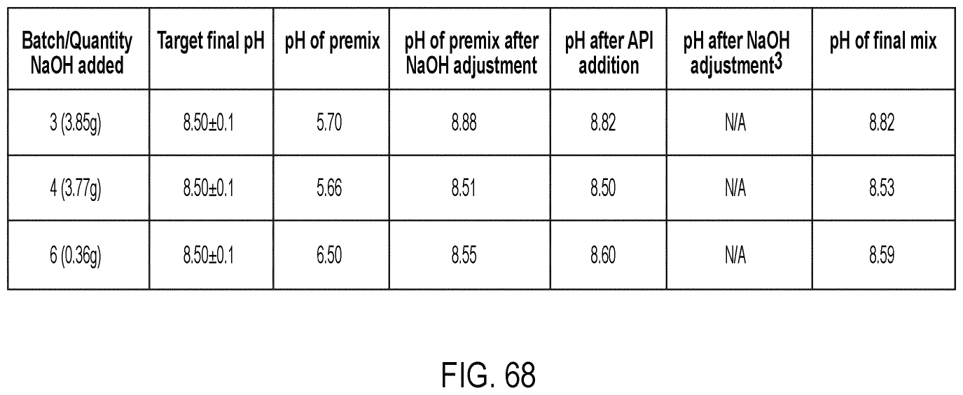

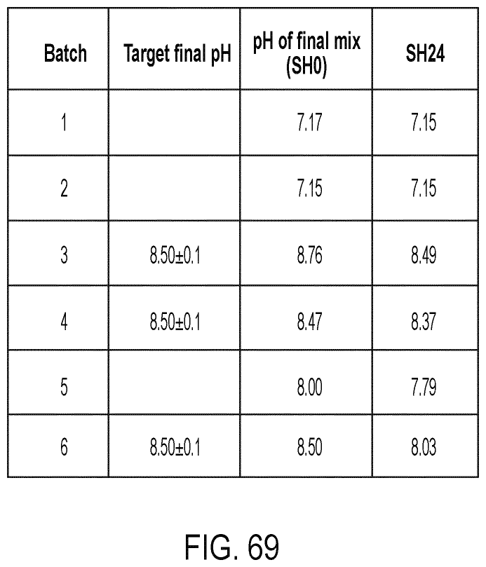

Sodium hydroxide (NaOH) 5% w/w solution was used to adjust the pH of the relevant mixes. The pH measurements that were taken for these batches at different stages of mix preparation are described in FIG. 68. The pH was successful adjusted to target pH for batches 4 and 6. The pH of batch 3 was adjusted above the target range in error; the impact of this deviation on chemical stability will be assessed when comparing data obtained from stability studies. The pH of each batch was also monitored at the specified suspension hold time points shown in FIG. 69. Results of pH monitoring over suspension holding periods show that the pH of the majority of mixes declined slightly with regards to their respective final mix (SH0) measurement. This decline is generally more marked in batches that were pH adjusted.

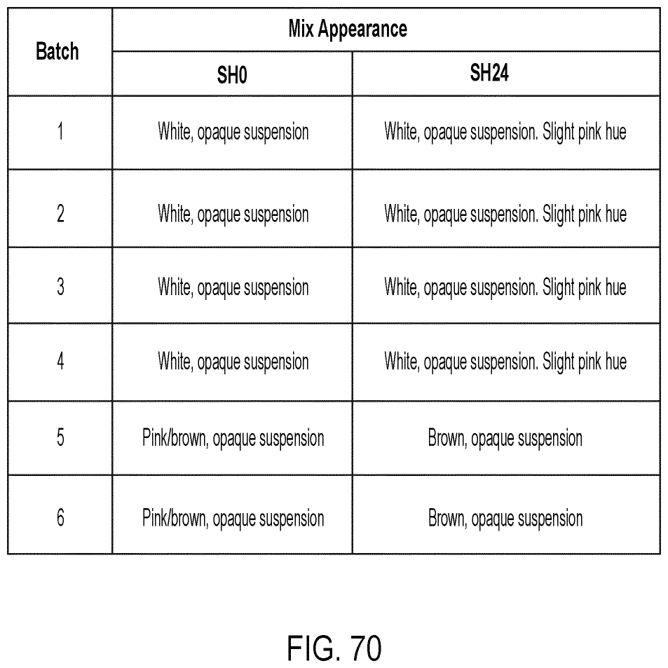

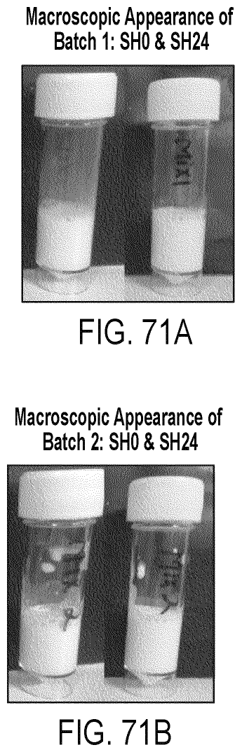

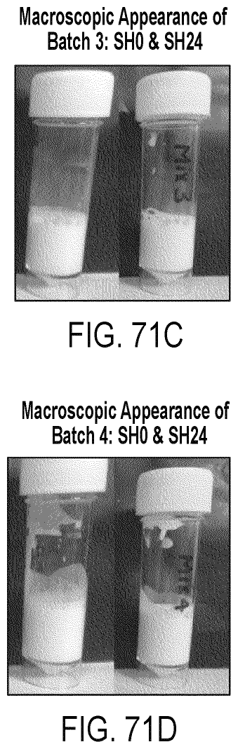

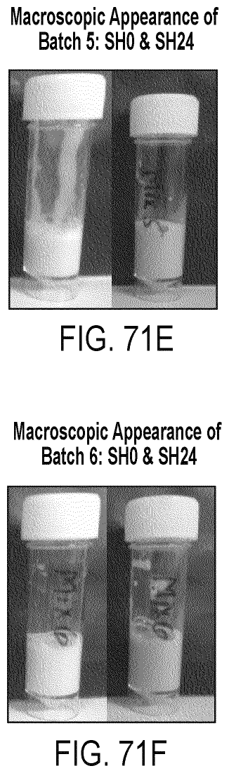

The appearances of the suspensions were recorded at the end of mixing (SH0) and after approximately 24 hours stirring (SH24). The observations recorded for each mix are summarized in FIG. 70 and illustrated in FIGS. 71A-F. Batches 1-4 produced white opaque suspensions which remained largely consistent in appearance over the hold period, although had developed a very slight pink hue at the SH24 time point. Batches 5 and 6 produced opaque suspensions with pink/brown discoloration present at the initial time point. A significant change was also evident after 24 hours of suspension hold; suspensions darkened further to a brown color. These batches were formulated without sodium metabisulfite and EDTA. The darker appearance of these samples is indicative of Epinephrine instability/oxidation, signifying that inclusion of antioxidant helps ensure chemical stability. The difference in pH levels between the formulations had no apparent effect on chemical stability/appearance with respect to macroscopic observations.

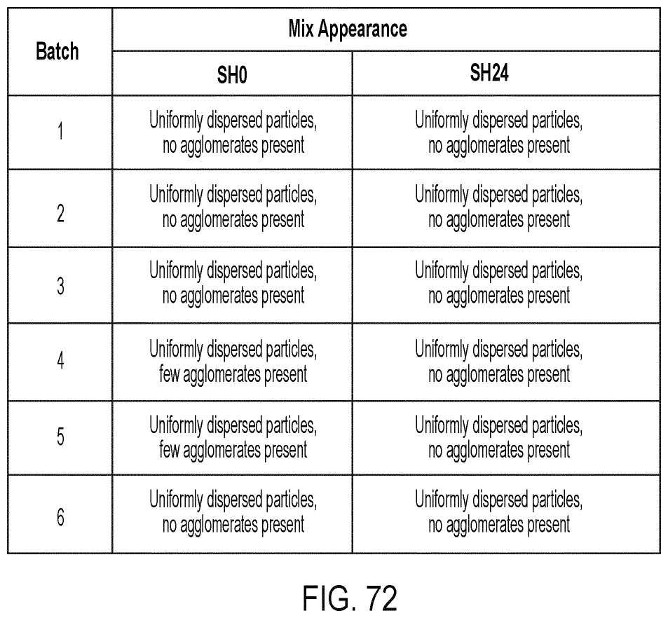

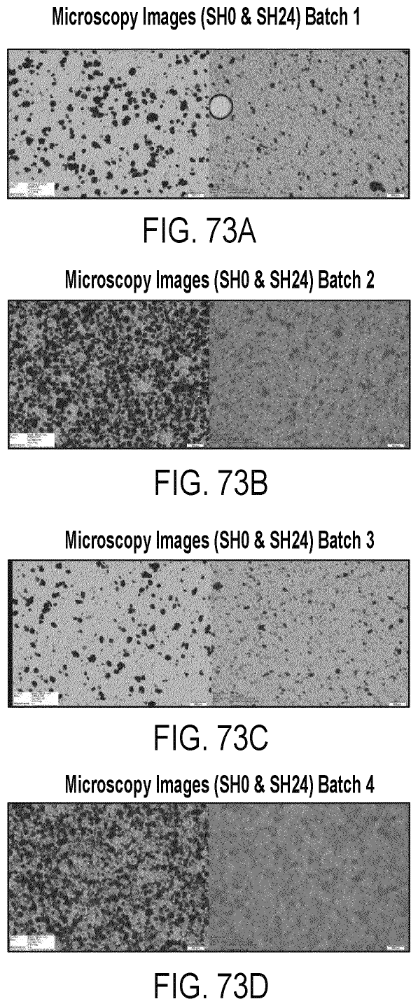

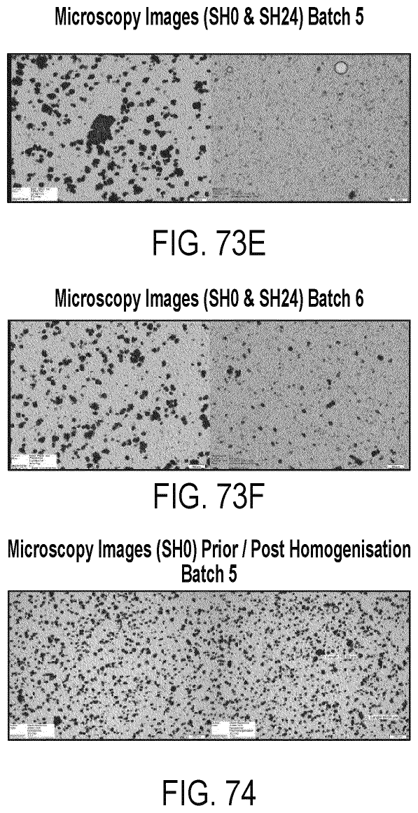

The microscopic appearance of the suspensions were examined at the end of mixing (SH0) and after approximately 24 hours hold time (SH24). The observations recorded for each mix are summarized in FIG. 72 and illustrated in FIGS. 73A-F (all images provided captured at ×10 magnification). Microscopy images show that each mix remains well dispersed over the holding period, although a small number of agglomerates/larger particles are present. The average particle size/degree of agglomeration appears to reduce after 24 hours of suspension holding. This may be a result of the continual shear caused by the motion of the magnetic stirrer, used to maintain mix homogeneity during the suspension hold period. Following the observation of agglomeration/large particles at the SH0 time point, batch 5 was homogenized at a speed of 4500 rpm for 1 minute in order to ascertain whether this alleviated agglomeration/improved the particle size distribution. Comparison of microscopy images prior and post homogenization are provided in FIG. 74. Both images were captured with ×5 magnification. Homogenization of the mix had no obvious benefit with regards to reducing agglomeration/particle size. Consequently, no further homogenization was performed on any of the batches.

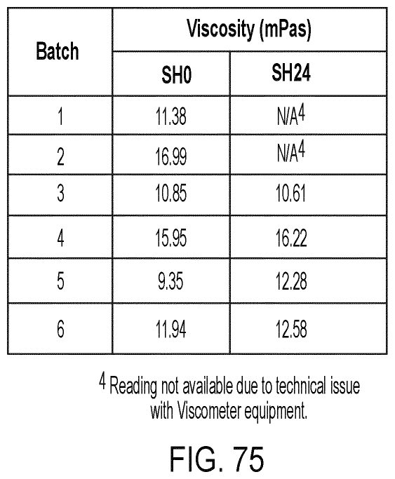

The viscosity of each mix was monitored across the suspension holding period using a Haake VT5500 viscometer. All tests were performed at 15° C., and readings provided at a shear rate of 500 s⁻¹. The results are provided in FIG. 75. The viscosity of the more concentrated batches (i.e., 13.33% w/w Epinephrine) is greater than that of less concentrated batches (i.e., 3.33% w/w) as is expected given the higher proportion of solids (powder) included in the formulation.

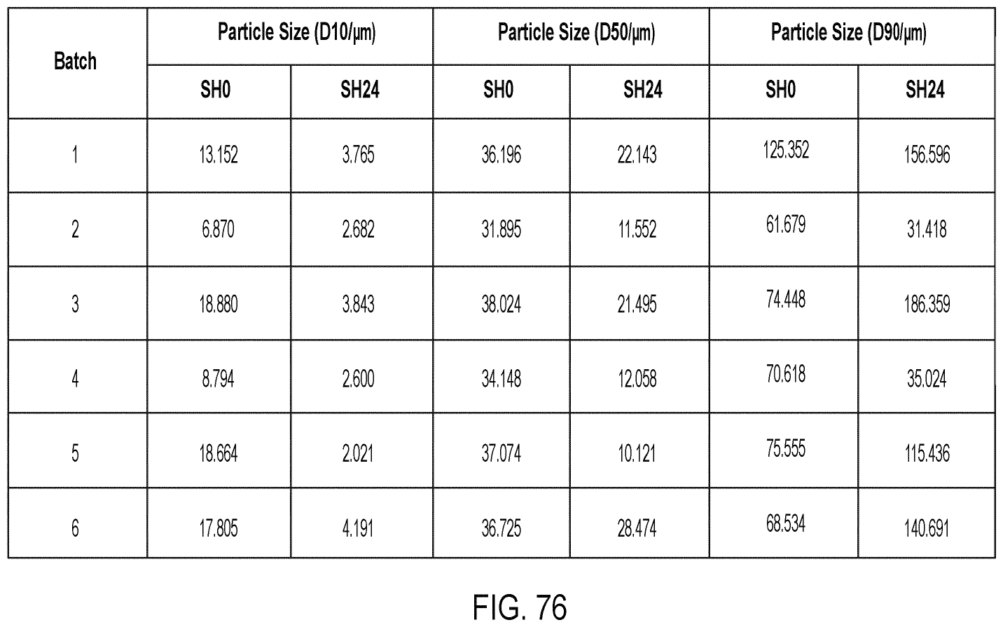

The particle size distribution of each mix was observed over the suspension hold period. Results are reported as D10, D50, and D90 values as shown in FIG. 76. The particle size analysis results suggest a general trend of particle size reduction over the holding period. This is suspected to be attributable to the continual shear caused by the motion of the magnetic stirrer. The variability of the results may also indicate a degree of settling within the suspension samples during analysis.

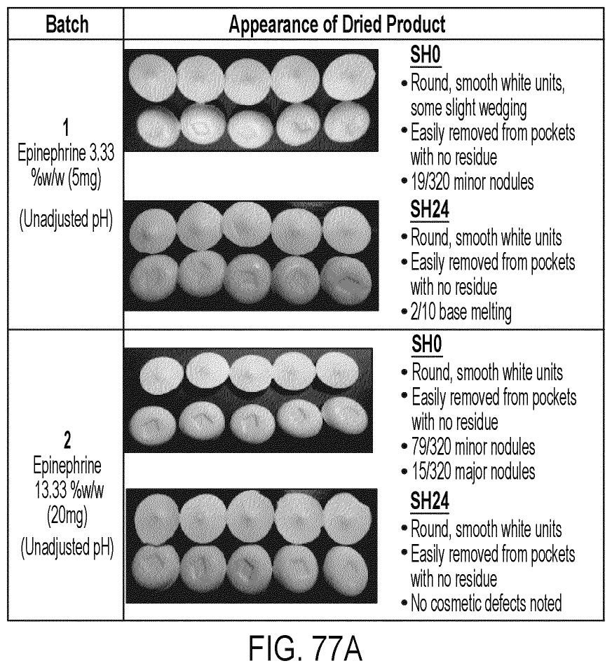

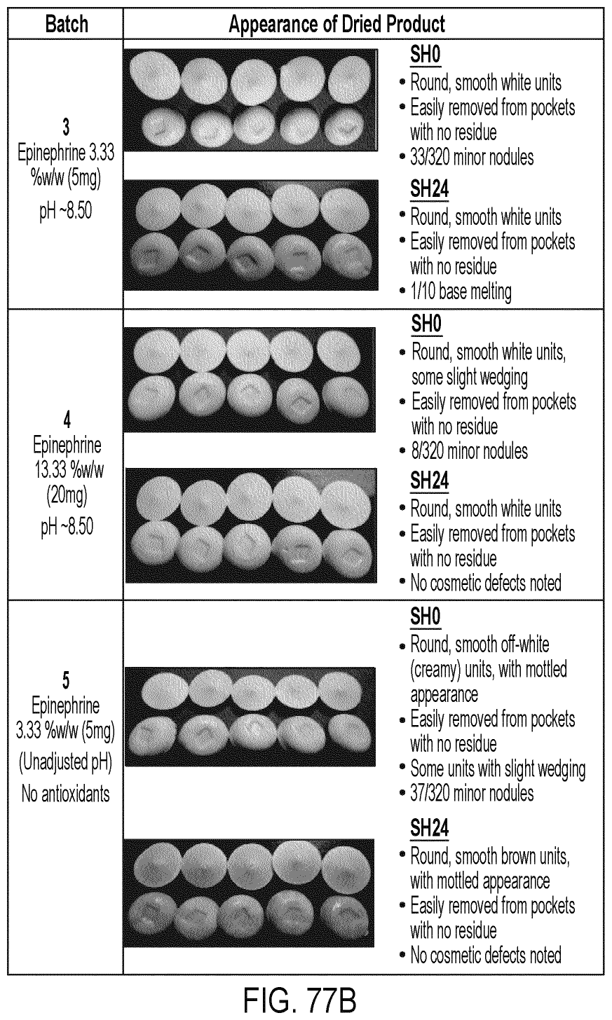

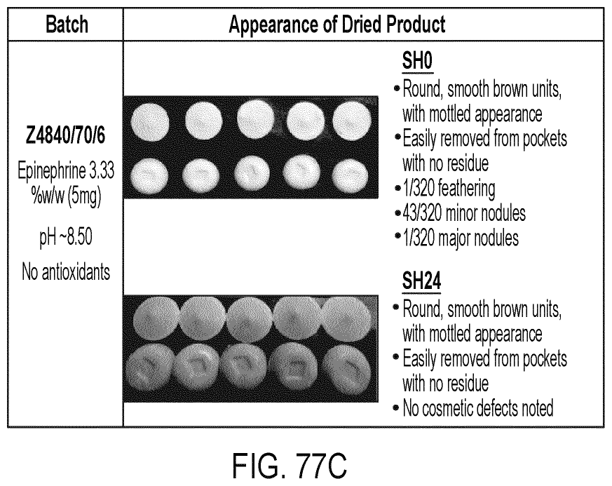

All batches were inspected and observations recorded in the batch manufacturing record. The appearance was considered acceptable if the units were circular, freeze-dried tablets that could be removed from packaging without breaking and were generally free of defects. The observations are shown in FIGS. 77A-C. Overall unit appearance for batches 1-4 was acceptable for the purposes of this study, with all batches producing smooth, round white units with some minor cosmetic defects noted. General unit appearance was notably improved compared to previous solution based formulation development studies. The appearance of batches 5-6 differed in that marked discoloration was present, resulting in production of yellow/brown units. This was expected given the macroscopic observations for these batches. The color change is indicative of chemical instability, thus signifying that inclusion of antioxidant in the formulation is required to prevent API degradation. A low occurrence of nodules was observed across number sub-batches; this is common observation when using fish gelatin and is also likely to be a result of bench-scale processing factors. A small amount of minor wedging was also observed for batches 1 and 4, which is again likely a result of bench-scaled manufacturing factors (i.e., hand dosing). Base melting was also noted during inspection of the units dosed at the SH24 time point from batches 1 and 3. This indicates that the drying cycle did not sufficiently dry all units.

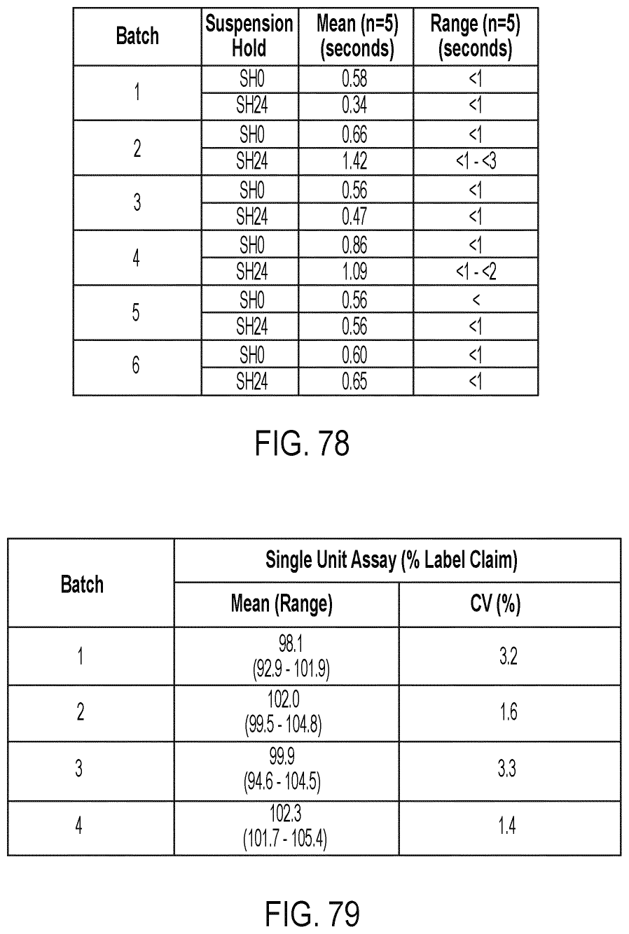

The dispersion time results for batches 1-6 are summarized in FIG. 78. The dispersion time is a measurement of the time required for a unit to fully wet. All batches dispersed within the acceptable limit of less than or equal to 10 seconds. The inclusion of sodium metabisulfite, EDTA, and difference in pH levels did not appear to affect the rate of dispersion for either dosage strength. The higher dosage strength formulations (2 and 4) have every slightly longer dispersion times, likely due to the high proportion of solids (powder).

During the initial timepoint (SH0) dosing operation of each batch, the dosing equipment was stopped for a period of 30 seconds and restarted without flushing the dosing lines. One tray (8 blisters) was then dosed. Ten units taken from locations across the tray were to be tested for single unit assay, allowing assessment of the rate/extent of suspension settling within the dosage lines and manifolds. Formulations 5 and 6 exhibited obvious chemical instability and will not be progressed to further development. Units from these batches were not tested. Results of the minimum stoppage/content uniformity study are shown in FIG. 79. The weight f each unit was also measured; units found to have outlying assay were noted to have variable tablet weights in parallel, thus can be considered a result of bench scale dosing processes (i.e., hand dosing). All single tablet assay results were within the range of 90 to 110% label claim. Results indicate that suspension settling is not a significant issue.

Suspension samples of batches 1-4 were collected following completion of dosing at the SH24 time point and analyzed to determine the effect of pH on the proportion of drug in suspension (solid state). Batches 5 and 6 were not analyzed as observations during manufacturing indicated obvious chemical instability. The drug is known to be more stable when in solid (crystalline) state and is less soluble at higher pH levels. Data obtained was used to determine if pH adjustment of the formulations is required to ensure optimal chemical stability. Samples were centrifuged, and the resultant supernatant analyzed for Epinephrine content thus determining the proportion of API present in solution. Results are summarized in Table 11 below:

TABLE 11

| | | | Supernatant | |
|---|---|---|---|---|
| | | | Epinephrine | % of Epinephrine |
| Sub- | Epinephrine | Final pH | Content | Dissolved in |
| Batch | (% w/w) | (SH24) | (% w/w) | Suspension Sample |
| 1 | 3.33 | 7.15 | 0.42 | 12.5 |
| 2 | 13.33 | 7.15 | 0.46 | 3.5 |
| 3 | 3.33 | 8.49 | 0.05 | 1.4 |
| 4 | 13.33 | 8.37 | 0.23 | 1.7 |

The pH unadjusted formulations (sub-batches 1-2) were found to have higher proportions of Epinephrine dissolved in the suspension samples compared to pH adjusted formulations. The supernatant Epinephrine content of each batch is close in value despite the difference in formulation Epinephrine content; this is thought to be because the solubility of Epinephrine in a pH adjusted formulation will be largely unchanged, with larger quantities of API added not affecting this as the solubility limit has been reached. Results indicate that the pH adjusted formulation is likely to be more stable due to the higher proportion of the API in suspension (i.e. solid/crystalline form).

Sealed blisters of units dosed after 24 hours of suspension hold (SH24) were analyzed to provide information on the in-process stability during the holding period. Analytical evaluation included Epinephrine assay, Sodium metabisulfite assay and related substances testing. Study results from testing of the SH24 samples can be compared to those from testing of respective SH0 samples to assess in-process stability of each formulation. These studies were performed on sub-batches 1-4. Sub-batches 5-6 were excluded due to obvious chemical instability. The following table 12 provides the in-process stability data for sub-batches 1-4:

TABLE 12

| | | Epinephrine | Sodium | Impurity F | Total Impurities |
|---|---|---|---|---|---|
| | | (% Label | metabisulfite | (RRT~0.78) | (Unknown + |
| Batch | | Claim) | (% Label Claim) | (%) | Impurity F) (%) |
| Sub-batch 1 | SH0 | 101.1 | 68.2 | 2.09 | 2.20 |
| (3.33% w/w | SH24 | 98.0 | 51.1 | 1.93 | 2.05 |
| Epinephrine, | | | | | |
| pH unadjusted) | | | | | |
| Sub-batch 2 | SH0 | 102.5 | 61.6 | 0.44 | 0.44 |
| (13.33% w/w | SH24 | 99.2 | 32.3 | 0.46 | 0.56 |
| Epinephrine, | | | | | |
| pH unadjusted) | | | | | |
| Sub-batch 3 | SH0 | 101.2 | 85.3 | Not quantifiable | Not quantifiable |
| (3.33% w/w | | | | (<0.10%) | (<0.10%) |
| Epinephrine, | SH24 | 98.6 | 82.0 | 0.13 | 0.13 |
| pH ~8.5) | | | | | |
| Sub-batch 4 | SH0 | 103.5 | 84.3 | None detected | None detected |
| (13.33% w/w | | | | (<0.05%) | (<0.05%) |
| Epinephrine, | SH24 | 101.0 | 60.9 | Not quantifiable | Not quantifiable |
| pH ~8.5) | | | | (<0.10%) | (<0.10%) |

In-process stability results show improvement in comparison to previous studies of solution-based formulations above. For example, the rate of Sodium metabisulfite consumption is lower in both the pH unadjusted and pH adjusted batches relative to the difference in tablet loading between suspension and solution-based formulations (0.45 mg and up to 1.05 mg respectively, dependent on fill volume). A lower degree of impurity F and unknown impurity formation is also evident. This is particularly pronounced in the pH adjusted formulations, indicating that the higher pH provides optimal stability (as with the results of the suspension/solution analyses).

Following completion of manufacture, sealed blisters of sub-batches 1-6 (dosed at 0 hours suspension hold, SH0) underwent stability studies. Blisters were to be stored at both 25° C./60% RH and 40° C./75% RH for up to three months to evaluate chemical stability characteristics and determine whether previous stability concerns were alleviated by the change in formulation strategy. Samples were to be pulled and analyzed at monthly time points. The results of these studies are to be used to identify the optimal formulation with regards to stability for progression to clinical supply manufacture, and to determine the requirements for future development studies. Assay results from 5 mg, unadjusted sub-batch 1 samples at each timepoint and for each respective condition are provided in the following tables 13-14:

TABLE 13
(25° C./60% RH)

| | Epinephrine | Sodium | Impurity F | Total Impurities |
|---|---|---|---|---|
| | (% Label | metabisulfite | (RRT~0.78) | (Unknown + |
| Time Point | Claim) | (% Label Claim) | (%) | Impurity F) (%) |
| Initial | 101.1 | 68.2 | 2.09 | 2.20 |
| (Samples stored for | | | | |
| 1 month at 5° C. | | | | |
| used as pseudo | | | | |
| T = 0 timepoint) | | | | |
| Month 1 | 100.1 | 58.4 | 4.37 | 4.47 |
| Month 2 | 99.0 | 51.9 | 6.57 | 6.67 |
| Month 3 | 97.1 | 42.7 | 6.94 | 7.07 |

TABLE 14
(40° C./75% RH)

| | Epinephrine | Sodium | Impurity F | Total Impurities |
|---|---|---|---|---|
| | (% Label | metabisulfite | (RRT~0.78) | (Unknown + |
| Time Point | Claim) | (% Label Claim) | (%) | Impurity F) (%) |
| Initial | 101.1 | 68.2 | 2.09 | 2.20 |
| (Samples stored for | | | | |
| 1 month at 5° C. | | | | |
| used as pseudo | | | | |
| T = 0 timepoint) | | | | |
| Month 1 | 92.7 | 20.9 | 13.38 | 13.61 |
| Month 2 | 93.7 | 10.9 | 13.49 | 14.02 |
| Month 3 | 92.7 | 11.7 | 12.16 | 12.71 |

Analytical data shows that the assay recovery remains within acceptable limits throughout the stability storage periods at both conditions, although displays more significant loss at 40° C./75% RH (˜8.4%). Initial Sodium metabisulfite assay values display a degree of consumption during manufacture, and show that consumption continues during the storage period (more pronounced at 40° C./75% RH). In conjunction with the decline in Sodium metabisulfite assay values, the levels of Impurity F and other unknown impurities also increase. This formulation does not show a marked improvement in stability in comparison to previous studies of the same nature involving solution-based formulations. These results indicate that finished product from this formulation is not well protected against oxidation and may not have a suitable shelf life.

Assay results from 20 mg, unadjusted pH sub-batch 2 samples at each timepoint and for each respective condition are provided in the following tables 15-16:

TABLE 15
(25° C./60% RH)

| | Epinephrine | Sodium | Impurity F | Total Impurities |
|---|---|---|---|---|
| | (% Label | metabisulfite | (RRT~0.78) | (Unknown + |
| Time Point | Claim) | (% Label Claim) | (%) | Impurity F) (%) |
| Initial | 102.5 | 61.6 | 0.44 | 0.44 |
| (Samples stored for | | | | |
| 1 month at 5° C. | | | | |
| used as pseudo | | | | |
| T = 0 timepoint) | | | | |
| Month 1 | 101.8 | 54.2 | 1.15 | 1.15 |
| Month 2 | 101.3 | 34.1 | 1.55 | 1.55 |
| Month 3 | 103.1 | 31.2 | 1.69 | 1.69 |

TABLE 16
(40° C./75% RH)

| | Epinephrine | Sodium | Impurity F | Total Impurities |
|---|---|---|---|---|
| | (% Label | metabisulfite | (RRT~0.78) | (Unknown + |
| Time Point | Claim) | (% Label Claim) | (%) | Impurity F) (%) |
| Initial | 102.5 | 61.6 | 0.44 | 0.44 |
| (Samples stored for | | | | |
| 1 month at 5° C. | | | | |
| used as pseudo | | | | |
| T = 0 timepoint) | | | | |
| Month 1 | 101.0 | 19.5 | 2.99 | 2.99 |
| Month 2 | 97.3 | 3.0 | 3.24 | 3.24 |
| Month 3 | 102.3 | 5.1 | 3.22 | 3.22 |

Analytical data shows that the assay recovery remains within acceptable limits throughout the stability storage period at both conditions. Initial Sodium metabisulfite assay values display a degree of consumption during manufacture, and show that consumption continues during the storage period (more pronounced at 40° C./75% RH). In conjunction with the decline in Sodium metabisulfite assay values, the levels of Impurity F increase. The absence of both assay loss and accumulation of unknown impurities suggests that this formulation is superior to the equivalent lower dosage strength formulation (5 mg). This formulation shows improved stability with respect to sub-batch 1 (expected given relative proportion of API in solution and higher unit solid content, 5 mg vs. 20 mg). There is also a marked improvement in stability in comparison to previous studies of the same nature involving solution-based formulations. This suggests that the improvement in stability is inherent in the change in formulation strategy (API more stable in the solid state).

Assay results from 5 mg, pH 8.5 sub-batch 3 samples at each timepoint and for each respective condition are provided in the following tables 17-18:

TABLE 17
(25° C./60% RH)

| | Epinephrine | Sodium | Impurity F | Total Impurities |
|---|---|---|---|---|
| | (% Label | metabisulfite | (RRT~0.78) | (Unknown + |
| Time Point | Claim) | (% Label Claim) | (%) | Impurity F) (%) |
| Initial | 101.2 | 85.3 | Not quantifiable | Not quantifiable |
| (Samples stored for | | | (<0.10%) | (<0.10%) |
| 1 month at 5° C. | | | | |
| used as pseudo | | | | |
| T = 0 timepoint) | | | | |
| Month 1 | 101.5 | 83.3 | 0.19 | 0.19 |
| Month 2 | 100.6 | 82.4 | 0.25 | 0.38 |
| Month 3 | 100.0 | 81.8 | 0.23 | 0.50 |

TABLE 18
(40° C./75% RH)

| | Epinephrine | Sodium | Impurity F | Total Impurities |
|---|---|---|---|---|
| | (% Label | metabisulfite | (RRT~0.78) | (Unknown + |
| Time Point | Claim) | (% Label Claim) | (%) | Impurity F) (%) |
| Initial | 101.2 | 85.3 | Not quantifiable | Not quantifiable |
| (Samples stored for | | | (<0.10%) | (<0.10%) |
| 1 month at 5° C. | | | | |
| used as pseudo | | | | |
| T = 0 timepoint) | | | | |
| Month 1 | 100.8 | 79.6 | 0.54 | 0.54 |
| Month 2 | 96.1 | 77.8 | 0.56 | 0.66 |
| Month 3 | 100.8 | 75.9 | 0.59 | 0.85 |

Analytical data shows that the assay recovery remains within acceptable limits throughout the stability storage period at both conditions. Initial Sodium metabisulfite assay values indicate a lower rate of consumption both during manufacture and during the storage period. Whilst the levels of Impurity F and unknown impurities increase in conjunction with the decline in Sodium metabisulfite assay values, this is lower than in pH unadjusted formulations (sub-batches 1-2) and remains below limits specified by the BP monograph for the Epinephrine injection (reference product). This formulation shows marked improvement in stability in comparison to the pH unadjusted formulation, and previous studies of involving solution-based formulations. These results indicate that pH adjustment is required to provide optimal stability characteristics, as also indicated by solution/suspension analyses and evaluation of in-process data.

Assay results from 20 mg, pH 8.5 sub-batch 4 samples at each timepoint and for each respective condition are provided in the following tables 19-20:

TABLE 19
(25° C./60% RH)

| | Epinephrine | Sodium | Impurity F | Total Impurities |
|---|---|---|---|---|
| | (% Label | metabisulfite | (RRT~0.78) | (Unknown + |
| Time Point | Claim) | (% Label Claim) | (%) | Impurity F) (%) |
| Initial | 103.5 | 84.3 | None detected | None detected |
| (Samples stored for | | | (<0.05%) | (<0.05%) |
| 1 month at 5° C. | | | | |
| used as pseudo | | | | |
| T = 0 timepoint) | | | | |
| Month 1 | 102.8 | 82.6 | Not quantifiable | Not quantifiable |
| | | | (<0.10%) | (<0.10%) |
| Month 2 | 102.4 | 71.6 | 0.10 | 0.10 |
| Month 3 | 106.2 | 73.9 | 0.10 | 0.10 |

TABLE 20
(40° C./75% RH)

| | Epinephrine | Sodium | Impurity F | Total Impurities |
|---|---|---|---|---|
| | (% Label | metabisulfite | (RRT~0.78) | (Unknown + |
| Time Point | Claim) | (% Label Claim) | (%) | Impurity F) (%) |
| Initial | 103.5 | 84.3 | None detected | None detected |
| (Samples stored for | | | (<0.05%) | (<0.05%) |
| 1 month at 5° C. | | | | |
| used as pseudo | | | | |
| T = 0 timepoint) | | | | |
| Month 1 | 103.6 | 77.9 | 0.20 | 0.20 |
| Month 2 | 102.8 | 63.8 | 0.24 | 0.24 |
| Month 3 | 105.4 | 63.4 | 0.26 | 0.26 |

Analytical data shows that the assay recovery remains within acceptable limits throughout the stability storage period at both conditions. Initial Sodium metabisulfite assay values indicate a lower rate of consumption both during manufacture and during the storage period compared to pH unadjusted formulations. The Sodium metabisulfite is consumed at a higher rate than seen in the equivalent formulation with a lower dosage strength; this is expected given the higher drug loading in this formulation. Whilst the levels of Impurity F and unknown impurities increase in conjunction with the decline in Sodium metabisulfite assay values, this is lower than in pH unadjusted formulations (sub-batches 1-2) and remains below limits specified by the BP monograph for the Epinephrine injection (reference product). This formulation shows marked improvement in stability in comparison to the pH unadjusted formulation, and previous studies of involving solution-based formulations. These results indicate that pH adjustment is required to provide optimal stability characteristics, as also indicated by solution/suspension analyses and evaluation of in-process data.

Assay results from 5 mg, unadjusted pH, no antioxidants sub-batch 5 samples at each timepoint and for each respective condition are provided in the following tables 21-22:

TABLE 21
(25° C./60% RH)

| | Epinephrine | Total Unknown Impurities |
|---|---|---|
| Time Point | (% Label Claim) | (%) |
| Initial | 101.2 | None detected (<0.05%) |
| (Samples stored for | | |
| 1 month at 5° C. | | |
| used as pseudo | | |
| T = 0 timepoint) | | |
| Month 1 | 101.5 | None detected (<0.05%) |
| Month 2 | 102.1 | 0.13 |
| Month 3 | 101.9 | None detected (<0.05%) |

TABLE 22
(40° C./75% RH)

| | Epinephrine | Total Unknown Impurities |
|---|---|---|
| Time Point | (% Label Claim) | (%) |
| Initial | 101.2 | None detected (<0.05%) |
| (Samples stored for | | |
| 1 month at 5° C. | | |
| used as pseudo | | |
| T = 0 timepoint) | | |
| Month 1 | 102.1 | Not quantifiable (<0.10%) |
| Month 2 | 100.9 | Not quantifiable (<0.10%) |
| Month 3 | 102.6 | None detected (<0.05%) |

Analytical data shows that the assay recovery remains within acceptable limits throughout the stability storage period at both conditions. The levels of unknown impurities also do not show any consistent increase during the storage period (Impurity F not present as formulated without Sodium metabisulfite). This formulation shows marked improvement in stability in comparison to previous studies of involving solution-based formulations. This suggests that the improvement in stability is inherent in the change in formulation strategy (API more stable in the solid state). Although data obtained during the storage period suggests the formulation has acceptable stability, it should be noted that all samples placed on storage were dosed at the 0-hour suspension hold timepoint (SH0), thus not providing an indication of the in-process stability. During manufacture this formulation was seen to display significant discoloration, indicating insufficient stability and supporting the requirement to include preservatives (Sodium metabisulfite and EDTA).

Assay results from 5 mg, pH 8.5, no antioxidants sub-batch 6 samples at each timepoint and for each respective condition are provided in the following tables 23-24:

TABLE 23
(25° C./60% RH)

| | Epinephrine | Total Unknown Impurities |
|---|---|---|
| Time Point | (% Label Claim) | (%) |
| Initial | 102.1 | None detected (<0.05%) |
| (Samples stored for | | |
| 1 month at 5° C. | | |
| used as pseudo | | |
| T = 0 timepoint) | | |
| Month 1 | 101.7 | None detected (<0.05%) |
| Month 2 | 101.7 | 0.43 |
| Month 3 | 101.9 | None detected (<0.05%) |

TABLE 24
(40° C./75% RH)

| | Epinephrine | Total Unknown Impurities |
|---|---|---|
| Time Point | (% Label Claim) | (%) |
| Initial | 102.1 | None detected (<0.05%) |
| (Samples stored for | | |
| 1 month at 5° C. | | |
| used as pseudo | | |
| T = 0 timepoint) | | |
| Month 1 | 102.4 | None detected (<0.05%) |
| Month 2 | 101.2 | Not quantifiable (<0.10%) |
| Month 3 | 103.2 | None detected (<0.05%) |

Analytical data shows that the assay recovery remains within acceptable limits throughout the stability storage period at both conditions. The levels of unknown impurities also do not show any consistent increase during the storage period (Impurity F not present as formulated without Sodium metabisulfite). This formulation shows marked improvement in stability in comparison to previous studies of involving solution-based formulations. This suggests that the improvement in stability is inherent in the change in formulation strategy (API more stable in the solid state). Although data obtained during the storage period suggests the formulation has acceptable stability, it should be noted that all samples placed on storage were dosed at the 0-hour suspension hold timepoint (SH0), thus not providing an indication of the in-process stability. During manufacture this formulation was seen to display significant discoloration, indicating insufficient stability and supporting the requirement to include preservatives (Sodium metabisulfite and EDTA).

### Suspension-Based Formulation Study 2

The objective of this study was to assess the manufacturability of the solution-based Epinephrine formulation at a similar scale to that of a bench scale clinical manufacture. The formulation to be manufactured was selected based on results and observations from the previous formulation development study. The higher dosage strength (13.33% w/w; 20 mg/unit) was selected to represent worst-case conditions with regards to the capability of the manufacturing process and equipment, as well as microbiological risk. Samples of the finished product under went chemical stability testing. Critical physical attributes of the dried units were also to be evaluated, including appearance and dispersion time.

During the previous reformulation study, a suitably stable Epinephrine suspension formulation was identified (applicable to both 5 mg and 20 mg dosage strengths). This formulation was selected to progress to preclinical (technical) batch manufacture in order to establish the feasibility of manufacturing at a larger scale, using similar processing conditions as would be employed during bench scale clinical manufacturing. The outcomes of this study were to inform the process conditions and set points to be taken forward to future campaigns.

To allow assessment of the manufacturability of the proposed Epinephrine formulations, one technical batch was to be produced (higher dosage strength, representing worst case with regards to process) with the aim of:
Evaluating the feasibility of manufacturing the proposed Zydis® Epinephrine formulation at a scale similar to that of a bench scale clinical manufacture;
Establishing a suitable drying cycle for the Zydis® Epinephrine formulation following observations of base melting during previous reformulation study; and
Providing samples to the analytical department for evaluation of finished product chemical stability.

The batches was to be dosed at the end of mixing (0 hours suspension hold, SH0). The product had a minimum of 6 hours frozen hold (FH) prior to freeze drying. The following Table 25 includes the formulation used for this study:

TABLE 25

| Batch | Unit Wet | Gelatin | | Sodium | pH | |
|---|---|---|---|---|---|---|
| Size | Fill Weight | (Fish HMW) | EDTA | Metabisulfite | (NaOH for | Epinephrine |
| (g) | (mg) | (% w/w) | (% w/w) | (% w/w) | adjustment) | (% w/w) |
| 800 | 150 | 4.30 | 0.05 | 0.30 | 8.50 ± 0.2 | 13.33 |

Assay results for this study can be found in the following Table 26:

TABLE 26

| Test | Specification | Result |
|---|---|---|
| Sodium | For information only (% label claim) | 98.8% |
| Metabisulfite Assay | | |
| Epinephrine Assay | For information only (% label claim) | 101.8% |
| Related Substances | Impurity F (Epinephrine Sulfonate): | 0.1% |
| | For information only (%) | |
| | Impurity B (Norepinephrine): For | None |
| | information only (%) | detected |
| | Unknown individual impurity: For | 0.1% (RRT |
| | information only (%) | 0.10) |
| | Total of all individual impurities | 0.1% |
| | (exclusive of impurity F): For | |
| | information only (%) | |

Analytical testing results for assay and related substances have shown the finished product in this study is of acceptable quality.

### Additional Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Reference to “about” a value or parameter herein includes (and describes) variations that are directed to that value or parameter per se. For example, description referring to “about X” includes description of “X”. In addition, reference to phrases “less than”, “greater than”, “at most”, “at least”, “less than or equal to”, “greater than or equal to”, or other similar phrases followed by a string of values or parameters is meant to apply the phrase to each value or parameter in the string of values or parameters. For example, a statement that a solution has a concentration of at least about 10 mM, about 15 mM, or about 20 mM is meant to mean that the solution has a concentration of at least about 10 mM, at least about 15 mM, or at least about 20 mM.

As used herein, the singular forms “a,” “an,” and “the” are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

This application discloses several numerical ranges in the text and figures. The numerical ranges disclosed inherently support any range or value within the disclosed numerical ranges, including the endpoints, even though a precise range limitation is not stated verbatim in the specification because this disclosure can be practiced throughout the disclosed numerical ranges.

The above description is presented to enable a person skilled in the art to make and use the disclosure, and is provided in the context of a particular application and its requirements. Various modifications to the preferred embodiments will be readily apparent to those skilled in the art, and the generic principles defined herein may be applied to other embodiments and applications without departing from the spirit and scope of the disclosure. Thus, this disclosure is not intended to be limited to the embodiments shown, but is to be accorded the widest scope consistent with the principles and features disclosed herein.

1. An oral solid dosage form comprising:
an Epinephrine hormone or pharmaceutically acceptable salt or solvate thereof;
10-40 wt. % of a matrix former;
10-40 wt. % of a structure former; and
an antioxidant.

2. The dosage form of claim 1, wherein the antioxidant comprises sodium metabisulfite.

3. The dosage form of claim 1, wherein the dosage form comprises 0.1-5 wt. % of the antioxidant.

4. The dosage form of claim 1, further comprising a chelating agent.

5. The dosage form of claim 4, wherein the chelating agent comprises edetate disodium (“EDTA”).

6. The dosage form of claim 4, wherein the dosage form comprises 0.1-1 wt. % the chelating agent.

7. The dosage form of claim 1, wherein the matrix former comprises gelatin, pullulan, starch, or combinations thereof.

8. The dosage form of claim 7, wherein the gelatin comprises fish gelatin, bovine gelatin, porcine gelatin, or combination thereof.

9. The dosage form of claim 8, wherein the gelatin is fish gelatin and the fish gelatin is high molecular weight fish gelatin.

10. The dosage form of claim 1, wherein the structure former comprises mannitol.

11. The dosage form of claim 1, wherein the Epinephrine hormone or pharmaceutically acceptable salt or solvate thereof comprises Epinephrine maleate, Epinephrine tartrate, Epinephrine Bitartrate, or Epinephrine hydrochloride.

12. The dosage form of claim 1, wherein the dosage form comprises a pharmaceutically effective amount of the Epinephrine hormone or pharmaceutically acceptable salt or solvate thereof.

13. The dosage form of claim 1, wherein the dosage form comprises 15-70 wt. % of the Epinephrine hormone or pharmaceutically acceptable salt or solvate thereof.

14. The dosage form of claim 1, further comprising a pH modifier, wherein the pH modifier is sodium hydroxide.

15. The dosage form of claim 13, wherein the dosage form comprises 0.1-2 wt. % pH modifier.

16. The dosage form of claim 1, further comprising a sweetener.

17. The dosage form of claim 16, wherein the sweetener is sucralose.

18. The dosage form of claim 16, wherein the dosage form comprises 0.1-5 wt. % sweetener

19. A method of treating a patient, the method comprising placing the dosage form of claim 1 in an oral cavity of a person in need of the treatment.

20. The method of claim 19, wherein placement in the oral cavity is placement on or under the tongue or in the buccal or pharyngeal region.

21. A method of forming an oral solid dosage form, the method comprises:
dosing a pharmaceutical formulation into a preformed mold, wherein the pharmaceutical formulation has a pH of 7.5-9.5 and the pharmaceutical formulation comprises:
an Epinephrine hormone or pharmaceutically acceptable salt or solvate thereof;
1-10 wt. % matrix former;
1-10 wt. % of a structure former;
0.1-1 wt. % antioxidant; and
a pH modifier;
freezing the dosed pharmaceutical formulation; and
freeze-drying the annealed pharmaceutical formulation to form the dosage form.

22. The method of claim 21, wherein the dosed pharmaceutical formulation is frozen at a temperature of −40° C. to −120° C. for a duration of about 1-5 minutes.

23. The method of claim 21, wherein the pharmaceutical formulation comprises a 0.01-0.1 wt. % chelating agent.

24-36. (canceled)

37. A method of forming a pharmaceutical formulation comprising:
mixing a solvent, a matrix former, and a structure former to form a pre-mix;
adding an antioxidant to the pre-mix;
adding a first pH modifier to the pre-mix such that the pH of the pre-mix is 7.15-9.5,
wetting an Epinephrine hormone or pharmaceutically acceptable salt or solvate thereof with the pre-mix to form a suspension; and
adding a second pH modifier to the suspension to form the pharmaceutical formulation such that the pH of the pharmaceutical formulation is 7.15-9.5, wherein the amount of the Epinephrine hormone or pharmaceutically acceptable salt or solvate thereof dissolved in the pharmaceutical formulation is less than about 3.5 wt. % as a proportion of epinephrine content.

38. The method of claim 37, wherein a chelating agent is also added to the pre-mix with the antioxidant.

39. The method of claim 37, further comprising heating the pre-mix to 50-70° C. and cooling the pre-mix to 15-30° C. after mixing the solvent, the matrix former, and the structure former.

40-41. (canceled)
